(19) United States
(12) Patent Application Publication
(10) Pub. No.: US 2026/0108547 A1
(43) Pub. Date: Apr. 23, 2026

(54) LEVERAGING L-FUCOSE-MEDIATED SIGNALING TO INDUCE MONOCYTE-DERIVED DENDRITIC CELL POLARIZATION

(71) Applicant: H. LEE MOFFITT CANCER CENTER AND RESEARCH INSTITUTE, INC., Tampa, FL (US)

(72) Inventor: Eric LE-LAU, Tampa, FL (US)

(21) Appl. No.: 19/118,148

(22) PCT Filed: Oct. 4, 2023
(86) PCT No.: PCT/US2023/076014

§ 371 Date: Apr. 3, 2025

Related U.S. Application Data
(60) Provisional application No. 63/413,002, filed on Oct. 4, 2022.
(60) Provisional application No. 63/416,279, filed on Oct. 14, 2022.

(51) Int. Cl. A61K 31/7004; A61K 39/00; A61K 39/395; A61K 40/42; A61P 35/00

(52) U.S. Cl. CPC A61K 31/7004; A61K 39/3955; A61K 40/42; A61P 35/00; A61K 2039/505

(57) ABSTRACT

Disclosed are methods for treating infectious diseases and cancers comprising administering to a subject a L-fucose.

### CROSS REFERENCE TO RELATED APPLICATIONS

This application claims the benefit of U.S. Provisional Application No. 63/416,279, filed on Oct. 14, 2023 and U.S. Provisional Application No. 63/413,002, filed on Oct. 4, 2023, application which are incorporated herein by in their entireties.

### STATEMENT OF GOVERNMENT SUPPORT

This invention was made with government support under Grant No. R01CA241559 awarded by NIH. The government has certain rights in the invention.

### REFERENCE TO SEQUENCE LISTING

The sequence listing submitted on Oct. 4, 2023, as an .XML file entitled “10110_428WO1.xml” created on Oct. 4, 2023, and having a file size of 24,576 bytes is hereby incorporated by reference pursuant to 37 C.F.R. § 1.52(e)(5).

### BACKGROUND

Dendritic cells (DCs) are a key contributor to anti-tumor immunity and can function as an intermediary between the innate and adaptive immune responses. The presence of DCs in the tumor microenvironment (TME) is generally associated with favorable prognosis across multiple cancer types. However, as tumors develop, various factors in the TME can suppress numbers and functions of DCs in mediating anti-tumor immune responses. For this reason, there is a need for new therapeutic modalities that can safely enhance the abundance and/or functionality of intratumoral DCs.

### SUMMARY

Disclosed are methods and compositions related to enhancing immune responses and treating cancers and infectious diseases with the administration of fucose.

In one aspect, disclosed herein are methods of increasing the number of monocyte-derived dendritic cells (moDCs) in a tumor microenvironment (such as, for example, tumor microenvironment of a melanoma or breast cancer) or site of infection of an infectious disease in a subject comprising administering to the subject an agent that increases the amount of fucosylation on myeloid cells (such as, for example, a fucose including, but not limited to L-fucose, D-fucose, fucose-1-phosphate, or GDP-L-fucose). In one aspect the fucose is not the fucosylation inhibitor 2-fluoro-fucose (2FF). In some aspects, the method can further comprise administering to the subject an autologous dendritic cell.

Also disclosed herein are methods of increasing the number of monocyte-derived dendritic cells (moDCs) of any preceding aspect, further comprising administering to the subject an immune checkpoint blockade inhibitor (such as, for example, PD-1 inhibitors lambrolizumab, OPDIVO® (Nivolumab), KEYTRUDA® (pembrolizumab), and/or pidilizumab; the PD-L1 inhibitors BMS-936559, TECENTRIQ® (Atezolizumab), IMFINZI® (Durvalumab), and/or BAVENCIO® (Avelumab); and/or the CTLA-4 inhibitor YERVOY (ipilimumab)). In one aspect, the fucose increasing agent is administered before and/or contiguous with administration of the immune checkpoint inhibitor.

In one aspect, disclosed herein are methods of increasing the number of monocyte-derived dendritic cells (moDCs) of any preceding aspect, further comprising administering to the subject an adoptive cell therapy (such as, for example the transfer of tumor infiltrating lymphocytes (TILs), tumor infiltrating NK cells (TINKs), dendritic cell (DC), marrow infiltrating lymphocytes (MILs), chimeric antigen receptor (CAR) T cells, and/or CAR NK cells).

Also disclosed herein are methods of increasing the number of monocyte-derived dendritic cells (moDCs) of any preceding aspect, wherein the infectious disease comprises an infection from a virus selected from the group of viruses consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In one aspect, disclosed herein are methods of increasing the number of monocyte-derived dendritic cells (moDCs) of any preceding aspect, wherein the infectious disease comprises an infection from a bacteria selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

Also disclosed herein are methods of increasing the number of monocyte-derived dendritic cells (moDCs) of any preceding aspect, wherein the infectious disease comprises an infection from a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

In one aspect, disclosed herein are methods of increasing the number of monocyte-derived dendritic cells (moDCs) of any preceding aspect, wherein the infectious disease comprises a parasitic infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

Also disclosed herein are methods of increasing the number of monocyte-derived dendritic cells (moDCs) of any preceding aspect, further comprising detecting whether the cancer is highly immunosuppressive prior to administration of the fucose.

In one aspect, disclosed herein are methods increasing antigen presentation by monocyte derived dendritic cells in a tumor and/or infectious microenvironment, local site of an antigen from a vaccine, or the draining lymph node of a vaccine, said method comprising administering to the subject with a cancer (such as, for example, tumor microenvironment of a melanoma or breast cancer) or infection or the subject receiving a vaccine, an agent that increases the amount of fucosylation on monocyte derived dendritic cells (such as, for example, a fucose including, but not limited to L-fucose, D-fucose, fucose-1-phosphate, or GDP-L-fucose); wherein the increase in fucosylation causes an increase in the number and length of dendrites on the dendritic cell thereby increasing antigen presentation. In one aspect the fucose is not the fucosylation inhibitor 2-fluoro-fucose (2FF). In some aspects, the method can further comprise administering to the subject an autologous dendritic cell.

Also disclosed herein are methods increasing antigen presentation by monocyte derived dendritic cells of any preceding aspect, wherein the antigen is a viral antigen from a virus selected from the group of viruses consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In one aspect disclosed herein are methods increasing antigen presentation by monocyte derived dendritic cells of any preceding aspect, wherein the antigen is a bacterial antigen from a bacteria selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

Also disclosed herein are methods increasing antigen presentation by monocyte derived dendritic cells of any preceding aspect, wherein the antigen is a fungal antigen from a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

In one aspect disclosed herein are methods increasing antigen presentation by monocyte derived dendritic cells of any preceding aspect, wherein the antigen is from a parasitic infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

Also disclosed herein are methods increasing antigen presentation by monocyte derived dendritic cells of any preceding aspect, wherein the antigen is produced by a vaccine (such as, for example, an mRNA, peptide, protein, heat killed infectious agent, or live attenuated infectious agent).

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, 1H, 1I, 1J, 1K, 1L, 1M, 1N, and 1O show that increasing melanoma fucosylation reduces tumor growth and increases itIC abundance, particularly CD4⁺ and CD8⁺ T cells. Volumetric growth curves, total itIC counts, % itIC subpopulations (CD3⁺ T cells, dendritic cells (DCs), natural killer cells (NKs), macrophages (MΦ), and MDSC-like (MDSC) cells), and intratumoral CD3⁺/CD4⁺ (CD4⁺) and CD3⁺/CD8⁺ (CD8⁺) T cell counts of SW1 tumors (1a, 1b, 1c, & 1d, respectively) or of empty vector (EV)- or mouse fucokinase (mFUK)-expressing SW1 tumors (1e, 1f, 1g, & 1h, respectively) in C3H/HeN mice. ▾=initiated L-fucose supplementation. The growth curves are means±SEM from ≥7 mice per group. *=p<0.05. FIG. 1I shows an association of melanoma-specific fucosylation and CD3⁺T cell density (log 2 scale) in a 41-patient melanoma tissue microarray. FIG. 1J shows boxplots showing lower melanoma-specific fucosylation in male than female patients. FIG. 1K shows scatterplots showing higher correlation between melanoma-specific fucosylation and CD3⁺ T cell density (log 2 scale) is higher in male (Spearman's rho=0.43; p=0.036) than female (Spearman's rho=0.25; p=0.3367) patients. Volumetric growth curves for SW1 tumors in (1l) PBS (control)-injected, (1m) CD8⁺T cell-, or (1n) CD4⁺T cell-immunodepleted C3H/HeN mice. FIG. 1) shows a comparison of intratumoral NK, DC, CD8⁺ T, and CD4⁺T cell subpopulations (absolute cell numbers) from tumors in (1l) and (1n).

FIGS. 2A, 2B, 2C, 2D, and 2E show that lymph node egress is necessary for L-fucose-triggered tumor suppression; L-fucose increases intratumoral CD4⁺T stem and central memory cells. FIG. 1A shows immune subpopulations markers use to profile by flow cytometry. FIG. 1B shows volumetric growth curves for SW1 tumors in C3H/HeN mice fed without (Ctl) or with L-fucose (LF) and treated with FTY720 (Ctl mice administered FTY720: (FTY); LF-supplemented mice administered FTY720: (L+F)). FTY720 was administered at 20 μg per mouse every 2 days starting on Day 12, just prior to the initiation of LF. FIG. 1C shows pie charts showing ratios of intratumoral or lymph node-resident CD4⁺ or CD8⁺ T cell subpopulations, as well as DC subtypes from mice at Day 14, 28, and 42 (each pie chart represents 4-5 mice). Assessment of cytotoxic CD4⁺T cell populations (CRTAM⁺) and cytotoxic CD8⁺ T cell populations (GrzB⁺) from tumors at Day 28 (1d) and Day 42 (1e). Corresponding raw flow cytometric data for these charts are shown in Table 1. The tumor growth curves are means±SEM from ≥7 mice per group. *=p<0.05.

FIGS. 3A, 3B, 3C, 3D, 3E, 3F, 3G, and 3H show that HLA-DRB1 is expressed, fucosylated, and required for L-fucose-triggered melanoma suppression and increased TIL abundance. FIG. 1A shows an immunoblot (IB) analysis of HLA-A and HLA-DRB1 levels in primary human melanocytes (HEMN) or indicated human melanoma cell lines. FIG. 1B shows lectin pulldown (LPD) and IB analysis of patient-matched primary and metastatic cell line pairs WM793 and 1205Lu (left) and WM115 and WM266-4 (right) for HLA-A and HLA-DRB1. FIG. 1C shows V5-immunoprecipitation (IP) and IB analyses of WM793 cells expressing (left) V5-tagged HLA-A or (right) V5-tagged HLA-DRB1. Volumetric growth curves for (1d) non-targeting control shRNA (shNT)-, (1e) H2K1-targeting shRNA (shH2K1)-, or (1f) H2EB1-targeting shRNA (shEB1)-expressing SW1 tumors in C3H/HeN mice. Flow cytometric comparison of (1g) total itIC counts or (1h) indicated subpopulations from shNT- or shEB1-expressing tumors in (1d) and (1f). For (1d, 1e, and 1f), ▾=initiated L-fucose supplementation; growth curves are means±SEM from ≥7 mice per group. *=p<0.05.

FIGS. 4A, 4B, 4C, 4D, 4E, and 4F show that N-linked fucosylation of HLA-DRB1 at N48 regulates its cell surface localization and is required for tumor suppression and increased TIL abundance. FIG. 4A shows (upper) Amino acid sequence alignments showing conservation of predicted N- and O-linked fucosylation sites in human HLA-DRB1 (N48 and T129) and mouse H2EB1 (N46 and T147). Structural modeling of the HLA-DRB1:HLA-DM (lower left) and CD4:HLA-DRB1:TCR (lower right) complexes. Potential glycosylation sites, N48 and T129, of HLA-DR1 beta chain are shown as sticks. CD4 (cyan), HLA-DRB1 (yellow), antigen peptide (magenta), and TCR (green)(lower right). FIG. 4B shows HLA-DRB1 peptide fragment identified by nano-LC/MS to be fucosylated on N48, with predicted HexNAc(4)Hex(3)Fuc(1) glycan structure shown above. FIG. 4C shows lectin pull down (LPD) and IB analyses of EV and V5-tagged wild-type HLA-DRB1 (WT)-, HLA-DRB1 N48G (N48G)-, and HLA-DRB1 T129A (T129A)-expressing WM793 cells. FIG. 4D shows DMSO- or fucosyltransferase inhibitor (FUTi)-treated WM793 cells immunofluorescently stained for endogenous HLA-DRB1, KDEL (ER marker), and DAPI (20× magnification). FIG. 4E shows flow cytometric analysis for relative cell surface fucosylation (upper) and cell surface HLA-DRB1 (upper middle), qRT-PCR analysis of relative HLA-DRB1 mRNA levels (lower middle), and IB analysis of HLA-DRB1 protein levels (lower) in WM793 and 1205Lu cells treated with DMSO (D), 250 μM FUTi (i), or 250 μM L-fuc (LF). FIG. 4F shows volumetric growth curves for shNT (non-targeting shRNA)+EV (control SW1 tumors)(upper left) or shEB1 tumors reconstituted with EV (upper right), EB1 WT (lower left), or EB1 N46G (lower right) in C3H/HeN mice. Control (grey) or L-fucose supplemented water (red, 100 mM; ▾=initiated supplementation) was provided ad libitum. The tumor growth curves are means±SEM from ≥7 mice per group. *=p<0.05.

FIGS. 5A and 5B show that administration of combination L-fucose and anti-PD-1 suppresses tumors and increases intratumoral CD4− T central and effector memory cells. FIG. 5A shows volumetric growth curves for SW1 tumors in C3H/HeN mice (left) and SM1 tumors in C57BL/6 mice (right) fed±L-fucose (LF) and treated with PBS (control) or anti-PD-1. (concurrent initiation of LF±anti-PD1 (▾)). The tumor growth curves are means±SEMfrom ≥7 mice per group. FIG. 5B shows volumetric growth curves for SM1 tumors in C57BL/6 mice fed±L-fucose (LF) and treated with PBS (control) or anti-PD-1 (PD-1). (concurrent initiation of LF±PD1 (▾)). The tumor growth curves are means±SEM from ≥7 mice per group. *=p<0.05. At Day 7 (prior to administration of LF or PD1), Day 21 (endpoint for tumors of control-treated mice), Day 31 (endpoint for tumors of LF-treated mice), Day 63 (endpoint for tumors of PD1-treated mice), the primary tumors (Tumor) and draining lymph nodes (LN) of 4-5 mice per treatment group were analyzed by flow cytometry for intratumor levels of CD4⁺ and CD8⁺ T subpopulations (naive/terminal, stem central/central/effector memory) and dendritic cell (DC) subpopulations (cDC1, cDC2, and monocyte-derived DC (moDC)) as in FIG. 2. Proportions of CD4⁺, CD8⁺, and DC subpopulations in each organ at each timepoints are represented by the color-coded pie charts (each pie chart represents 4-5 mice). Absolute numbers of the subpopulations per 10⁶ cells of tumor/tissue homogenate at each timepoint are represented in the color-coded column charts. Corresponding raw flow cytometric data for these charts are shown in Table 2.

FIGS. 6A, 6B, 6C, 6D, 6E, and 6F shows immunofluorescent visualization of fucosylated HLA-DRB1: development of lectin-mediated proximity ligation technique. FIG. 6A shows a schematic of lectin-mediated proximity ligation analysis (L-PLA) using fucosylated HLA-DRB1 (fuco-HLA-DRB1) as an example. We stained for (i) HLA-DRB1 using α-HLA-DRB1 followed by (+) oligo-conjugated PLA secondary and (ii) fucosylated glycan using biotinylated (“B”) AAL lectin followed by α-biotin followed by (−) oligo-conjugated PLA secondary. Ligated PLA oligos were subjected to rolling circle amplification PCR (RCA PCR), giving rise to fluorescent punctae. FIG. 6B shows representative images of secondary antibody-only control (upper) or full L-PLA (lower) staining of endogenous, fucosylated HLA-DRB1 performed on coverslip-grown WM793 cells (with phalloidin and DAPI co-stains). FIG. 6C shows that to further demonstrate that fuco-HLA-DRB1 L-PLA staining is fucosylation species-specific, we performed L-PLA of endogenous, fuco-HLA-DRB1 on WM793 cells treated with DMSO or FUTi (phalloidin and DAPI co-stains). FIG. 6D shows that to demonstrate specificity of individual L-PLA primary antibodies, FFPE melanoma tissue was stained for melanoma marker (MART1+S100 cocktail), AAL-FITC, HLA-DRB1 (white), and DAPI. FIG. 6E shows representative images of secondary antibody-only control (upper) or full L-PLA (lower) staining of endogenous, fucosylated HLA-DRB1 performed on human melanoma specimens (with MART1+S100 (melanoma markers) and DAPI co-stains). FIG. 6F shows FFPE melanoma tissues were subjected to L-PLA HLA-DRB1 staining±500 mM L-fucose wash and subsequent staining with melanoma marker (MART1+S100 cocktail), and DAPI. Total loss of fuco-HLA-DRB1 signal in the +L-fucose wash tissue confirms the fucose-specificity of L-PLA for fuco-HLA-DRB1. Single melanoma marker and fuco-HLA-DRB1 channels are shown in white for clear visualization.

FIGS. 7A, 7B, 7C, and 7D show the clinical implications of melanoma fucosylation and fucosylated HLA-DRB1 for anti-PD1 in melanoma. FIG. 7A shows representative images of anti-PD1-treated Moffitt patient tumors subjected to immunofluorescent staining for the 2 indicated panels of markers. FIG. 7B shows dot plots showing single-cell distribution of (i) total fucosylation (AAL), (ii) total and (iii) fucosylated HLA-DRB1 staining intensities per melanoma cell, and (iv) % CD4⁺T cells (of total cells) within tumors of 2 responder (Pt. 1 & 2) and 2 non-responder (Pt. 3 & 4) Moffitt patients. FIG. 7C shows box plots showing mean tumor cellular (MTC; means derived from single tumor cell intensities) fucosylation (left), total (center) and fucosylated (right; fuco-HLA-DRB1) HLA-DRB1 staining intensities of anti-PD1 responder (R; red dots) and non-responder (NR; blue dots) patients from (upper) Massachusetts General Hospital ((MGH); n=32) or (lower) MD Anderson Cancer Center ((MDACC); n=11). FIG. 7D shows % intratumoral CD4⁺T cells (of total cells) plotted against corresponding average MTC fuco-HLA-DRB1 for each patient in the MGH (upper) and MDACC (lower) cohorts.

FIGS. 8A, 8B, 8C, 8D, 8E, 8F, 8G, 8H, 8I, 8J, 8K, 8L, 8M, 8N, 80, 8P, 8Q, 8R, 8S, 8T, 8U, 8V, and 8W show confirmation of increased tumor fucosylation and TIL counts, splenic immune cell profiles, and correlations between tumor fucosylation and CD3⁺ T cells in female vs. male melanoma patients. Immunofluorescent (IF) staining analysis of SW1 tumor FFPE sections for intratumoral fucosylation (8a). Flow cytometric profiling of intratumoral immune cell (itIC) subpopulations (CD3⁺ T cells, natural killer cells (NKs), macrophages (MO), MDSC-like cells (MDSCs), and dendritic cells (DCs)) in (8b) SW1 tumors and (8c) spleens in control (Ctl)- or L-fuc (LF)-supplemented SW1 tumor-bearing C3H/HeN mice from FIG. 1a. IF staining analysis of SM1 tumor FFPE sections for intratumoral fucosylation (8d). FIG. 8e shows volumetric growth curves, (8f) total TIL counts, (8g) absolute TIL subpopulations, (8h) splenic immune cell profiles, (8i) % TIL subpopulations, (8j) intratumoral CD4⁺ and CD8⁺T cell counts of SM1 tumors in C57BL6 mice. FIG. 8K shows volumetric growth curves of SW1 tumors in NSG mice. (8l) IB analysis confirming mFUK expression in SW1 cells (upper), IF staining analysis of SW1 tumor FFPE sections for intratumoral fucosylation (lower), and (8m) flow cytometric profiling of indicated immune populations in EV- or mFUK-expressing SW1 tumors from Ctl- or LF-supplemented C3H/HeN mice. FIG. 8n shows flow cytometric profiling of splenic CD4⁺T cells in control (PBS-injected) vs. CD4⁺T cell-depleted (CD4(−)) SW1 tumor-bearing C3H/HeN mice supplemented±LF. FIG. 8O shows IF staining profiling of splenic CD8T cells in control vs. CD8⁺ T cell-depleted (CD8(−)) SW1 tumor-bearing C3H/HeN mice. Flow cytometric profiling of (8p) total TIL counts in control vs. CD4(−) SW1 tumor-bearing C3H/HeN mice and (8q) splenic CD4⁺T cells in control vs. CD4(−) SM1 tumor-bearing C57BL6 mice supplemented±LF. Percentages represent % CD4⁺T of total splenic cells. FIG. 8R shows IF profiling of splenic CD8′T cells in control vs. CD8(−) SM1 tumor-bearing C57BL6 mice fed±L-fuc. Volumetric growth curves for SM1 tumors in (8s) control (PBS)-injected, (8t) CD8(−), or (8u) CD4(−) C57BL6 mice. FIG. 8v shows flow cytometric profiling of total TIL counts in control (s) vs. CD4(−) (8u) mice. FIG. 8W shows a comparison of intratumoral NK, DC, CD8⁺ T, and CD4⁺T subpopulations from control or CD4(−) (α-CD4) depleted tumors in (8s) and (8u). For each mouse model: when tumors reached ˜150 mm³, Ctl- or LF-supplemented water (100 mM; ▾=initiated supplementation) was provided ad libitum. The tumor growth curves are means±SEM from ≥7 mice per group. *=p<0.05. Relative fold-changes in splenic CD8⁺ T cells were determined by (total intrasplenic CD8⁺ signal area/total intrasplenic DAPI area) as a measure of relative CD8⁺ T cell abundance/spleen.

FIGS. 9A, 9B, 9C, 9D, 9E and 9F show fucosylation of CD4⁺T cells affects PKA activity and actin polymerization; and the identification of Integrin β5 as a highly fucosylated protein in activated CD4⁺T cells. FIG. 9A shows the top 5 pathways in human CD4⁺T cells that are affected by increased fucosylation identified by Ingenuity Pathway Analysis (Qiagen; pathways were identified from phosphoproteomic analyses of CD3/CD28-activated human PBMC-derived CD4⁺T cells treated±250 μM L-fucose (LF) isolated from 3 independent healthy human donors (D1, D2, D3)). FIG. 9B shows (left) Immunoblot of PKA phosphorylated substrates (top) and Ponceau staining (bottom) of human PBMC-derived, CD3/CD28-activated CD4⁺T cells that were treated ±250 μM LF (for 72h)±10 μM forskolin (Fkn, a PKA agonist; for 6h). (right) Densitometric quantification of selected bands (red dashed boxes) normalized to Ponceau staining. FIG. 9C shows immunoblot of β-actin (top) and Ponceau staining (middle) of human PBMC-derived, CD3/CD28 activated CD4⁺T cells were treated ±250 μM LF±25 mM DTSP crosslinker (x-link). (bottom) Densitometric quantification of high molecular weight β-actin oligomers (red dashed boxes) normalized to Ponceau intensity and then normalized to −LF, −x-link samples. FIG. 9D (i) Coverslip-grown Jurkat cells treated ±250 μM LF (for 72h), fixed, and stained with DAPI and phalloidin-488 for actin cytoskeleton. Scale bar: 25 μm. *: p=0.006; **: p=0.001. (ii) Integrated actin signal densities per cell from 3 biological replicates of Jurkat cells grown/treated as in (i) were measured using FiJi (NIH) and plotted as shown using GraphPad Prism. FIG. 9E shows the top 5 AAL-bound (fucosylated) proteins in human PBMC-derived, CD3/CD28-activated CD4⁺T cells from (9a) that were identified by Ingenuity Pathway Analysis (Qiagen). FIG. 9F shows that of the 5 top hits, we were only able to validate fucosylation of Integrin β5 by LPD analysis of human PBMC-derived, CD3/CD28-activated CD4⁺T cells.

FIGS. 10A and 10B show Fucosylated mass spectrometric analysis and knockdown efficiency of H2K1 and H2EB1. FIG. 10A shows (left) a schematic for proteomic analysis of fucosylated proteins in human WM793 melanoma cells using pLenti-GFP empty vector (EV)-, pLenti-FUK-GFP (o/e)-, or shFUK-expressing WM793 cells. Click chemistry biotinylated-fucosylated proteins that were pulled down using Neutravidin beads from the 6-alkynyl-L-fucose-labeled cells were subjected to LC-MS/MS, and hits were subjected to the indicated filtering scheme followed by Ingenuity Pathway Analysis (Qiagen). (right) Top 20 pathways, plasma membrane- and immune-related proteins identified by Ingenuity Pathway Analysis (Qiagen) to be significantly altered by fucosylation. FIG. 10B shows qRT-PCR analysis confirming knockdown of H2K1 (shH2K1; left) or H2EB1 (shEB1; right) using 2 shRNAs per target compared to control non-targeting (shNT) shRNA. Red arrows indicate the specific shRNA clones used in functional experiments in the remainder of the study.

FIGS. 11A and 11B show that nano-LC/MS spectral identification of fucosylated HLA-DRB1 peptide; and the effects of modulating fucosylation on HLA-DRB1 localization, total protein, and mRNA levels. FIG. 11A shows nano-LC/MS/MS spectra showing fucosylated HLA-DRB1 peptide (arrow). FIG. 11B shows flow cytometric analysis for relative cell surface fucosylation (upper) and cell surface HLA-DRB1 levels (upper middle), qRT-PCR analysis of relative HLA-DRB1 mRNA levels (lower middle), and IB analysis of HLA-DRB1 protein levels (lower) in indicated melanoma cell lines treated with DMSO (D), 250 μM FUTi (i), or 250VM L-fuc (LF). *=p<0.05.

FIGS. 12A, 12B, 12C, 12D, 12E, 12F, 12G, and 12H show proteomic analysis reveal fuco/glycosylation of HLA-DRB1 decreases binding to calnexin; knockdown/reconstitution and fucosylation of EB1 WT and N46G and its effects on TILs in vivo; loss of MHCII is associated with anti-PD1 failure in melanoma patients. FIG. 12A shows IB analysis of 5% input of V5 IP of tagged EV, HLA-DRB1^{WT}, and HLA-DRB1^{N48G} mutant in WM793 melanoma cells. FIG. 12B shows (top) Top 5 pathways that are affected by HLA-DRB1 fuco/glycosylation identified by Ingenuity Pathway Analysis (Qiagen). (bottom) Individual proteins in the Antigen Presentation Pathway identified in the screen. FIG. 12C shows a schematic of proteins identified in the Antigen Presentation & MHC-II Loading Pathway. The schematic was created using BioRender.com. FIG. 12D shows (top) IP of EV, HLA-DRB1^{WT}, and HLA-DRB1^{N48G} and IB analysis of calnexin (short exposure (s.e.), calnexin (intermediate exposure (i.e.), V5, and β-tubulin. (bottom) Quantification of calnexin band intensity to V5 intensity in V5 IP lanes (relative to HLA-DRB^{WT}). FIG. 12E shows (upper) IB analysis of non-targeting shRNA+empty vector (shNT+EV) or shEB1-expressing cells (frori Extended Data FIG. 3b) reconstituted with FLAG-EV (shEB1+EV), FLAG-EB1^{WT} (shEB1+EB1^{WT}), or FLAG-EB1^{N46G} (shEB1+EB1^{N46G}) (lower) LPD and IB analysis of indicated cells from (upper). FIG. 12F shows total TIL counts and (12g) indicated immune subpopulations in the shNT or shEB1 knockdown/EB1^{WT}- or EB1^{N46G}-reconstituted SW1 tumors of the Ctl- or LF-supplemented C3H/HeN mice. *=p<0.05. FIG. 12H shows % of total CD45−/CD90−/EpCAM− tumor cells exhibiting positive pan MHC-I (left) or pan MHC-II (lower) staining from either anti-PD1 naïve patients (black squares; n=7) or patients who failed anti-PD1 (grey squares; n=13).

FIGS. 13A, 13B, 13C, 13D, and 13E show spatial and pre-/post-anti-PD1 trends in fucosylation, total-/fuco-HLA-DRB1 & CD4⁺T cells in patient specimens, lack of effect of fucosylation on cell surface presence of melanoma PD-L1, & examples of stromal content discrepancy among patient biopsies. (EDF 6) Association of mean tumor cellular (MTC) fucosylated HLA-DRB1 with % CD4⁺T cells either inside (tumor marker (+); upper) or outside (tumor border/periphery; tumor marker (−); lower) melanoma tumors in patients from (13a) Massachusetts General Hospital or (13b) MD Anderson Cancer Center (MDACC). FIG. 13C shows mean tumor cellular (MTC) total fucosylation (upper), total HLA-DRB1 (middle), or fucosylated HLA-DRB1 (lower) levels in MDACC patient-matched pre-/post-anti-PD1 tumor specimens. C/P/N=Complete/partial/non-responder, respectively. FIG. 13D shows WM793 (left), SW1 (middle), and SM1 (right) cells were treated with DMSO (D) or 250 μM FUTi (i) or L-fuc (LF) and subjected to flow cytometric assessment of changes in cell surface levels of fucosylation (upper) and PD-L1 (lower). *=p<0.05. FIG. 13E shows a “Highly correlated” anti-PD1 responder biopsy with high fuco-HLA-DRB1 and CD4⁺T cell (upper) vs. a “non-correlated” responder biopsy with low fuco-HLA-DRB1 and CD4⁺T cells were stained for indicated markers. Yellow dashed lines represent the tumor:stromal interface surrounding melanoma marker-negative stroma in the highly correlated responder that is absent in the non-correlated responder. Yellow asterisks indicate non-nucleated non-specific staining on the non-correlated responder slide.

FIGS. 14A, 14B, 14C, 14D, and 14E show that melanoma cells express androgen-inducible and transcriptionally active AR. FIG. 14A shows AR expression in male and female melanoma tissues from TCGA skin cutaneous melanoma (SKCM) dataset. FIG. 14B shows AR expression in primary and metastatic melanomas from TCGA SKCM dataset. FIG. 14C shows immunoblotting analysis of baseline AR protein level across 10 melanoma cell lines. FIG. 14D shows nuclear fractionation followed by immunoblotting of AR protein in WM793 cells±100 nM dihydrotestosterone (DHT) over 96 hours. FIG. 14E shows AR binding motif-containing promoter (ARR2) luciferase assay on WM793 cells±100 nM DHT.

FIGS. 15A, 15B, 15C, and 15D show the biological functions of androgen in melanoma. FIGS. 15A-15C shows (15A) MTT, (15B) BrdU, and (15C) Wound healing assays for WM793 cells±100 nM DHT. FIG. 15D shows the fold change of tumor volume in C57BL/6-SM1 mice model at the end point (35d after implantation). Mice were castrated 1.5 weeks prior to injection.

FIGS. 16A, 16B, 16C, and 16D show that AR transcriptionally upregulates FUT4 expression via binding to the ARE motif in FUT4 promoter. FIG. 16A shows the predicted AR-binding sites in the promoter of FUT4 (SEQ ID NO: 21), FUT1 (SEQ ID NO: 22), SLC35C2 (SEQ ID NO: 23), and FUK (SEQ ID NO: 24) genes. FIG. 16B shows qRT-PCR assessing mRNA levels of FUK and FUT4 altered by DHT treatment in WM793 cells. FIG. 16C shows ChIPqPCR analysis of the enrichment of AR protein at −515-502 bp promoter region of FUT4 gene upon DHT treatment. FIG. 16D shows hallmark GSEA associates FUT4 expression with androgen response gene signatures in TCGA SKCM samples.

FIGS. 17A and 17B show AR-FUT4-dependent signaling regulates cell adhesion/motility, whereas AR-dependent/FUT4-independent signaling regulates cell division. FIG. 17A shows phosphoproteomics profiling of EV/FUT4-OE melanoma cells±AR inhibitor. Pathway enrichment analyses were performed on DAVID (Functional Annotation Tool). FIG. 17B shows ingenuity pathway analysis (IPA) listed adherens junctions (AJs) as the top 1 AR/FUT4-regulated signaling.

FIGS. 18A, 18B, 18C, 18D, and 18E show AR-FUT4 axis facilitates melanoma invasion via disrupting N-cadherin/catenin junction complexes. FIG. 18A shows clonogenic assay on WM793 cells±10 uM AR inhibitor or ±cultured in charcoal-stripped serum (CSS). FIG. 18B shows wound healing assay, (18C) Matrigel invasion assay, and (18D) 3D spheroid cell invasion assay on EV/FUT4-OE WM793 cells±10 uM AR inhibitor. FIG. 18E shows proximity ligation assay evaluating the interaction of N-cadherin and β-catenin proteins in EV/FUT4-OE WM793 cells and parental WM793 cells±10 uM AR inhibitor.

FIGS. 19A, 19B, 19C, and 19D show FUT4-fucosylated LICAM is required for AR-FUT4-induced melanoma invasiveness. FIG. 19A shows fucoproteomics profiling of WM793 cells ectopically expressing FUT4. FIG. 19B shows GeneMania interactome mapping of eight protein hits. FIG. 19C shows lectin proximity ligation assay on EV/FUT4-OE and shNT/shFUT4 WM793 cells. FIG. 19D shows matrigel invasion assay on FUT4 and LICAM double-modified WM793 cells.

FIGS. 20A, 20B, 20C, and 20D show the activation of AR-FUT4-L1CAM-AJs signaling axis in male melanomas. FIG. 20A shows representative pictures of multiplexed immunofluorescence-stained melanoma TMA (#ME1004h). FIG. 20B shows (left) The level of relative activated AR (the ratio of nuclear AR/cytoplasmic AR) between female and male melanomas. (right) The level of activated AR in ARhigh melanoma cell population between primary and metastatic melanomas. FIG. 20C shows the Correlation analysis of activated AR & fucosylated-LICAM (LPLA Foci) as well as (20D) of activated AR & N-Cad/β-catenin junction complexes (PLA Foci).

FIG. 21A shows L-fucose treatment of breast tumors leads to dose-dependent tumor suppression. To determine the effect of L-fucose treatment on a less immunologically active tumor we used a syngeneic breast tumor model and measured the dose-dependent tumor suppression.

FIGS. 21B and 21C show L-fucose treated breast tumors show an enrichment of CD11c+ cells. To determine which immune populations may be contributing to the reduced tumor growth we next harvested the tumors and performed flow cytometry to identify common subsets of immune cells (T cells, NK cells, DCs and macrophages (21B) we then compared the change between the 500 mM L-fucose treated group to the control group to determine which populations had the highest overall change (21C).

FIGS. 22A, 22B, and 22C show Modulation of BMMC fucosylation alters DC immunostimulatory capacity ex vivo. To assess whether the effect in DC enrichment after L-fucose resulted in altered DC biology we isolated immature myeloid cells from BM to (22A) confirm that CD11c+ population was enriched. We next sought to see how the immunostimulatory capacity of these cells was altered after L-fucose treatment by culturing L-fucose modulated, either by decreased fucosylation via 2FF or increased fucosylation by L-fuc, MCs with immature T cells to asses (22B) proliferation by loss of CTV staining and (22C) T cell activation as evident by interferon-gamma release.

FIG. 23A shows L-fucose treatment leads to DC polarization of BMMC at any stage of myeloid development. To test whether L-fucose affected immature or mature myeloid cells preferentially, BMMC were treated with L-fucose at various stages of development, pre=before maturation cocktail, concurrent=during maturation cocktail or post=after maturation cocktail.

FIG. 23B shows L-fucose polarizes myeloid cells towards a moDC phenotype. We next sought to determine if L-fucose equally affected subpopulations of DCs. By flow cytometric analysis we were able to determine that moDC are affected most by the modulation of fucosylation in immature myeloid cell populations.

FIGS. 24A and 24B show L-fucose increases expression of CD209 and modulates downstream signaling paths

FIGS. 25A and 25B shows that DCs treated with L-fucose show decreased immunosuppressive signature. To determine if the changes in DC polarization and CD209 expression were associated with differential signaling changes in moDCs we analyzed known suppressive and stimulatory pathways by (25A) cytokine production and (25B) qPCR. Together these data suggest a loss of immunosuppressive activity and enhanced immunostimulation.

FIG. 25C shows changes in L-fucose treated moDC signaling is associated with decreased iNOS and p65 activation. (C) To elucidate the mechanism by which L-fucose was repressing immunosuppression in moDCs we preformed immunoblot analysis of several key signaling pathways in moDC activation of cells treated +/−L-fucose and +/−maturation via LPS. Additionally, we identified transcription factors that may play a role in altering the signaling and cytokine profiles of moDC to further validate the changes in immunomodulatory activity we observe.

FIGS. 26A and 26B show that moDCs treated with L-fucose exhibit increased dendrite length and phagocytosis. Having determined that L-fucose alters the signaling of moDCs leading to an immunostimulatory phenotype, we next sought to identify other components of DC biology that are affected. To this end we examined L-fucose-treated mature dendritic cells for changes in (A) dendrite length and (B) phagocytosis by neutral bead uptake which may correlate with differential anti-tumor capacity in the TME.

FIGS. 26C and 26D show that moDCs exhibits a cytotoxic-like phenotype after L-fucose treatment. We next sought to determine if the change in phagocytosis we observe in the bead uptake assay was accompanied with increased cytotoxic tendencies in the L-fucose-treated moDCs. To this end we co-cultured pretreated moDCs with GFP-expressing SW1 tumor cells to observe (26C) loss of GFP fluorescence correlating with SW1 and (26D) release of LDH as markers of cell death.

FIG. 26E shows L-fucose treatment is associated with increased clustering in vivo. We next observed SW1 tumors from mice treated +/−L-fucose had increased DC clustering by CD11c (in green).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS AND COMPOSITIONS

Myeloid derived suppressor cells (MDSCs) contribute significantly to immunosuppressive tumor microenvironment, reducing anti-tumor immunity and immunotherapy efficacy. They also contribute to immunosuppression in other diseases; the ability to shut off the immunosuppressive capacity of MDSCs is highly clinically relevant in cancer and other pathologies. We show herein, that L-fucose supplementation (including dietary L-fucose) increases proliferation of MDSC/MDSC-like cells in tumors. These MDSC/MDSC-like cells exhibit significantly reduced immunosuppressive capacity. Instead, these cells elicit immunostimulatory activity (at this point in terms of augmenting T cell activation). It is understood and herein contemplated the myeloid fate of MDSC is not a terminal state and that MDSC can revert to a less differentiated state such as monocytic/dendritic progenitor cells (MDP) and progress to become dendritic cells or macrophage. As shown herein, an agent that increases fucosylation (such as, for example, L-fucose (including, but not limited to L-fucose supplementation (including dietary L-fucose)), D-fucose, fucose-1-phosphate, or GDP-L-fucose) can reprogram maturation of MDP to become dendritic cells or macrophage rather than MDSC. Additionally, an agent that increases fucosylation (such as, for example, L-fucose (including, but not limited to L-fucose supplementation (including dietary L-fucose)), D-fucose, fucose-1-phosphate, or GDP-L-fucose) can reprogram MDSC to a less differentiated state (such as, for example MDP). Thus, in one aspect, disclosed herein are methods of decreasing the number of MDSC in a tumor or infectious microenvironment and/or increasing the number of dendritic cells in a tumor and/or infectious microenvironment comprising administering to the subject an agent (such as, for example, L-fucose (including, but not limited to L-fucose supplementation (including dietary L-fucose)), D-fucose, fucose-1-phosphate, or GDP-L-fucose) that increases the amount of fucosylation on myeloid derived suppressor cells (MDSC) and MDSC-like cells.

Fucosylation, the post-translational modification of proteins with the dietary sugar L-fucose, is a mechanism that is well established for its importance in immune cell biology and organ developmental processes but that is poorly understood in terms of its roles in cancer. Fucose is transported extracellularly through the plasma membrane, where it is first phosphorylated by fucokinase (FUK). Then it is conjugated with GDP, yielding GDP-Fucose, which is a usable form in the cell. GDP-Fucose is transported into the ER/Golgi through SLC35C/2, where it can be conjugated to a serine/threonine via an oxygen, which is referred to as O′-linked fucosylation, or to an arginine via a nitrogen, which is referred to as N′-linked fucosylation. The fucosylated protein can then be either trafficked to the cytoplasm or the cell surface. Global fucosylation is reduced during progression in human melanomas (UEA1 fucose-binding lectin staining analysis of tumor microarray (TMA; n=˜300 patients)) via an ATF2-mediated transcriptional repression of fucokinase (FUK). Importantly, increasing fucosylation by genetic manipulation of tumor cells or by dietary L-fucose supplementation significantly blocks tumor growth and metastasis by >50% in mouse models. The studies herein demonstrate that i) tumor fucosylation levels can be used to identify different stages of cancer, and ii), the manipulation of fucosylation represents a feasible anti-cancer approach.

In one aspect, disclosed herein are methods of increasing the number of monocyte-derived dendritic cells (moDCs) at least 2, 3, 4, 5, 6, 7, 8, 9, 10, 11, 12, 13, 14, 15, 16, 17, 18, 19, 20, 21, 22, 23, 24, 25, 30, 35, 40, 45, or 50-fold (for example between about 10-fold and about 50-fold) in a tumor microenvironment (such as, for example, tumor microenvironment of a melanoma or breast cancer) or site of infection of an infectious disease in a subject comprising administering to the subject an agent that increases the amount of fucosylation on myeloid cells (such as, for example, a fucose including, but not limited to L-fucose, D-fucose, fucose-1-phosphate, or GDP-L-fucose). In one aspect the fucose is not the fucosylation inhibitor 2-fluoro-fucose (2FF). In some aspects, the method can further comprise administering to the subject an autologous dendritic cell.

Also disclosed herein are methods of increasing the number of monocyte-derived dendritic cells (moDCs), further comprising administering to the subject an immune checkpoint blockade inhibitor (such as, for example, PD-1 inhibitors lambrolizumab, OPDIVO® (Nivolumab), KEYTRUDA® (pembrolizumab), and/or pidilizumab; the PD-L1 inhibitors BMS-936559, TECENTRIQ® (Atezolizumab), IMFINZI® (Durvalumab), and/or BAVENCIO® (Avelumab); and/or the CTLA-4 inhibitor YERVOY (ipilimumab)). In one aspect, the fucose increasing agent is administered before and/or contiguous with administration of the immune checkpoint inhibitor.

In one aspect, disclosed herein are methods of increasing the number of monocyte-derived dendritic cells (moDCs), further comprising administering to the subject an adoptive cell therapy (such as, for example the transfer of tumor infiltrating lymphocytes (TILs), tumor infiltrating NK cells (TINKs), dendritic cell (DC), marrow infiltrating lymphocytes (MILs), chimeric antigen receptor (CAR) T cells, and/or CAR NK cells).

Also disclosed herein are methods of increasing the number of monocyte-derived dendritic cells (moDCs), wherein the infectious disease comprises an infection from a virus selected from the group of viruses consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In one aspect, disclosed herein are methods of increasing the number of monocyte-derived dendritic cells (moDCs), wherein the infectious disease comprises an infection from a bacteria selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

Also disclosed herein are methods of increasing the number of monocyte-derived dendritic cells (moDCs), wherein the infectious disease comprises an infection from a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Altenaria alternata.

In one aspect, disclosed herein are methods of increasing the number of monocyte-derived dendritic cells (moDCs), wherein the infectious disease comprises a parasitic infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

Also disclosed herein are methods of increasing the number of monocyte-derived dendritic cells (moDCs), further comprising detecting whether the cancer is highly immunosuppressive prior to administration of the fucose.

In one aspect, disclosed herein are methods increasing antigen presentation at least 2, 3, 4, 5, 6, 7, 8, 9, 10, 11, 12, 13, 14, 15, 16, 17, 18, 19, 20, 21, 22, 23, 24, 25, 30, 35, 40, 45, or 50-fold (for example between about 10-fold and about 50-fold) by monocyte derived dendritic cells in a tumor and/or infectious microenvironment, local site of an antigen from a vaccine, or the draining lymph node of a vaccine, said method comprising administering to the subject with a cancer (such as, for example, tumor microenvironment of a melanoma or breast cancer) or infection or the subject receiving a vaccine, an agent that increases the amount of fucosylation on monocyte derived dendritic cells (such as, for example, a fucose including, but not limited to L-fucose, D-fucose, fucose-1-phosphate, or GDP-L-fucose); wherein the increase in fucosylation causes an increase in the number and length of dendrites on the dendritic cell thereby increasing antigen presentation. In one aspect the fucose is not the fucosylation inhibitor 2-fluoro-fucose (2FF). In some aspects, the method can further comprise administering to the subject an autologous dendritic cell.

Also disclosed herein are methods increasing antigen presentation by monocyte derived dendritic cells, wherein the antigen is a viral antigen from a virus selected from the group of viruses consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In one aspect disclosed herein are methods increasing antigen presentation by monocyte derived dendritic cells, wherein the antigen is a bacterial antigen from a bacteria selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

Also disclosed herein are methods increasing antigen presentation by monocyte derived dendritic cells, wherein the antigen is a fungal antigen from a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

In one aspect disclosed herein are methods increasing antigen presentation by monocyte derived dendritic cells, wherein the antigen is from a parasitic infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

Also disclosed herein are methods increasing antigen presentation by monocyte derived dendritic cells, wherein the antigen is produced by a vaccine (such as, for example, an mRNA, peptide, protein, heat killed infectious agent, or live attenuated infectious agent).

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the fucose comprising compositions into the nose and nasal passages through one or both of the nares and can comprise delivery by a spraying mechanism or droplet mechanism, or through aerosolization of the nucleic acid or vector. Administration of the fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) by inhalant can be through the nose or mouth via delivery by a spraying or droplet mechanism. Delivery can also be directly to any area of the respiratory system (e.g., lungs) via intubation. The exact amount of the fucose comprising compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions), if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The fucose comprising compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions for oral administration include powders or granules, suspensions or solutions in water or non-aqueous media, capsules, sachets, or tablets. Thickeners, flavorings, diluents, emulsifiers, dispersing aids or binders may be desirable.

Some of the fucose modulating compositions (including, but not limited to fucose (such as, for example L-fucose, D-fucose, fucose-1-phosphate, or GDP-L-fucose) and fucose comprising compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the fucose comprising compositions may be determined empirically, and making such determinations is within the skill in the art. The dosage ranges for the administration of the fucose comprising compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In the methods disclosed herein, the fucose can be administered before, after, and/or during administration of the immune checkpoint inhibitor. When administered before or after administration of an immune checkpoint inhibitor, administration occurs at least 1, 2, 3, 4, 5, 10, 15, 30, 45 minutes, 1, 2, 3, 4, 5, 6, 7, 8, 9, 10, 11, 12, 18, 24, 36, 48, 60, 72, 84, or 96 hours before or after administration of an immune checkpoint inhibitor. Similarly, where the fucose is administered to increase antigen presentation by monocyte derived dendritic cells in a tumor and/or infectious microenvironment, local site of an antigen from a vaccine, or the draining lymph node of a vaccine, administration of the fucose can occur before, after, and/or during administration of the antigen and/or vaccine. When administered before or after administration of an immune checkpoint inhibitor, administration occurs at least 1, 2, 3, 4, 5, 10, 15, 30, 45 minutes, 1, 2, 3, 4, 5, 6, 7, 8, 9, 10, 11, 12, 18, 24, 36, 48, 60, 72, 84, or 96 hours before or after administration of the antigen and/or vaccine.

As noted above, the disclosed compositions and methods can be used to treat any disease where uncontrolled cellular proliferation occurs such as cancers. A representative but non-limiting list of cancers that the disclosed compositions can be used to treat is the following: lyinphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride , Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pemibroliurnab, CT-011, MK-3475), PD-L1 (such as, for example, atezolizumab, avelurnab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lynphocyte attenuator (BTLA), V-dornain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: MoDC Enrichment with Fucose that Lead to a DC Vaccine

Immunotherapy responsiveness can be impaired by insufficient abundance and activity of tumor-infiltrating lymphocytes (TILs). Elucidating TIL biology and developing safe and effective strategies to increase TILs are crucial for improving the efficacy of immunotherapies.

Fucosylation, the conjugation of glycoproteins with the sugar L-fucose (L-fuc) at asparagine or serine/threonine residues (N- or O-linked, respectively) is mediated by 13 fucosyltransferases (FUTs) and impacts protein functions that are crucial for immune and developmental processes. Whereas altered fucosylation has been reported in a number of cancers, our understanding of its mechanisms and functional contributions is limited. We found that global fucosylation decreases during melanoma progression, and increased tumor fucosylation levels correlate with favorable patient survival outcomes. Further, increasing melanoma fucosylation in a syngeneic mouse model reduced tumor growth and metastasis and significantly increased intratumoral immune cells (itICs). How fucosylation regulates anti-tumor immunity, however, was unknown. Here, we report for the first time that dietary L-fuc can regulate the biology and interactions between CD4⁺T and melanoma cells, robustly inducing TILs and anti-melanoma immunity. Our findings demonstrate the ability of L-fuc to improve the efficacy of immunotherapies and identify novel fucosylation-based biomarkers that can enhance patient stratification.

### a) Results

(1) Increasing Melanoma Fucosylation Impairs Tumor Growth and augmentsTIL Abundance, Particularly CD4⁺ and CD8⁺ T Cells

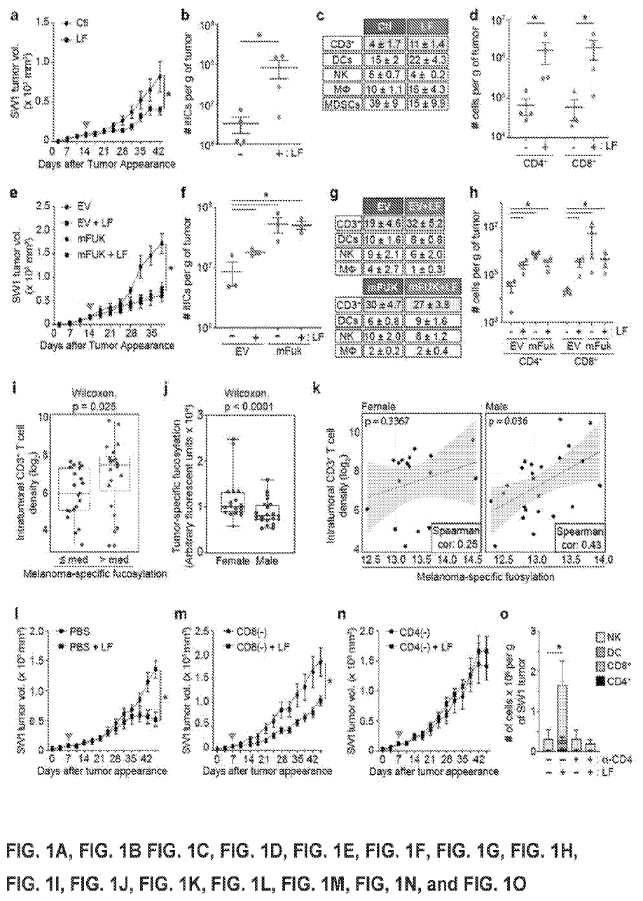

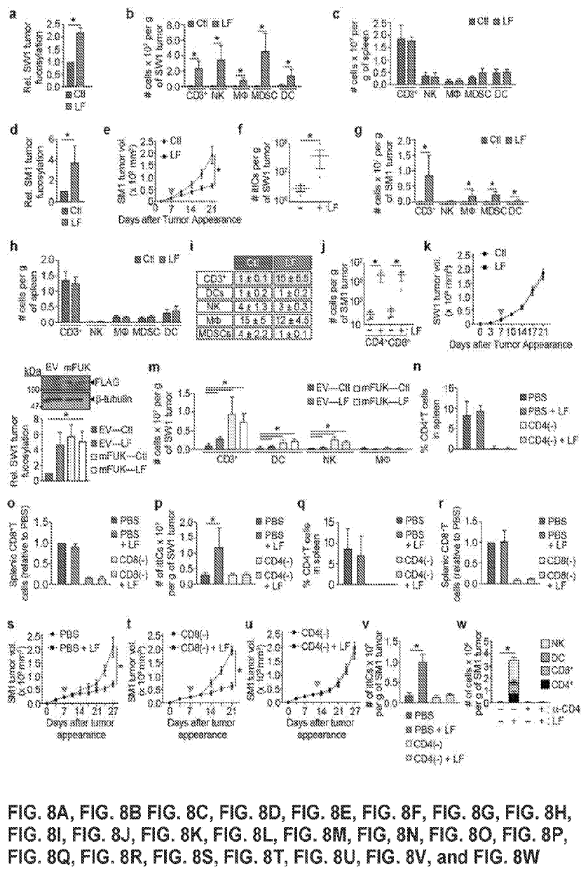

We initially assessed how L-fuc-induced changes in itICs can contribute to melanoma suppression using a NRAS^{GI3D}-mutant mouse melanoma (SW1) model. Oral L-fuc administration increased tumor fucosylation (˜2-fold), reduced tumor growth (˜50%), and increased total itICs (˜10-50-fold) (including CD3⁺ (CD4⁺ and CD8⁺) T, natural killer (NKs), macrophage (MΦ), dendritic (DCs), and myeloid-derived suppressor (MDSCs)-like cell subpopulations, without altering splenic lymphocyte profiles) (FIG. 8a, FIGS. 1a, 1b and FIG. 8b, 8c, respectively). Of total itICs, CD4⁺ and CD8⁺ T cells were the most increased subpopulation (˜doubled) (FIG. 1c, 1d). Oral L-fuc induced similar changes in tumor fucosylation, growth, and TILs—specifically increased CD4⁺ and CD8⁺ T cells—in a BRAF^{V600E}-mutant mouse melanoma (SM1) model (FIG. 8d-8j, respectively). In contrast, L-fuc did not reduce SW1 tumor growth in immunodeficient mice (FIG. 8k), confirming that the presence and activity of itICs are essential for L-fuc-triggered tumor suppression.

We confirmed an essential role for tumor-specific fucosylation by overexpressing murine fucokinase (mFuk) in SW1 melanoma cells to exclusively increase tumor fucosylation. mFuk expression alone suppressed tumor growth and increased total itICs comparably to oral L-fuc alone. Again, CD4⁺ and CD8⁺ T cells were the most increased itICs (FIG. 8l, 8m and FIG. 1e-1h). These data indicate that melanoma-specific fucosylation is an essential determinant of L-fuc-triggered itIC induction and tumor suppression, regardless of any other physiological host effects that L-fuc can elicit (e.g., microbiome or metabolic).

Correlations between tumor fucosylation and CD3⁺ T cells in humans by were assessed immunofluorescently analyzing a 42-patient melanoma microarray. Patients with higher than median tumor fucosylation levels exhibited significantly increased intratumoral CD3⁺ T cell densities (FIG. 1i), even after adjusting for confounding factors including age, sex, and stage (multivariable linear regression). Intriguingly, average melanoma fucosylation levels were lower in male patients (FIG. 1j) but exhibited a stronger association with intratumoral CD3⁺ T cells (FIG. 1k).

These data indicate that melanoma fucosylation significantly shapes itIC landscape, correlates with increased intratumoral CD3⁺ T cells in mice and humans, and can be boosted by oral L-fuc to increase TILs and suppress BRAF- and NRAS-mutant melanomas.

(2) L-Fucose Suppresses Melanomas by Triggering CD4⁺T Cell-Mediated Increases in ITICs and Altering CD4⁺T Cell Biology, Increasing Memory CD4⁺T Subpopulations

The contribution of CD4⁺ and CD8⁺ T cells to L-fuc-triggered tumor suppression was assessed by immunodepletion in the SW1 model. L-fucose reduced tumor growth by >50% in control and CD8⁺ T cell-depleted mice, whereas this effect was completely abrogated by CD4⁺T cell depletion (FIG. 1l-n; immunodepletion confirmed by splenic profiling, FIGS. 8n and 8o). Consistent with known roles for CD4⁺T cells in recruiting and activating tumor suppressive TILs, CD4⁺T cell-depletion also blocked L-fuc-induced increases in total itICs, including intratumoral NK, DC, and CD8⁺ T cells, observed in control mice (FIG. 8p and FIG. 1o). Similarly, in the SM1 model, CD4⁺ but not CD8⁺ T cell depletion abrogated L-fuc-triggered tumor suppression and increases in total itICs and itIC subpopulations (immunodepletion confirmed by splenic profiling, FIG. 8q-8w)).

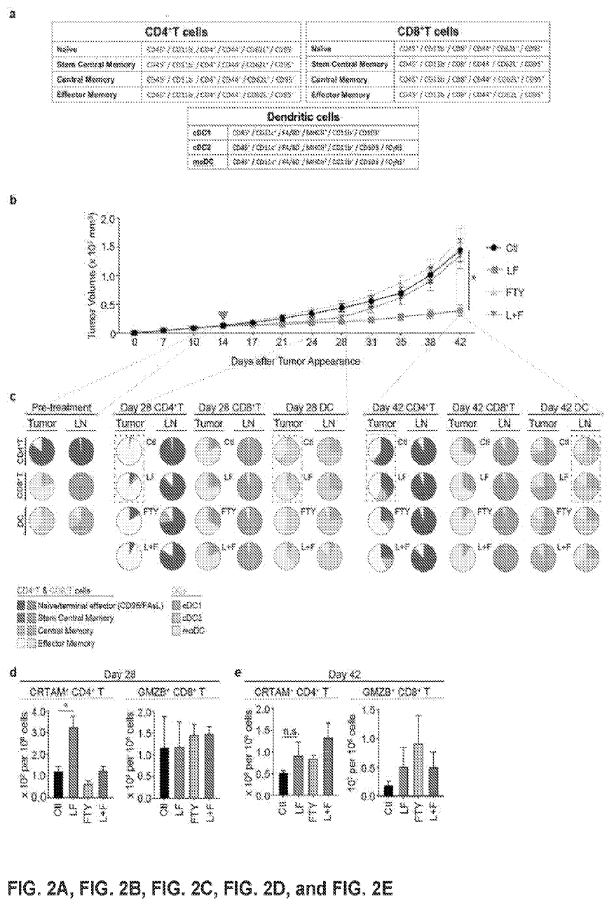

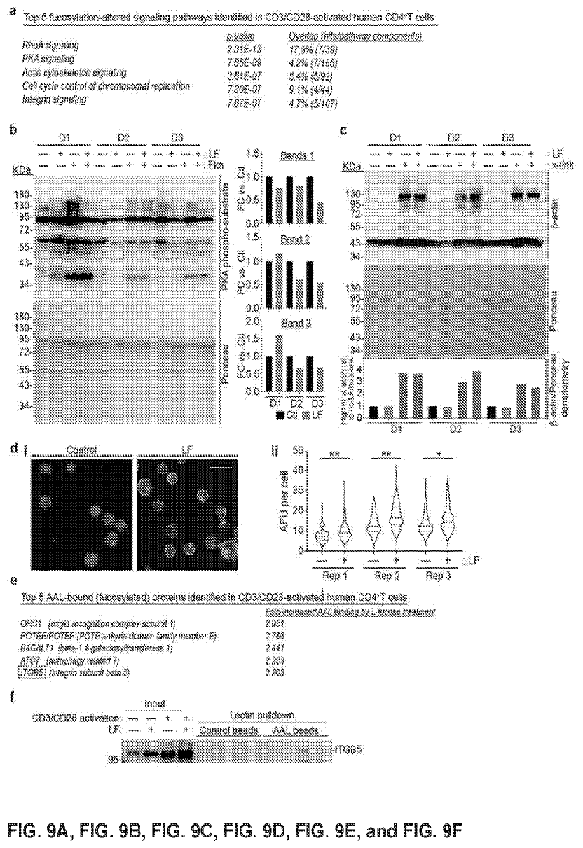

Phosphoproteomic and fucosylated proteomic analyses revealed that L-fuc mechanistically regulates CD4⁺T cell biology by significantly altering Protein Kinase A (PKA) and (to a lesser extent) actin signaling, potentially via Integrin B5, an upstream regulator of both of these pathways that we discovered to be 1 of 5 proteins most highly bound to AAL (and likely fucosylated) in human peripheral blood monocyte (PBMC)-derived, CD3/CD28-activated CD4⁺T cells, as well as Jurkat cells treated with L-fuc (FIG. 9a-9f). That integrin, PKA, and actin signaling have been reported to mediate T cell activation, motility, and immune synapse formation indicates that L-fuc promotes T cell trafficking to the tumor, a notion confirmed using a SW1 melanoma C3H mouse model treated ±FTY720 (an inhibitor of lymph node egress). Inhibition of lymph node egress completely abrogated L-fuc-triggered tumor suppression (FIG. 2a, 2b). Strikingly, L-fuc-triggered tumor suppression was associated with increases in intratumoral CD4⁺T central and effector memory subpopulations that were abrogated by FTY720 (FIG. 2a, 2c (blue dashed boxes) and Table 1), consistent with the role that PKA plays in regulating memory phenotype in T cells. Intriguingly, oral L-fuc induced significant, albeit transient, increases in intratumoral monocyte-derived DCs (moDCs) and lymph node cDC2s, which can promote memory CD4⁺T phenotypes and crosstalk with CD4⁺T cells to mediate tumor suppression, respectively (FIG. 2a, 2c (orange dashed boxes) and Table 1). Finally, L-fuc also transiently but significantly increased cytotoxic CD4⁺T cells at the midpoint (Day 28) of the experiment (FIG. 2d, 2e).

TABLE 1
All non-SEM values represent the average # of indicated cell
types per 10\hat{ }6 cells of total tumor homogenate of the indicated
mice (3-5 mice per treatment group).

| | TUMORS | | | | LYMPH NODES | | | |
|---|---|---|---|---|---|---|---|---|
| | Treatment groups | | | | Treatment groups | | | |
| | | | | FTY720 + | | | | FTY720 + |
| | Control | L-fucose | FTY720 | L-fucose | Control | L-fucose | FTY720 | L-fucose |
| Day 12 | | | | | | | | |
| CD4 naïve | 5169.21 | | | | 127688.76 | | | |
| SEM | 5109.84 | | | | 1582.02 | | | |
| CD4 stem central memory | 82.39 | | | | 1100.52 | | | |
| SEM | 79.20 | | | | 162.28 | | | |
| CD4 central memory | 95.98 | | | | 483.89 | | | |
| SEM | 58.91 | | | | 89.49 | | | |
| CD4 effector memory | 913.81 | | | | 3523.94 | | | |
| SEM | 459.69 | | | | 359.31 | | | |
| CD8 naïve | 59.99 | | | | 57929.19 | | | |
| SEM | 18.22 | | | | 6304.49 | | | |
| CD8 stem central memory | 9.85 | | | | 906.06 | | | |
| SEM | 3.85 | | | | 277.81 | | | |
| CD8 central memory | 192.92 | | | | 409.50 | | | |
| SEM | 75.19 | | | | 83.15 | | | |
| CD8 effector memory | 954.21 | | | | 601.62 | | | |
| SEM | 523.30 | | | | 211.62 | | | |
| cDC1 | 1034.53 | | | | 456.34 | | | |
| SEM | 654.51 | | | | 209.22 | | | |
| cDC2 | 18855.22 | | | | 135.84 | | | |
| SEM | 3021.94 | | | | 19.14 | | | |
| moDC | 12852.75 | | | | 98.51 | | | |
| SEM | 6492.87 | | | | 95.31 | | | |
| Day 28 | | | | | | | | |
| CD4 naïve | 21.62 | 30.44 | 60.03 | 28.30 | 4030.93 | 9286.89 | 20717.91 | 17023.59 |
| SEM | 6.08 | 7.44 | 13.78 | 4.20 | 1028.55 | 4035.40 | 9884.58 | 8992.20 |
| CTL vs. L-fuc −> P-value | 0.43 | | | | | | | |
| CD4 stem central memory | 4.22 | 5.59 | 0.87 | 0.00 | 268146.09 | 53601.81 | 24893.27 | 18115.92 |
| SEM | 2.45 | 3.95 | 0.78 | 0.00 | 49057.78 | 26129.45 | 13473.70 | 4551.24 |
| CTL vs. L-fuc −> P-value | 0.80 | | | | | | | |
| CD4 central memory | 14.55 | 6.04 | 11.56 | 3.70 | 13175.03 | 2289.20 | 1362.38 | 804.52 |
| SEM | 4.17 | 1.19 | 6.11 | 3.31 | 2311.21 | 1180.74 | 433.53 | 133.56 |
| CTL vs. L-fuc −> P-value | 0.10 | | | | | | | |
| CD4 effector memory | 1268.62 | 568.19 | 336.07 | 381.63 | 1787.69 | 445.15 | 372.53 | 225.43 |
| SEM | 569.70 | 197.62 | 125.47 | 35.90 | 355.45 | 192.32 | 217.07 | 96.05 |
| CTL vs. L-fuc −> P-value | 0.30 | | | | | | | |
| CD8 naïve | 33.04 | 34.60 | 24.54 | 23.14 | 104987.14 | 18072.26 | 6153.14 | 2874.93 |
| SEM | 10.58 | 12.14 | 10.51 | 8.66 | 13217.81 | 7418.79 | 4933.86 | 1056.91 |
| CD8 stem central memory | 0.00 | 0.00 | 0.00 | 0.00 | 6732.04 | 980.96 | 311.59 | 69.43 |
| SEM | 0.00 | 0.00 | 0.00 | 0.00 | 484.47 | 455.62 | 277.81 | 38.27 |
| CD8 central memory | 1.80 | 1.24 | 1.22 | 3.22 | 1487.85 | 295.92 | 85.45 | 50.39 |
| SEM | 2.01 | 1.38 | 1.22 | 2.70 | 189.86 | 165.57 | 82.44 | 26.51 |
| CD8 effector memory | 152.22 | 108.29 | 54.02 | 104.52 | 1266.80 | 888.80 | 421.08 | 74.28 |
| SEM | 67.81 | 53.12 | 10.32 | 17.96 | 355.48 | 366.93 | 378.21 | 40.85 |
| cDC1 | 548.13 | 2301.44 | 463.94 | 2828.33 | 370.65 | 359.10 | 147.79 | 301.65 |
| SEM | 179.92 | 442.53 | 132.10 | 1037.71 | 87.08 | 87.83 | 63.28 | 93.71 |
| CTL vs. L-fuc −> P-value | 0.01 | | | | | | | |
| cDC2 | 11090.62 | 10732.04 | 8181.08 | 9566.75 | 983.17 | 743.69 | 448.71 | 2993.68 |
| SEM | 3201.23 | 3894.78 | 1969.35 | 1870.75 | 86.96 | 83.88 | 168.84 | 860.67 |
| CTL vs. L-fuc −> P-value | 0.94 | | | | | | | |
| moDC | 6796.07 | 20903.90 | 7526.28 | 19154.15 | 25.42 | 37.66 | 41.74 | 487.28 |
| SEM | 1956.32 | 2037.31 | 3093.39 | 4399.04 | 5.80 | 16.29 | 17.41 | 81.53 |
| CTL vs. L-fuc −> P-value | 0.00 | | | | | | | |
| Day 42 | | | | | | | | |
| CD4 naïve | 97.82 | 82.01 | 44.22 | 85.71 | 54819.64 | 105779.76 | 65491.17 | 41632.71 |
| SEM | 18.42 | 36.81 | 7.70 | 29.27 | 31520.40 | 43716.38 | 40555.58 | 11358.92 |
| CTL vs. L-fuc −> P-value | 0.74 | | | | | | | |
| CD4 stem central memory | 12.00 | 16.67 | 18.52 | 20.04 | 954.72 | 1587.46 | 742.12 | 1179.03 |
| SEM | 4.99 | 3.91 | 15.21 | 12.05 | 560.65 | 403.70 | 101.56 | 325.41 |
| CTL vs. L-fuc −> P-value | 0.45 | | | | | | | |
| CD4 central memory | 2.00 | 38.18 | 32.54 | 59.17 | 1025.15 | 1741.12 | 949.35 | 1169.41 |
| SEM | 1.56 | 28.40 | 18.85 | 48.14 | 510.44 | 412.07 | 268.65 | 348.11 |
| CTL vs. L-fuc −> P-value | 0.24 | | | | | | | |
| CD4 effector memory | 69.25 | 111.09 | 160.89 | 219.65 | 4531.66 | 5032.79 | 3499.07 | 5326.18 |
| SEM | 11.07 | 17.84 | 24.60 | 54.43 | 1395.81 | 1023.03 | 623.12 | 960.27 |
| CTL vs. L-fuc −> P-value | 0.01 | | | | | | | |
| CD8 naïve | 61.54 | 109.29 | 19.42 | 63.73 | 54819.64 | 105779.76 | 65491.17 | 41632.71 |
| SEM | 8.79 | 76.21 | 5.88 | 24.99 | 31520.40 | 43716.38 | 40555.58 | 11358.92 |
| CD8 stem central memory | 0.00 | 0.00 | 0.00 | 0.00 | 954.72 | 1587.46 | 742.12 | 1179.03 |
| SEM | 0.00 | 0.00 | 0.00 | 0.00 | 560.65 | 403.70 | 101.56 | 325.41 |
| CD8 central memory | 2.00 | 13.46 | 5.73 | 11.16 | 1025.15 | 1741.12 | 949.35 | 1169.41 |
| SEM | 1.68 | 11.81 | 2.82 | 9.32 | 510.44 | 412.07 | 268.65 | 348.11 |
| CD8 effector memory | 151.90 | 246.44 | 220.02 | 585.79 | 4531.66 | 5032.79 | 3499.07 | 5326.18 |
| SEM | 37.08 | 54.55 | 41.75 | 68.79 | 1395.81 | 1023.03 | 623.12 | 960.27 |
| cDC1 | 871.21 | 648.96 | 607.65 | 2953.95 | 1647.25 | 2141.34 | 1731.49 | 2654.32 |
| SEM | 421.83 | 252.91 | 262.91 | 1200.66 | 602.45 | 681.16 | 698.99 | 762.26 |
| CTL vs. L-fuc −> P-value | | | | | 0.60 | | | |
| cDC2 | 125.49 | 78.44 | 279.84 | 158.37 | 3240.31 | 4375.69 | 3881.72 | 3853.65 |
| SEM | 42.78 | 12.54 | 145.82 | 65.01 | 834.63 | 892.44 | 1415.47 | 301.80 |
| CTL vs. L-fuc −> P-value | | | | | 0.39 | | | |
| moDC | 226.31 | 169.08 | 197.56 | 259.71 | 2197.31 | 1201.44 | 1219.26 | 3230.07 |
| SEM | 50.13 | 57.37 | 33.55 | 72.27 | 872.62 | 363.34 | 569.85 | 2077.67 |
| CTL vs. L-fuc −> P-value | | | | | 0.37 | | | |

TABLE 2
All non-SEM values represent the average # of indicated cell
types per 10\hat{ }6 cells of total tumor homogenate of the indicated
mice (3-5 mice per treatment group).

| | TUMORS | | | | LYMPH NODES | | | |
|---|---|---|---|---|---|---|---|---|
| | Treatment groups | | | | Treatment groups | | | |
| | | | | PD-1 + | | | | PD-1 + |
| | Control | L-fucose | PD-1 | L-fucose | Control | L-fucose | PD-1 | L-fucose |
| Day 7 | | | | | | | | |
| CD4 naïve | 137.43 | | | | 4879.62 | | | |
| SEM | 21.57 | | | | 892.41 | | | |
| CD4 stem central memory | 20.10 | | | | 487.68 | | | |
| SEM | 11.08 | | | | 137.04 | | | |
| CD4 central memory | 10.57 | | | | 248.44 | | | |
| SEM | 1.86 | | | | 120.89 | | | |
| CD4 effector memory | 105.23 | | | | 2853.75 | | | |
| SEM | 17.95 | | | | 1107.08 | | | |
| CD8 naïve | 103.70 | | | | 1493.47 | | | |
| SEM | 1.07 | | | | 558.49 | | | |
| CD8 stem central memory | 2.93 | | | | 446.23 | | | |
| SEM | 0.95 | | | | 310.78 | | | |
| CD8 central memory | 5.29 | | | | 87.92 | | | |
| SEM | 1.23 | | | | 46.22 | | | |
| CD8 effector memory | 106.53 | | | | 517.66 | | | |
| SEM | 20.02 | | | | 301.43 | | | |
| cDC1 | 1312.32 | | | | 23525.87 | | | |
| SEM | 980.07 | | | | 13230.44 | | | |
| cDC2 | 1787.80 | | | | 8689.26 | | | |
| SEM | 160.45 | | | | 6710.52 | | | |
| moDC | 2478.85 | | | | 267.38 | | | |
| SEM | 593.68 | | | | 149.51 | | | |
| Day 21 | | | | | | | | |
| CD4 naïve | 71.54 | 186.42 | 162.94 | 101.73 | 22549.16 | 24832.36 | 15303.62 | 9005.94 |
| SEM | 12.25 | 57.55 | 37.80 | 43.17 | 13528.06 | 9947.16 | 9228.63 | 2565.33 |
| PD-1 vs. PD-1 + L-fuc P value | | | 0.32 | | | | 0.53 | |
| CD4 stem central memory | 1.36 | 3.22 | 1.20 | 0.42 | 905.20 | 512.17 | 503.10 | 155.69 |
| SEM | 0.35 | 1.72 | 0.96 | 0.26 | 652.89 | 221.01 | 213.20 | 53.22 |
| PD-1 vs. PD-1 + L-fuc P value | | | 0.45 | | | | 0.15 | |
| CD4 central memory | 5.39 | 19.50 | 15.05 | 18.90 | 708.24 | 592.12 | 627.49 | 228.34 |
| SEM | 1.25 | 6.24 | 3.06 | 8.55 | 339.24 | 138.35 | 339.37 | 74.77 |
| PD-1 vs. PD-1 + L-fuc P value | | | 0.68 | | | | 0.28 | |
| CD4 effector memory | 132.99 | 302.98 | 231.74 | 118.31 | 1453.08 | 1155.12 | 4211.49 | 449.05 |
| SEM | 24.49 | 121.74 | 77.07 | 35.94 | 639.47 | 359.45 | 3554.80 | 94.98 |
| PD-1 vs. PD-1 + L-fuc P value | | | 0.22 | | | | 0.32 | |
| CD4+ CRTAM+ | 235.64 | 361.60 | 628.54 | 346.35 | | | | |
| SEM | 11.33 | 109.18 | 174.63 | 63.24 | | | | |
| Control vs. L-fuc P-value | | | | | | | | |
| CD8 naïve | 11.99 | 81.09 | 48.82 | 25.85 | 15758.94 | 11896.39 | 5709.62 | 4412.83 |
| SEM | 3.00 | 50.35 | 12.50 | 6.44 | 7973.33 | 4839.03 | 3136.47 | 797.98 |
| PD-1 vs. PD-1 + L-fuc P value | | | | | | | 0.70 | |
| CD8 stem central memory | 0.00 | 1.53 | 0.99 | 0.42 | 482.52 | 192.89 | 360.35 | 64.48 |
| SEM | 0.00 | 1.53 | 0.99 | 0.42 | 455.31 | 111.88 | 283.12 | 43.21 |
| PD-1 vs. PD-1 + L-fuc P value | | | | | | | 0.33 | |
| CD8 central memory | 4.59 | 25.37 | 12.45 | 6.39 | 1865.59 | 1017.54 | 1525.55 | 526.71 |
| SEM | 1.28 | 11.89 | 2.81 | 1.31 | 1125.59 | 126.65 | 977.14 | 277.42 |
| PD-1 vs. PD-1 + L-fuc P value | | | | | | | 0.35 | |
| CD8 effector memory | 224.43 | 889.74 | 869.40 | 264.16 | 1667.71 | 4093.80 | 6959.03 | 2407.46 |
| SEM | 61.35 | 457.67 | 401.20 | 82.67 | 221.85 | 1743.14 | 5631.22 | 440.55 |
| PD-1 vs. PD-1 + L-fuc P value | | | | | | | 0.44 | |
| CD8+ GRZB+ | 3969.71 | 14146.30 | 9842.29 | 5111.75 | | | | |
| SEM | 1221.00 | 10530.55 | 4377.81 | 2001.78 | | | | |
| cDC1 | 1116.43 | 2032.24 | 3243.02 | 2762.65 | 6007.87 | 9395.57 | 7597.50 | 8397.79 |
| SEM | 415.49 | 641.83 | 1681.49 | 1050.26 | 2222.63 | 1890.05 | 1642.58 | 1845.48 |
| PD-1 vs. PD-1 + L-fuc P value | | | | | | | 0.76 | |
| cDC2 | 10248.10 | 12762.45 | 11184.16 | 12553.39 | 9440.63 | 13997.11 | 7819.23 | 9400.56 |
| SEM | 2031.15 | 2486.96 | 3210.61 | 3360.95 | 2431.62 | 3847.20 | 1036.82 | 1462.72 |
| PD-1 vs. PD-1 + L-fuc P value | | | | | | | 0.43 | |
| moDC | 504.70 | 570.99 | 840.14 | 814.84 | 1330.84 | 1788.45 | 7802.04 | 1779.90 |
| SEM | 49.29 | 140.82 | 265.00 | 375.85 | 480.11 | 453.82 | 6446.38 | 741.26 |
| PD-1 vs. PD-1 + L-fuc P value | | | | | | | 0.33 | |
| Day 31 | | | | | | | | |
| CD4 naïve | | 401.23 | 612.31 | 572.86 | | 30715.03 | 66744.66 | 73537.90 |
| SEM | | 102.10 | 147.58 | 138.24 | | 11102.80 | 15766.04 | 12688.24 |
| CD4 stem central memory | | 6.29 | 9.82 | 13.72 | | 408.72 | 815.21 | 1662.26 |
| SEM | | 1.55 | 1.55 | 4.85 | | 175.77 | 159.07 | 433.37 |
| CD4 central memory | | 28.68 | 30.18 | 37.11 | | 424.67 | 301.76 | 726.67 |
| SEM | | 7.64 | 5.98 | 10.46 | | 104.79 | 55.95 | 214.74 |
| CD4 effector memory | | 297.82 | 365.42 | 466.11 | | 6638.79 | 5897.24 | 8927.09 |
| SEM | | 82.52 | 102.42 | 186.20 | | 1286.06 | 1335.86 | 680.38 |
| CD4+ CRTAM+ | | 112.92 | 16.53 | 120.78 | | | | |
| SEM | | 19.79 | 6.99 | 71.48 | | | | |
| CD8 naïve | | 223.96 | 358.26 | 366.90 | | 12415.41 | 20835.63 | 33242.92 |
| SEM | | 62.98 | 96.60 | 77.44 | | 5799.70 | 4829.32 | 5963.32 |
| CD8 stem central memory | | 3.15 | 3.39 | 7.48 | | 56.84 | 35.59 | 119.48 |
| SEM | | 0.36 | 0.37 | 2.27 | | 19.90 | 6.71 | 28.47 |
| CD8 central memory | | 26.40 | 24.59 | 52.52 | | 286.54 | 431.18 | 1025.88 |
| SEM | | 3.06 | 5.59 | 14.55 | | 110.04 | 93.82 | 267.80 |
| CD8 effector memory | | 202.98 | 404.52 | 557.82 | | 2478.29 | 1821.76 | 4641.26 |
| SEM | | 37.38 | 80.45 | 228.80 | | 641.01 | 563.06 | 849.35 |
| CD8+ GRZB+ | | 5679.05 | 11048.81 | 7093.40 | | | | |
| SEM | | 635.34 | 2633.49 | 2321.35 | | | | |
| cDC1 | | 200.82 | 496.71 | 443.81 | | 5781.73 | 5050.57 | 9873.57 |
| SEM | | 27.53 | 286.65 | 159.39 | | 1938.69 | 1415.37 | 1200.24 |
| cDC2 | | 5753.01 | 8162.69 | 5348.55 | | 22628.85 | 28561.93 | 25534.78 |
| SEM | | 1615.48 | 2824.11 | 1079.65 | | 4510.71 | 712.95 | 2500.95 |
| moDC | | 1107.62 | 2609.60 | 1410.55 | | 7315.56 | 9663.42 | 14368.58 |
| SEM | | 293.85 | 1077.67 | 372.54 | | 3944.83 | 1922.37 | 3852.42 |
| Day 63 | | | | | | | | |
| CD4 naïve | | | 44.75 | 279.51 | | | 15958.48 | 33641.37 |
| SEM | | | 28.18 | 84.27 | | | 5482.78 | 7759.20 |
| PD-1 vs. PD-1 + L-fuc P value | | | 0.03 | | | | 0.10 | |
| CD4 stem central memory | | | 0.00 | 8.27 | | | 387.13 | 213.55 |
| SEM | | | 0.00 | 3.95 | | | 146.03 | 40.91 |
| PD-1 vs. PD-1 + L-fuc P value | | | 0.07 | | | | 0.29 | |
| CD4 central memory | | | 10.10 | 87.93 | | | 2649.48 | 1919.79 |
| SEM | | | 2.21 | 17.50 | | | 461.31 | 584.78 |
| PD-1 vs. PD-1 + L-fuc P value | | | 0.00 | | | | 0.36 | |
| CD4 effector memory | | | 209.54 | 961.76 | | | 15369.78 | 8044.90 |
| SEM | | | 107.22 | 286.53 | | | 4729.64 | 2520.06 |
| PD-1 vs. PD-1 + L-fuc P value | | | 0.04 | | | | 0.21 | |
| CD4+ CRTAM+ | | | 222.65 | 850.80 | | | | |
| SEM | | | 47.83 | 287.63 | | | | |
| PD-1 vs. PD-1 + L-fuc P value | | | 0.06 | | | | | |
| CD8 naïve | | | 6.82 | 214.40 | | | 19280.20 | 27964.12 |
| SEM | | | 2.91 | 88.53 | | | 4375.60 | 5391.15 |
| PD-1 vs. PD-1 + L-fuc P value | | | 0.05 | | | | 0.25 | |
| CD8 stem central memory | | | 0.71 | 30.79 | | | 1745.61 | 472.53 |
| SEM | | | 0.71 | 15.51 | | | 717.34 | 165.70 |
| PD-1 vs. PD-1 + L-fuc P value | | | 0.09 | | | | 0.12 | |
| CD8 central memory | | | 10.10 | 87.93 | | | 2225.16 | 583.43 |
| SEM | | | 2.21 | 17.50 | | | 777.06 | 340.78 |
| PD-1 vs. PD-1 + L-fuc P value | | | 0.00 | | | | 0.09 | |
| CD8 effector memory | | | 345.27 | 767.14 | | | 1773.80 | 659.07 |
| SEM | | | 172.18 | 289.80 | | | 737.79 | 258.54 |
| PD-1 vs. PD-1 + L-fuc P value | | | 0.25 | | | | 0.19 | |
| CD8+ GRZB+ | | | 1657.03 | 3303.50 | | | | |
| SEM | | | 876.66 | 2013.14 | | | | |
| cDC1 | | | 1143.25 | 1409.50 | | | 7711.60 | 5055.01 |
| SEM | | | 139.49 | 494.37 | | | 3484.16 | 1954.81 |
| PD-1 vs. PD-1 + L-fuc P value | | | | | | | 0.51 | |
| cDC2 | | | 756.39 | 1809.91 | | | 1268.37 | 1848.41 |
| SEM | | | 300.94 | 489.47 | | | 292.56 | 543.68 |
| PD-1 vs. PD-1 + L-fuc P value | | | | | | | 0.34 | |
| moDC | | | 6040.98 | 7037.74 | | | 1756.93 | 2785.48 |
| SEM | | | 3560.21 | 3128.47 | | | 544.27 | 1754.22 |

| | |
|---|---|
| PD-1 vs. PD-1 + L-fuc P value | 0.55 |

These data confirmed that CD4⁺T cells play a key role in induction of TILs and suppression of melanomas by L-fuc, indicating that L-fuc triggers key changes in CD4⁺T signaling and biology at the tumor and lymph node levels that are important for tumor suppression. Importantly, that mFUK expression alone in melanoma cells resulted in smaller tumors with increased TILs (FIGS. 1e-1h) indicates that melanoma-specific fucosylated protein(s) can also promote anti-tumor immunity, although the mechanism was unclear.

### (3) Fucosylated HLA-DRB1 Mediates L-Fucose-Triggered TIL Induction, Anti-Melanoma Immunity, and Melanoma Suppression

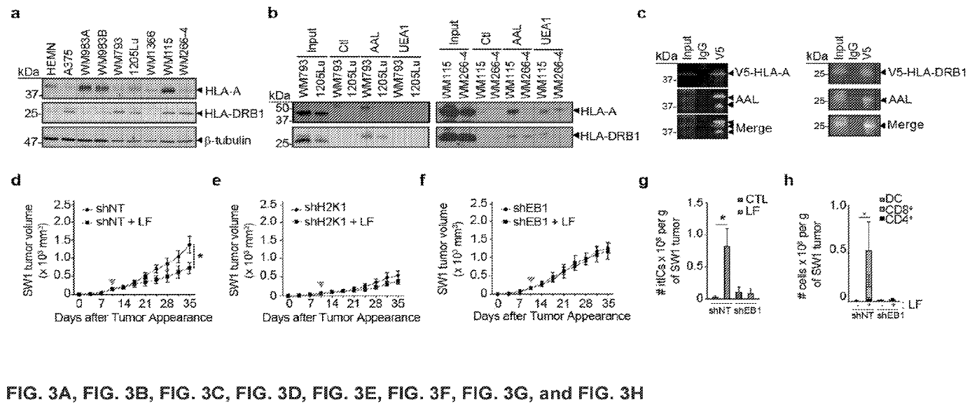

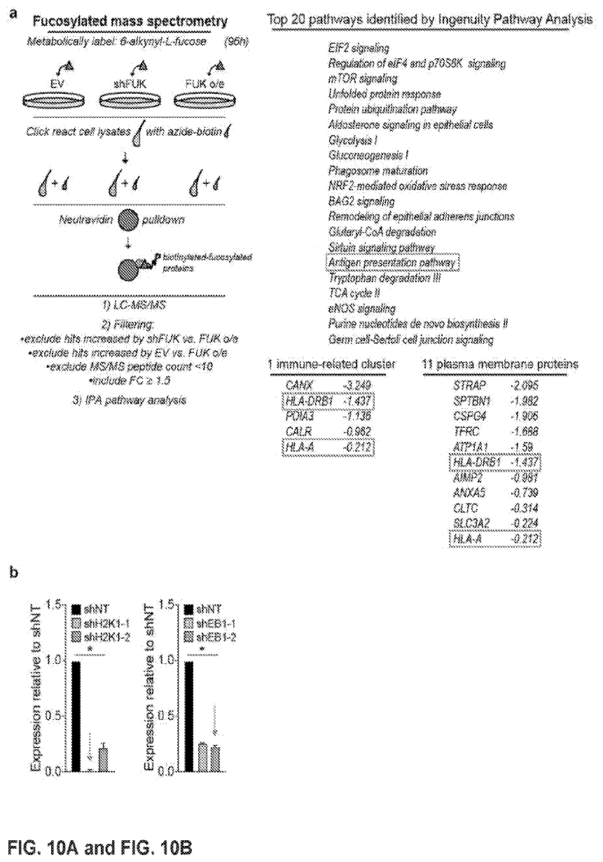

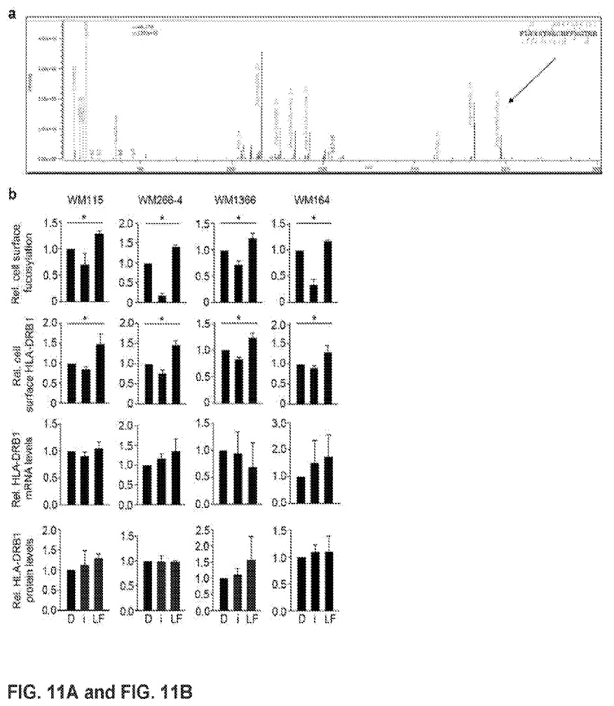

To identify melanoma proteins that contributed to fucosylation-triggered, CD4⁺T cell-mediated melanoma suppression, we subjected fucosylated proteins from human melanoma cells to liquid chromatography mass spectrometric (LC-MS/MS) analysis followed by Ingenuity Pathway Analysis (FIG. 10a, left). These analyses identified “Antigen presentation pathway” as the only immune-related pathway, in which the MHC-I and MHC-II proteins HLA-A and HLA-DRB1, respectively, were identified as the only antigen presentation and plasma membrane proteins with T cell-modulating functions (FIG. 10a, right). We confirmed their expression in human melanocytes and melanoma cells by immunoblot (IB) analysis (FIG. 3a). Further, lectin pulldown (LPD) using Aleuria aurantia (AAL) and Ulex europaeus agglutinin I (UEA1) lectins, which bind to common core and terminal fucosylated glycans, respectively, revealed association of both proteins with AAL (and to a lesser extent, UEA1), indicating N′-linked core glycosylation-fucosylation (FIG. 3b). Finally, immunoprecipitation (IP) and IB analysis of V5-tagged HLA-A or HLA-DRB1 revealed direct recognition of HLA-DRB1 by AAL—indicating that a fraction of total HLA-DRB1, but not HLA-A, is directly fucosylated in melanoma (FIG. 3c).

To determine contributions of HLA-A or HLA-DRB1 to fucosylation-triggered anti-tumor immunity, we knocked down their C3H/HeN mouse orthologs H2K1 or EB1, respectively, in SW1 tumors (FIG. 10b) and assessed growth and TILs in vivo. Whereas L-fuc impaired control tumor growth, H2K1 knockdown suppressed tumor growth regardless of L-fuc (FIG. 3d, 3e), reflecting tumor-protective, immunosuppressive roles of MHC-I proteins. Notably, EB1 knockdown completely abolished L-fuc-triggered tumor suppression and induction of total itICs, including DCs, CD8⁺ and CD4⁺T cell subpopulations (FIG. 3f-3h)-similar to the effects elicited by CD4⁺T cell depletion (FIG. 1l-1o).

Consistent with roles of HLA-DRB1 in CD4⁺T cell activation, our findings demonstrate that HLA-DRB1 is expressed and fucosylated in melanoma and required for L-fuc-triggered CD4⁺T cell-mediated TIL induction and melanoma suppression.

### (4) N48 Fucosylation of HLA-DRB1 Regulates its Cell Surface Localization and is Required for TIL Induction, Anti-Melanoma Immunity, and Melanoma Suppression

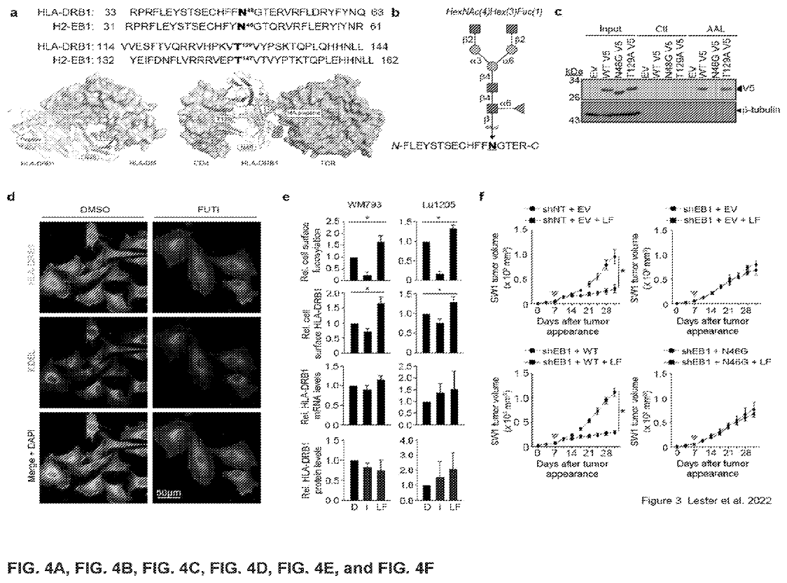

We reasoned that determining how HLA-DRB1 is regulated by fucosylation provides important insight into its crucial role in L-fuc-triggered anti-tumor immunity. Using NetNGlyc and NetOGlyc, we predicted N- and O-linked glycosylation sites at Asn48 (N48) and Thr129 (T129), respectively, which are conserved sites within constant regions of human and mouse HLA-DRB1 (FIG. 4a, upper). Importantly, EB1 exhibits ˜80% sequence homology of HLA-DRB1 and contains the conserved glycosylation-fucosylation site a N46. Modeling of HLA-DRB1 interactions with prominent binding partners HLA-DM or CD4/TCR indicates that fucosylation of neither site affects interaction interfaces or peptide loading/presentation (FIG. 4a, lower).

Nano-LC/MS/MS analysis of HLA-DRB1 immunoprecipitated from WM793 cells identified the fragment FLEYSTSECHFFNGTER as glycosylated-fucosylated at N48 with the predicted glycan HexNAc(4)Hex(3)Fuc(1) (FIG. 4b and FIG. 11a). We mutated N48 or T129 to Gly or Ala, respectively, to abolish and verify fucosylation. Unlike wild-type (WT) or the T129A “glyco-fucomutant” HLA-DRB1, the N48G glyco-fucomutant did not bind to AAL in LPD assays (FIG. 4c), confirming fucosylation at N48 on an N-linked glycan.

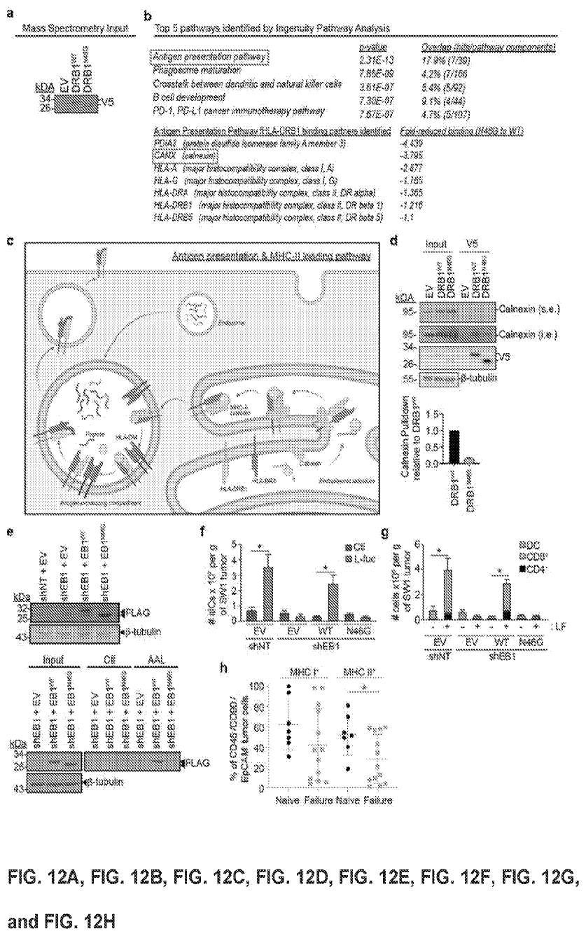

To determine how fucosylation can regulate HLA-DRB1, we assessed its subcellular localization in WM793 cells that were pharmacologically modulated for fucosylation by treatment with 2F-peracteyl-fucose (FUTi, a fucosyltransferase inhibitor) versus vehicle (dimethylsulfoxide, DMSO; control). Cells treated with FUTi exhibited dimmer, more central, endoplasmic reticulum-co-localization of HLA-DRB1 compared with vehicle-treated cells, indicating less accumulation at the cell surface (FIG. 4d). Further, flow cytometry revealed that cell surface fucosylation and HLA-DRB1 both decreased or increased after FUTi or L-fuc treatments, respectively, whereas mRNA and protein levels remained unchanged; thus fucosylation promotes cell surface localization of HLA-DRB1 (FIG. 4e and FIG. 11b). Finally, global proteomic profiling to identify interactors that can mediate fucosylation-regulated cell surface localization of HLA-DRB1 revealed that N48 glycosylation-fucosylation promotes binding to calnexin, which has been reported to mediate maturation and trafficking of MHC-II complexes to the surface (FIG. 12a-12d).

To assess how HLA-DRB1 glycosylation-fucosylation contributes to tumor suppression and TILs, we compared control- or EB1-knocked-down SW1 tumors reconstituted with WT or glyco-fucomutant (N46G) EB1 (confirmation of knockdown-reconstitution and ficosylation by IB and LPD, respectively in FIG. 12e). Abrogation of L-fuc-induced TIL and tumor growth suppression by EB1 knockdown was rescued by reconstitution with only WT but not glyco-fucomutant EB1, demonstrating that glycosylation-fucosylation of EB1/HLA-DRB1 is essential for L-fuc-triggered TIL induction and melanoma suppression (FIG. 4f, FIG. 12f, and FIG. 12g). This is consistent with our finding that loss of glycosylation-fucosylation of HLA-DRB1/EB1 abrogates its cell surface localization and impairs its ability to induce anti-tumor immunity. Thus, despite the other fucosylated proteins identified in melanoma cells (FIG. 10), these data confirm that the N48 glycosylation-fucosylation of HLA-DRB1 is a key regulator of anti-melanoma immunity and tumor suppression. Despite other potential host physiological effects of dietary L-fuc (e.g., microbiome, metabolome, etc.), these data confirm that L-fuc-induced itIC increases and melanoma suppression are critically mediated by melanoma-intrinsic expression and fucosylation of HLA-DRB1, which promotes its cell surface accumulation to trigger CD4⁺T cell-mediated anti-tumor immune responses.

### (5) Oral L-Fucose Augments Anti-PD1-Mediated Melanoma Suppression

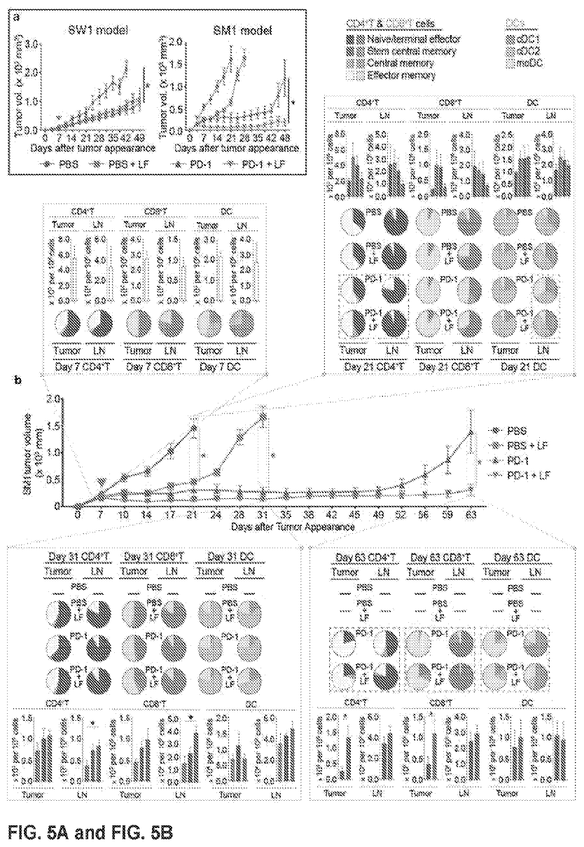

Expression of MHC-JJ reportedly correlates with increased anti-PD1 efficacy. Indeed, patients who failed anti-PD1 exhibited relative >45% reduced cell surface MHC-JI but not MHC-I (FIG. 12h). As anti-PD1 efficacy can be limited by TIL abundance, particularly of CD4⁺T and memory CD4⁺T cells, we tested if the ability to increase CD4⁺T cell-mediated TIL induction and tumor suppression using oral L-fuc can be leveraged to augment anti-PD1 efficacy. In the SW1 model, oral L-fuc suppressed tumors as much as anti-PD1 but did not enhance efficacy of anti-PD1 (˜50-60%; FIG. 5a (left)). In contrast, in the SM1 model, L-fuc was less tumor suppressive than anti-PD1 alone but rather augmented durable suppression in combination with anti-PD1 (FIG. 5a (right)).

To clarify how the L-fuc+anti-PD1 combination enhanced suppression, we characterized immune cell profiles in the tumors and lymph nodes of SM1 tumor-bearing mice over a timecourse of treatment with L-fuc±anti-PD1. Administration of L-fuc (i) alone increased intratumoral CD4⁺T central and effector memory cells, an effect that was increased when combined with anti-PD1 (FIG. 5b (blue dashed boxes)), and (ii), initially expanded intratumoral cDC2s, followed by later expansion of cDC2s and moDCs in the lymph nodes when combined with anti-PD1 (FIG. 5b (orange dashed boxes)). In addition to expanding the absolute numbers of intratumoral CD4⁺ and CD8⁺ T cells at endpoint (Day 63), combination L-fuc+anti-PD1 increased the relative percentage of intratumoral CD8⁺ T central memory cells (FIG. 5b (green dashed box)). Thus, L-fuc can suppress some melanomas as effectively as anti-PD1, whereas in others, it can enhance efficacy, which is associated with increased intratumoral CD4⁺T central and effector memory subpopulations and lymph node cDC2 and moDC populations, consistent with the effects of L-fuc observed in FIG. 2.

### (6) Melanoma Fucosylation and Fucosylated HLA-DRB1 as Biomarkers of Anti-PD1 Response

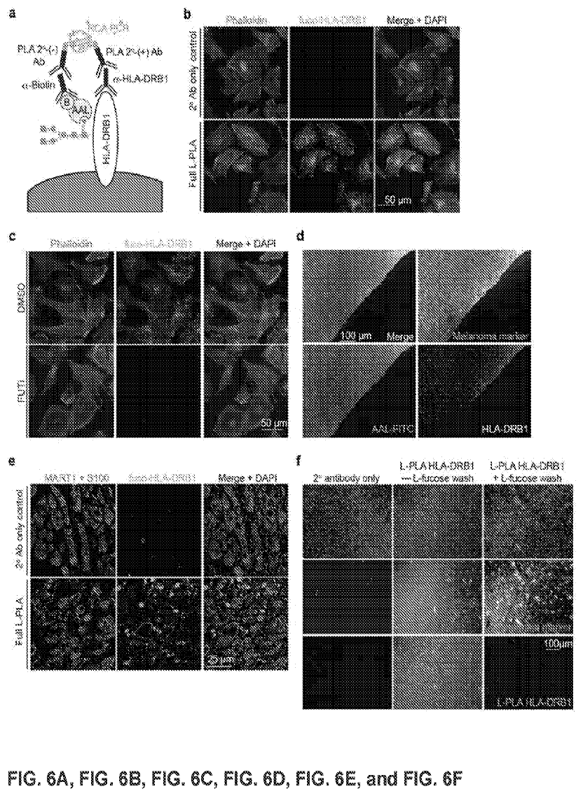

Given the potent enhancement of anti-PD1 efficacy by oral L-fuc in mice, we assessed if tumor fucosylation or total/fucosylated HLA-DRB1 can correlate at all with responsiveness to anti-PD1 in human patient biopsies, as the identification of correlations can support their subsequent development in into predictive biomarkers for anti-PD1 responsiveness. To this end, we devised a new technique: we modified conventional proximity ligation assay (PLA) to facilitate immunofluorescent visualization of fucosylated HLA-DRB1 by implementing anti-HLA-DRB1 antibody together with biotinylated AAL, which has been successfully used to stain tissues specifically for core-fucosylated glycan⁵⁷ (FIG. 6a). This technique, lectin-mediated PLA (L-PLA), revealed cytoplasmic/membranous localization of endogenous fucosylated HLA-DRB1 in melanoma cells (FIG. 6b) that is lost upon FUTi treatment (FIG. 6c), confirming L-fuc-stimulated cell surface localization of HLA-DRB1 (FIG. 4d, 4e and FIG. 11b). The cytoplasmic/“vesicular-appearing” staining is consistent with HLA-DRB1 that was fucosylated in the ER/Golgi and is en route to the surface via the secretory pathway. In applying this technique further on FFPE melanoma tissue specimens, we observed similar staining patterns for fucosylated HLA-DRB1 (FIG. 6d, 6e), which were completely abolished by L-fuc washing of the tissue, confirming specificity for fucosylated HLA-DRB1 (FIG. 6f).

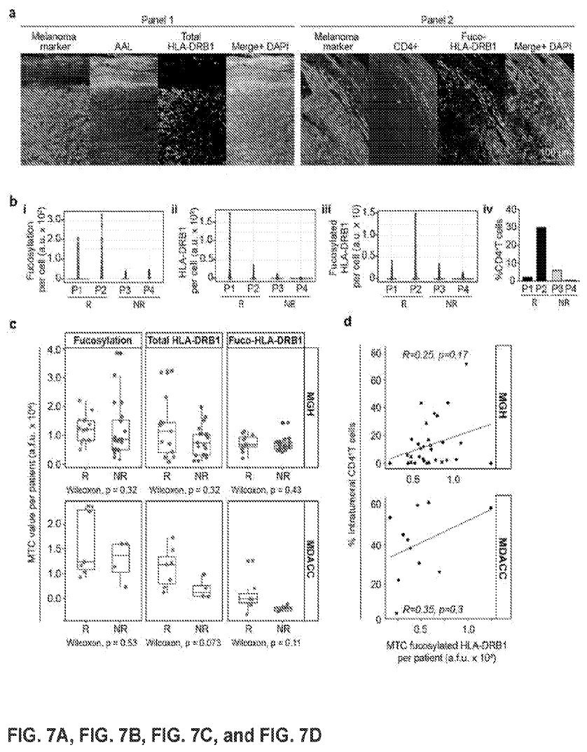

To assess correlations of (i) tumor-specific fucosylation and total/fucosylated HLA-DRB1 of individual tumor cells, and (ii) intratumoral numbers CD4⁺T cells with responder D status to single-agent anti-PD1, we implemented L-PLA on primary melanoma biopsies from 2 distinct responder and 2 non-responder patients followed by single-cell segmented signal quantitation (FIG. 7a, 7b). Tumors of responders clearly contained tumor cell populations with high levels of fucosylation and total HLA-DRB1 versus non-responders (FIG. 7b.i,b.ii). Although the tumor of only 1 of 2 responders contained melanoma cells with increased levels of fucosylated HLA-DRB1 compared with the non-responders (FIG. 7b.iii), this trend mirrored that of intratumoral CD4⁺T cell counts (FIG. 7b.iv), consistent with the role for fucosylated HLA-DRB1 in CD4⁺T cell-mediated tumor suppression.

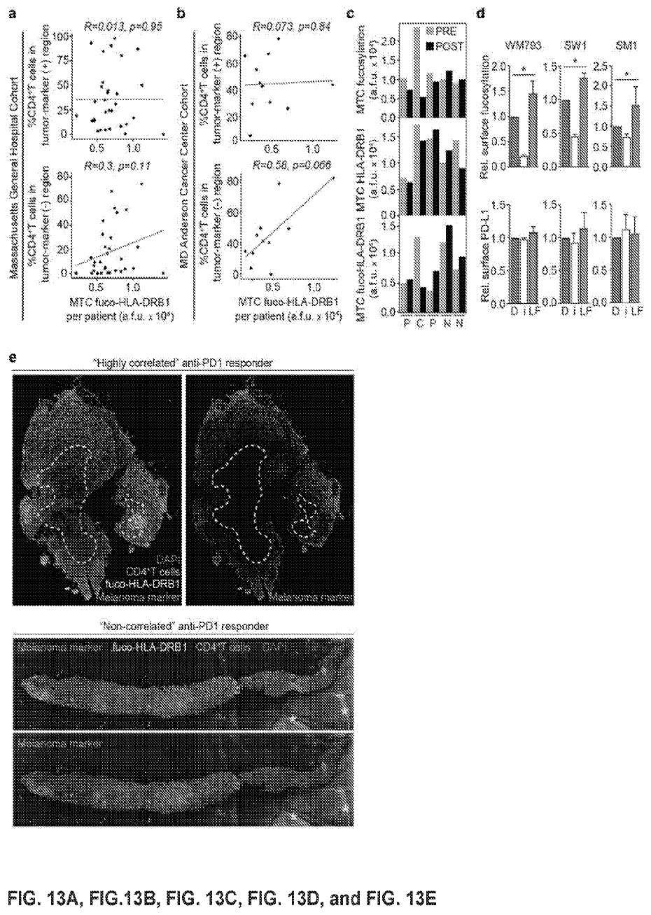

We assessed associations between tumor fucosylation, total/fucosylated HLA-DRB1, CD4⁺T cells and responder status in expanded cohorts of anti-PD1-treated melanoma patients. Levels of tumor fucosylation and total and fucosylated HLA-DRB1 in tumor cells were generally higher in anti-PD1 responders compared with non-responders from Massachusetts General Hospital (n=31; FIG. 7c, upper) and MD Anderson Cancer Center (n=11; FIG. 7c, lower). Total tumor fucosylated HLA-DRB1 exhibited weak or no association with tumoral CD4⁺T cell (FIG. 7d, upper & lower), although the association was modestly increased when restricted to CD4⁺T cells localized at the periphery of the tumors (FIGS. 13a and 13b; absolute CD4⁺T numbers in Table 3). The lack of significant correlation can be attributed to the dynamic relationship between fucosylated HLA-DRB1 and CD4⁺T infiltration that is further weakened by suboptimal inclusion criteria/patient stratification. Comparison of these markers in 5 patient-matched pre- and post-anti-PD1 tumors revealed no significant correlation in total HLA-DRB1 levels. However, prior to treatment, tumor cell fucosylation was significantly higher in the complete responder versus partial and non-responders; this dropped to the equivalently lower levels of the other patients after treatment. With the exception of 1 non-responder, the complete responder also exhibited significantly increased fucosylated HLA-DRB1 in tumor cells prior to treatment (FIG. 13c).

TABLE 3

| | % CD4 of | % CD4 of | % CD4 of total | absolute # | | | |
|---|---|---|---|---|---|---|---|
| | total cells in | total cells | cells in tumor | CD4 cells in | absolute # | absolute # | Patient |
| Moffitt | tumor | within | periphery + | tumor | CD4 cells in | CD4 cells in | responder |
| Patient # | periphery | tumor area | tumor areas | periphery | tumor area | total | status |
| 1 | 1.65 | 2.59 | 2.42 | 2085.00 | 14427.00 | 16512.00 | Y |
| 2 | 10.68 | 30.36 | 29.76 | 2519.00 | 225655.00 | 228174.00 | Y |
| 3 | 5.33 | 6.32 | 6.17 | 929.00 | 6046.00 | 6975.00 | N |
| 4 | 0.00 | 0.93 | 0.93 | 0.00 | 125.00 | 125.00 | N |

| | % CD4 of | % CD4 of | % CD4 of total | absolute # | | | |
|---|---|---|---|---|---|---|---|
| Massachusetts | total cells in | total cells | cells in tumor | CD4 in | absolute # | absolute # | Patient |
| General Hospital | tumor | within | periphery + | tumor | CD4 in | CD4 in | responder |
| Patient # | periphery | tumor area | tumor areas | periphery | tumor area | total | status |
| 1 | 6.48 | 22.70 | 22.65 | 42.00 | 57491.00 | 57533.00 | N |
| 2 | 45.80 | 86.92 | 84.07 | 1295.00 | 33474.00 | 34769.00 | Y |
| 3 | 87.02 | 15.23 | 15.86 | 8154.00 | 160252.00 | 168406.00 | N |
| 4 | 5.61 | 8.01 | 7.37 | 62.00 | 240.00 | 302.00 | Y |
| 5 | 12.29 | 33.20 | 23.78 | 1061.00 | 3490.00 | 4551.00 | N |
| 6 | 6.72 | 5.46 | 5.92 | 9128.00 | 12865.00 | 21993.00 | Y |
| 7 | 22.18 | 44.67 | 44.45 | 645.00 | 130928.00 | 131573.00 | N |
| 8 | 16.72 | 18.83 | 17.29 | 2669.00 | 1125.00 | 3794.00 | Y |
| 9 | 0.00 | 0.00 | 0.00 | 0.00 | 0.00 | 0.00 | N |
| 10 | 27.97 | 80.35 | 50.93 | 2112.00 | 4738.00 | 6850.00 | N |
| 11 | 11.45 | 30.63 | 14.12 | 1117.00 | 480.00 | 1597.00 | N |
| 12 | 21.09 | 41.25 | 40.94 | 58.00 | 7218.00 | 7276.00 | N |
| 13 | 47.56 | 93.19 | 78.13 | 4804.00 | 19092.00 | 23896.00 | N |
| 14 | 43.65 | 90.26 | 86.89 | 12344.00 | 327694.00 | 340038.00 | N |
| 15 | 2.63 | 48.32 | 47.46 | 3.00 | 2869.00 | 2872.00 | N |
| 16 | 18.04 | 52.94 | 50.11 | 239.00 | 7929.00 | 8168.00 | N |
| 17 | 0.59 | 0.47 | 0.48 | 6.00 | 93.00 | 99.00 | N |
| 18 | 24.40 | 84.55 | 27.95 | 8487.00 | 1849.00 | 10336.00 | N |
| 19 | 3.48 | 4.38 | 4.17 | 59.00 | 238.00 | 297.00 | N |
| 20 | 92.63 | 97.81 | 96.49 | 16805.00 | 52091.00 | 68896.00 | N |
| 21 | 10.77 | 10.05 | 10.31 | 4518.00 | 7505.00 | 12023.00 | Y |
| 22 | 73.99 | 49.95 | 61.24 | 21319.00 | 16242.00 | 37561.00 | Y |
| 23 | 0.61 | 4.39 | 4.18 | 20.00 | 2390.00 | 2410.00 | Y |
| 24 | 93.75 | 3.41 | 3.53 | 270.00 | 7084.00 | 734.00 | Y |
| 25 | 53.81 | 13.70 | 43.15 | 33884.00 | 3123.00 | 37007.00 | Y |
| 26 | 70.63 | 35.09 | 40.71 | 14032.00 | 37108.00 | 51140.00 | N |
| 27 | 51.66 | 16.61 | 29.95 | 12627.00 | 6610.00 | 19237.00 | Y |
| 28 | 89.46 | 13.98 | 15.28 | 611.00 | 5456.00 | 6067.00 | N |
| 29 | 86.36 | 23.94 | 23.95 | 19.00 | 46456.00 | 46475.00 | N |
| 30 | 42.14 | 42.45 | 42.35 | 4936.00 | 9944.00 | 14880.00 | Y |
| 31 | 74.84 | 24.33 | 30.30 | 14558.00 | 35355.00 | 49913.00 | Y |

| | % CD4 of | % CD4 of | % CD4 of total | absolute # | | | | |
|---|---|---|---|---|---|---|---|---|
| MD Anderson | total cells in | total cells | cells in tumor | CD4 in | | | Patient | |
| Cancer Center | tumor | within | periphery + | tumor | absolute # CD4 | absolute # | responder | Specific |
| Patient # | periphery | tumor area | tumor areas | periphery | in tumor area | CD4 in total | status | Response |
| 1-pre | 6.53 | 6.32 | 6.49 | 5495.00 | 1353.00 | 6848.00 | N | NR |
| 1-post | 52.04 | 34.55 | 38.91 | 39725.00 | 79485.00 | 119210.00 | N | |
| 2 | 1.50 | 0.19 | 0.99 | 2803.00 | 221.00 | 3024.00 | Y | |
| 3 pre | 12.11 | 23.90 | 15.53 | 127113.00 | 102401.00 | 229514.00 | N | NR |
| 3-post | 52.03 | 54.86 | 52.98 | 83758.00 | 45164.00 | 128922.00 | N | |
| 4 | 53.93 | 33.42 | 35.71 | 29474.00 | 144672.00 | 174146.00 | Y | |
| 5 | 47.05 | 20.60 | 42.75 | 109930.00 | 9327.00 | 119257.00 | N | |
| 6 | 10.50 | 14.38 | 12.51 | 2250.00 | 3314.00 | 5564.00 | N | |
| 7 | 30.81 | 31.59 | 31.23 | 26472.00 | 30790.00 | 57262.00 | N | |
| 8 | 9.00 | 25.36 | 11.31 | 65408.00 | 30320.00 | 95728.00 | N | |
| 9 | 8.62 | 19.88 | 18.71 | 6409.00 | 127163.00 | 133572.00 | N | |
| 11-pre | 29.92 | 45.18 | 42.31 | 63950.00 | 416310.00 | 480260.00 | Y | PR |
| 11-post | 5.11 | 7.71 | 6.90 | 26282.00 | 87359.00 | 113641.00 | Y | |
| 12 | 13.69 | 6.62 | 9.10 | 12685.00 | 11309.00 | 23994.00 | N | |
| 13 | 52.74 | 73.33 | 71.72 | 2222.00 | 36390.00 | 38612.00 | Y | |
| 14 | 29.62 | 1.57 | 6.20 | 135544.00 | 36368.00 | 171912.00 | Y | |
| 15 | 84.84 | 69.77 | 75.36 | 251809.00 | 351230.00 | 603039.00 | Y | |
| 16 | 25.00 | 24.05 | 24.40 | 2163.00 | 3520.00 | 5683.00 | N | |
| 17-pre | 61.96 | 43.49 | 58.43 | 3123.00 | 518.00 | 3641.00 | Y | CR |
| 17-post | 23.13 | 39.20 | 37.49 | 33706.00 | 481246.00 | 514952.00 | Y | |
| 18 | 39.92 | 6.52 | 21.75 | 389442.00 | 75875.00 | 465317.00 | Y | |
| 19 | 73.91 | 95.25 | 74.86 | 60561.00 | 3648.00 | 64209.00 | Y | |
| 20-pre | 0.00 | 30.74 | 30.67 | 0.00 | 2450.00 | 2450.00 | N | PR |
| 20-post | 71.93 | 47.03 | 68.90 | 1951316.00 | 176634.00 | 2127950.00 | N | |
| 21 | 20.45 | 66.79 | 59.69 | 29.38 | 806156.00 | 850835.00 | Y | |
| 22 | 19.76 | 79.97 | 78.50 | 14.88 | 5330.00 | 5363.00 | Y | |
| 23 | 21.81 | 39.63 | 34.13 | 25.40 | 433810.00 | 540218.00 | N | |
| 24 | 39.78 | 33.57 | 38.01 | 20.45 | 3875.00 | 15440.00 | Y | |
| 25 | 31.98 | 67.55 | 45.71 | 4.04 | 17722.00 | 31073.00 | N | |
| 26 | 31.48 | 31.07 | 31.26 | 31.48 | 328402.00 | 605492.00 | Y | |

### b) Discussion

For the first time, we report the administration of a dietary sugar as a way to increase TILs and enhance efficacy of the immune checkpoint blockade agent anti-PD1. Further, these studies reveal new insights into the post-translational regulation and immunological roles of melanoma cell-expressed MHC-II proteins, further highlighting their relationship with TILs. Specifically, fucosylation regulates the cell surface abundance of HLA-DRB1, which triggers robust CD4⁺T cell-mediated TIL induction and melanoma suppression. It is important to acknowledge that our reliance on AAL lectin predominantly focuses our study on α1,6-fucosylated proteins. Although this does not diminish the crucial role that α1,6-fucosylated HLA-DRB1—which was identified as fucosylated via lectin-agnostic click chemistry mass spectrometric screening—plays in L-fuc-triggered anti-tumor immune responses, other fucosylation linkages can contribute to aspects of the anti-tumor immunity. Nonetheless, the ability to leverage this mechanism using oral L-fuc can help to enhance other immunotherapeutic modalities (i.e., other checkpoint inhibitors or adoptive cell transfer therapies). Notably, as a non-toxic dietary sugar with a past safety precedent as an experimental therapy for children with Leukocyte Adhesion Deficiency II, L-fuc appears to be a safe and tolerable therapeutic agent.

The consistent trends that we observed in higher tumor fucosylation and fucosylated HLA-DRB1 across anti-PD1 responders vs. non-responders between the 3 independent cancer center cohorts support their utility as biomarkers of anti-PD1 responsiveness. In terms of biological variables, how T cell biology is regulated by fucosylation, for example, has heretofore been unclear. Reported divergent effects of fucosylation on T cell activation vs. exhaustion (i.e., via regulation of Programmed Death Ligand 1 (PD-L1) expression) point to FUT-specific expression and roles that remain to be elucidated. L-fucose does not alter cell surface levels of PD-L1 in human or mouse melanoma cells (FIG. 13d), indicating that the discrepant tumor suppression by single-agent vs. combination L-fuc+anti-PD1 in our SW1 and SM1 mouse models (FIG. 5a) is attributed to determinants beyond the PD1:PD-L1 axis. Indeed, our global fucosylated and phosphoproteomic analyses revealed that fucosylation in CD4⁺T cells impacts Integrin β5, PKA, and actin signaling (FIG. 9), and that this is associated with increased intratumoral T cell presence and memory phenotypes in our models (FIGS. 2 and 5)—consistent with reports that those functions are regulated by those pathways in T cell biology. That L-fuc can increase CD4⁺T central memory cells also partially explains how it can augment anti-PD1 efficacy, which is associated with the presence of these cells. How L-fuc can regulate these signaling pathways and enrich for CD4⁺T memory subsets within the tumor microenvironment, and further, how the intratumoral increases in DC subtypes induced by L-fuc (FIGS. 2 and 5) can contribute to changes in CD4⁺T cell biology and activity, other aspects of anti-tumor immune responses, and tumor suppression in this context are unclear and warrant further lines of study. Further In addition, sex can be a determinant, as melanoma fucosylation levels are lower but correlate more strongly with intratumoral CD3⁺ T cells in male vs. female patients (FIG. 1j, 1k). Reduced melanoma fucosylation, which is expected to lower TILs, can explain increased lethality in male patients (American Cancer Society Facts & Figures, 2021).

In conclusion, fucosylation of HLA-DRB1 is a key regulator of TIL abundance in melanomas, and this mechanism, together with fucosylation-regulated CD4⁺T cell biology, can be therapeutically exploited using oral L-fuc. Elucidation of the mechanistic determinants is expected to advance our understanding of the immunobiology of melanoma and other cancers and to inform efforts at implementing fucosylation/fucosylated HLA-DRB1 as biomarkers and of L-fuc as a therapeutic agent.

### c) Methods

### (1) General Cell Culture

NHEM (normal adult epidermal melanocytes) were grown in Lonza MGM-4 growth media; prior to harvest for IB analysis, the cells were switched to the same media as the other cells overnight. WM793, 1205Lu, A375, WM1366, WM164, and SW1 melanoma cells were obtained from the Ronai laboratory (Sanford-Burnham Prebys Medical Discovery Institute (La Jolla, CA), WM983A/B cells were purchased from Rockland Immunochemicals (Limerick, PA). WM115 and WM266-4 cells were purchased from ATCC (Manassas, VA). SM1 (Gift from the Smalley Laboratory at Moffitt), were cultured in Dulbecco's Modified Eagle Medium containing 10% fetal bovine serum (FBS), 1 g/mL glucose, 4 mM L-glutamine in 37° C. in 5% CO₂. Cell lines were transfected using Lipofectamine 2000 (Invitrogen, Waltham, MA). Primary CD4⁺T cells were harvested using the EasySep (StemCell Technologies) Human CD4⁺ negative selection isolation kit (#17952) according to manufacturer's protocols.

### (2) Antibodies

The following antibodies were used as indicated: mouse anti-V5 (0.2 μg/mL Millipore Sigma (St. Louis, MO)), mouse anti-V5 gel (V5-10, Millipore Sigma (St. Louis, MO)), mouse anti-human HLA-DRB1 (0.2 μg/mL, IF, ab215835, Abcam (Cambridge, UK)), rabbit anti-human HLA-DRB1 (0.2 μg/mL WB, ab92371, Abcam (Cambridge, UK)), 0-tubulin (0.3 μg/mL, E7, developed by M. McCutcheon and S. Carroll and obtained from Developmental Studies Hybridoma Bank (University of Iowa, Iowa City, IA)), goat anti-biotin (0.1 μg/mL Vector Labs (Burlingame, CA)), biotinylated AAL (0.4 μg/mL Vector Labs, Burlingame, CA), fluorescein-conjugated AAL (0.4 μg/mL Vector Laboratories, Burlingame, CA), Agarose UEA1 and AAL (Vector Laboratories, (Burlingame, CA)), anti-mouse CD4 (20 mg/kg, for immunodepletion, GK1.5, Bioxcell (West Lebanon, NH)), anti-mouse CD8 (20 mg/kg, for immunodepletion, 2.43, Bioxcell (West Lebanon, NH)), goat anti-mouse IgGκ horseradish peroxidase (HRP) (0.04 μg/mL, Santa Cruz Biotechnology (Dallas, TX)), mouse anti-rabbit HRP (0.04 μg/mL, Santa Cruz Biotechnology (Dallas, TX)), goat anti-rabbit AlexaFluor 488 (0.04 μg/mL, ThermoFisher Scientific (Waltham, MA))), donkey anti-mouse AlexaFluor 594 (0.05 μg/mL, ThermoFisher Scientific (Waltham, MA)), AlexaFluor 594 donkey anti-rabbit (0.05 μg/mL, ThermoFisher Scientific (Waltham, MA)), rabbit anti-Mart1 (0.2 μg/mL, Millipore Sigma (St. Louis, MO), rabbit anti-S100 (0.2 μg/mL, Agilent Technologies (Santa Clara, CA)), APC anti-mouse CD3 (0.5 μg/mL, Biolegend (San Jose, CA)), Pacific Blue anti-mouse CD4 (0.5 μg/mL, BD Biosciences (San Jose, CA)), BV785 anti-mouse CD8 (0.5 μg/mL, BD Biosciences (San Jose, CA)), FITC anti-mouse F4/80 (0.5 μg/mL, BD Biosciences (San Jose, CA)), APC anti-mouse GR-1 (0.5 μg/mL, BD Biosciences (San Jose, CA)), PeCy7 anti-mouse CD11c (0.5 μg/mL, BD Biosciences (San Jose, CA)), PE anti-mouse NK1.1 (0.5 μg/mL, BD Biosciences (San Jose, CA)), PE anti-mouse DX5 (0.5 μg/mL, BD Biosciences (San Jose, CA)), PerCP-Cy5.5 anti-mouse CD11b (0.5 μg/mL, BD Biosciences (San Jose, CA)), rabbit anti-human PD-L1 (clone #NBP1-76769; Noveus Biologicals, Centennial, CO), PE rat anti-mouse PD-L1 (clone #10F.9G2; (Biolegend, (San Diego, CA)), and phalloidin Alexafluor 488 (0.2 μg/mL, ThermoFisher Scientific (Waltham, MA)), mouse anti-FLAG (0.2 μg/mL, clone M2, Millipore Sigma (St. Louis, MO)), rabbit anti-HLA-A (0.2 μg/mL, Proteintech (Rosemont, IL)), normal mouse IgG (Santa Cruz Biotechnology (Dallas, TX)), rabbit anti-KDEL (0.1 μg/mL, ThermoFisher Scientific (Waltham, MA)), mouse anti-PD1 (for in vivo studies, 20 mg/kg, clone #RMP1-14 Bioxcell (West Lebanon, NH)), donkey anti-goat plus PLA secondary antibody (Millipore Sigma (St. Louis, MO)), donkey anti-mouse plus PLA secondary antibody (Millipore Sigma (St. Louis, MO)), rat anti-mouse CD8 antibody (0.2 μg/mL, ThermoFisher Scientific (Waltham, MA)), AlexaFluor 594 goat anti-rat secondary antibody (0.05 μg/mL, ThermoFisher Scientific (Waltham, MA)), anti-CD3 (0.2 μg/mL, Clone PS1, Santa Cruz Biotechnology (Dallas, TX), PE anti-pan-MHC-I (HLA-A,B,C) (BD Pharmingen (San Jose, CA), FITC anti-pan-MHC-II (HLA-DP, DQ, DQ)(BD Pharmingen (San Jose, CA), PerPCy5.5 anti-CD45 (Invitrogen (Waltham, MA)), APC anti-CD90 (Biolegend (San Diego, CA)), and BV421 anti EpCAM (Biolegend (San Diego, CA)).

### (3) Cloning and Mutagenesis

Mouse fucokinase (mFuk) was cloned using cDNA from SW1 cells into pLenti-C-Myc-DDK-IRES-Puro expression vector (Origene Technologies (Rockville, MD)) into BAMHI and NHEI restriction sites. Mouse EB1 constructs was cloned using cDNA from SW1 cells into pLenti-C-Myc-DDK-IRES-Puro expression vector (Origene Technologies (Rockville, MD)) into ASCI and XHOI restriction sites. pLKO Non-targeting shRNA (shNT), pLKO shK1-1, pLKO shK1-2, pLKO shEB1-1, and pLKO shEB1-2 were obtained from Millapore Sigma (St. Louis). pLX304::EV was obtained from Origene Technologies (Rockville, MD). pLX304::HLA-A and pLX304::HLA-DRB1 constructs were obtained from DNAasu (PMID:21706014). HLA-DRB1 N48G and T129A as well as EB1 N46G mutants were generated using QuikChange II XL site-directed mutagenesis kit according to the manufacturer's protocol (Agilent Technologies (Santa Clara, CA)).

### (4) Proteomic Mass Spectrometric Profiling of Fucosylated Proteins

WM793 cells stably transduced with pLenti-GFP empty vector (EV), pLenti-FUK-GFP, or shFUK were grown in triplicate to ˜30-40% confluence in (3×15 cm3 plates each). The cells were further cultured in the presence of 50 μM L-fucose-alkyne for ˜72 h to ˜80% confluence. The cells were lysed in 1.5% N-dodecyl-beta-D-maltoside/20 mM HEPES pH 7.4/protease and phosphatase inhibitors. Lysates were sonicated and cleared by centrifugation at full speed for 5 min at 4 C. Lysates were acetone precipitated overnight. The pelleted proteins were resuspended and subjected to click-chemistry labeling with biotin-azide using the Click-It kit per manufacturer's protocol (Invitrogen). For negative control, pLenti-GFP-EV cells were not labeled with L-fucose-alkyne but were lysed, pelleted, and click-reacted with biotin-azide. All biotin-azide (biotinylated-fucosylated) samples were pulled down using neutravidin beads that were pre-blocked with 2% IgG-free BSA. Samples were submitted to the Sanford-Burnham Prebys proteomics core facility for on-bead digest; supernatants from on-bead digest were analyzed by LC/MS/MS. Hits that were increased by >1.5 fold in pLenti-FUK-GFP-expressing cells and unchanged or decreased in pLenti-EV-GFP-expressing cells or decreased in pLenti-shFUK-expressing cells. Hits were subjected to Ingenuity Pathway Analysis (Qiagen).

### (5) Lectin Pulldown

Control beads and AAL or UEA1 lectin-conjugated agarose beads were pre-blocked for 2 h in blocking buffer (2% IgG-Free BSA (Jackson ImmunoResearch Laboratories (Westgrove, PA)) on a rotator at 4° C. Cells were lysed on ice in 1% Triton-X100 lysis buffer (1% Triton-X100, 20 mM Tris-HCl, pH 7.4, 150 mM NaCl in ddH2O+protease and phosphatase inhibitors (ThermoFisher Scientific (Waltham, MA)), briefly sonicated, pelleted, and the resulting lysates were normalized in protein concentration to the sample with the lowest concentration and diluted to a final 0.25% Triton-X-100 with dilution buffer (0% Triton X-100, 20 mM Tris-HCl, pH 7.4, 150 mM NaCl in ddH₂O+protease and phosphatase inhibitors (ThermoFisher Scientific (Waltham, MA)), and incubated with 15 μl of pre-blocked beads (beads were spun out of block and resuspended in dilution buffer) and rotated overnight at 4° C. Next, the beads were washed twice with dilution buffer and subjected to (12%) SDS-PAGE and IB analysis using the indicated antibodies.

### (6) Mass Spectrometric Analysis of Glycosylation on HLA-DRB1

Stained bands of approximately lug of exogenously expressed V5-HLA-DRB1 purified from WM793 cells were cut into 1-mm³ pieces and reduced and alkylated using 20 mM TCEP (tris(2-carboxyehtyl)phosphine) and iodoacetamide in 50 mM Tris-HCl. The gel pieces were washed in a 20 mM ammonium phosphate solution with 50% methanol overnight at 4° C. The following day, the gel pieces were dehydrated for 30 minutes with 100% acetonitrile. After gel pieces were completely dry, trypsin protease solution was added to the samples (300 ng trypsin). Samples were digested for 4 hours at 37° C. The digests were applied to a C-18 Zip-Tip and eluted with 50% methanol and 0.1% formic acid. Five microliters of the elution were diluted in 0.1% formic acid and then injected into a Q-Exactive Orbitrap mass spectrometer (ThermoFisher Scientific, (Waltham, MA)) equipped with an Easy nano-LC HPLC system with reverse-phase column (ThermoFisher Scientific, (Waltham, MA)). A binary gradient solvent system consisting of 0.1% formic acid in water (solvent A) an 90% acetonitrile and 0.1% formic acid in water (solvent B) was used to separate peptides. Raw data files were analyzed using both Proteome Discoverer v2.1 (ThermoFisher Scientific, (Waltham, MA)) with Byonic (Protein Metrics) as a module and Byonic standalone v2.10.5. All extracted ion chromatograms (EICs) were generated using Xcalibur Qual Browser v4.0 (ThermoFisher Scientific, (Waltham, MA)). UniProt sequence Q5Y7D1_Human was used as the reference sequence for peptide analysis.

### (7) Phosphoproteomics Mass Spectrometric Profiling of CD4⁺T Cells

CD4⁺T cells cultured and treated as indicated in the main text were harvested and lysed in standard RIPA buffer+protease and phosphatase inhibitors. Protein concentration was estimated by BCA assay (Bio-Rad) and 1 mg lysates were subjected to trypsin digestion. Briefly, lysates were reduced with 4.5 mM dithiothreitol (DTT) for 30 min at 60° C., alkylated with 10 mM iodoacetamide (IAA) at room temperature in the dark for 20 minutes, and digested overnight at 37° C. with 1:20 enzyme-to-protein ratio of trypsin (Worthington). The resulting peptide solution was de-salted using reversed-phase Sep-Pak C₁₈ cartridge (Waters) and lyophilized for 48 hours.

### (a) Phosphopeptide Enrichment by IMAC Fe-NTA Magnetic Beads:

The lyophilized peptides were enriched for global phosphopeptides (pSTY) using IMAC Fe-NTA magnetic beads (Cell Signaling Technology, #20432). Enrichment were carried out on a KingFisher™ Flex Purification System (Thermo Fisher, #24074441). The enriched peptides were concentrated in a SpeedVac and suspended in 15 μL loading buffer (5% ACN and 0.1% TFA) prior to auto sampling. Samples were then subjected to LC-MS/MS as described below

### (8) Fuco-Proteomic Mass Spectrometric Profiling of CD4⁺T Cells

CD4⁺T cells cultured and treated as indicated in the main text were harvested, lysed in standard RIPA buffer+protease and phosphatase inhibitors, and subjected to lectin pulldown using control or AAL beads as described above. The beads were washed with PBS and subjected to on-bead trypsin digestion. Proteins bound to beads were denatured with 30 mM ammonium bicarbonate at 95° C. for 5 minutes. Samples were reduced with 4.5 mM dithiothreitol (DTT) for 30 min at 60° C., alkylated with 10 mM iodoacetamide (IAA) at room temperature in the dark for 20 minutes, and digested overnight at 37° C. with 1:20 enzyme-to-protein ratio of trypsin (Worthington). The resulting peptide solution was acidified with a final concentration of 1% TFA. Samples were centrifuged at high speed and the supernatants were subjected to Ziptip purification (Millipore Ziptips, #Z720070). The eluted peptides were concentrated in a SpeedVac and suspended in 15 μL loading buffer (5% ACN and 0.1% TFA) prior to auto sampling. Samples were then subjected to LC-MS/MS as described below

(9) Mass Spectrometric Identification of WT Vs. Glycofucomutant HLA-DRB1 Interactors

V5-tagged WT or N48G glycofucomutant HLA-DRB1-expressing WM793 cells were lysed and subjected to V5 bead pulldown. Five percent of pulled down protein was immunblotted to ensure for equal sample submission for LC-MS/MS (Extended Data FIG. 5a).

Samples were then subjected to LC-MS/MS as described below.

### (10) Liquid Chromatography-MS/MS

On-bead digestion was performed with trypsin and tryptic peptides were then analyzed using a nanoflow ultra-high-performance liquid chromatograph (RSLC, Dionex, Sunnyvale, CA) coupled to an electrospray orbitrap mass spectrometer (Q-Exactive Plus, Thermo, San Jose, CA) for tandem mass spectrometry peptide sequencing. The peptide mixtures were loaded onto a pre-column (2 cm×100 μm ID packed with C18 reversed-phase resin, 5 μm, 100 Å) and washed for 5 minutes with aqueous 2% acetonitrile and 0.1% formic acid. The trapped peptides were eluted and separated on a 75 μm ID×50 cm, 2 μm, 100 Å, C18 analytical column (Dionex, Sunnyvale, CA) using a 90-minute program at a flow rate of 300 nL/min of 2% to 3% solvent B over 5 minutes, 3 to 30% solvent B over 27 minutes, then 30% to 38.5% solvent B over 5 minutes, 38.5% to 90% solvent B over 3 minutes, then held at 90% for 3 minutes, followed by 90% to 2% solvent B in 1 minute and re-equilibrated for 18 minutes. Solvent A was composed of 98% ddH₂O and 2% acetonitrile containing 0.1% FA. Solvent B was 90% acetonitrile and 10% ddH₂O containing 0.1% FA. MS resolution was set at 70,000 and MS/MS resolution was set at 17,500 with max IT of 50 ms. The top sixteen tandem mass spectra were collected using data-dependent acquisition (DDA) following each survey scan. MS and MS/MS scans were performed in an Orbitrap for accurate mass measurement using 60 second exclusion for previously sampled peptide peaks. MaxQuant software (version 1.6.2.10) was used to identify and quantify the proteins for the DDA runs.

### (11) PyMOL Structural Modeling

In FIG. 4a, structural modeling was performed using PyMOL (Molecular Graphics System, Version 2.0 Schrödinger, LLC) of the HLA-DRB1:HLA-DM complex (PDB ID, 4FQX); HLA-DRB1 (yellow) and DM (gray). For the CD4:HLA-DRB1:TCR complex, the model was reconstituted by superimposing the DRB1 beta chains from CD4:HLA-DR1 complex (PDB ID, 3S5L) and TCR:HLA-DR1 complex (PDB ID, 6CQR) using PyMOL. RMSD between the 163 backbone atoms is 0.497. The glycosylation sites, N48 and T129, of HLA-DR1 beta chain are shown as sticks. CD4 (cyan), HLA-DRB1 (yellow), antigen peptide (magenta), and TCR (green)(lower right).

### (12) TIL Isolation Protocol

Tumors of SW1 or SM1 melanoma cells from C3H/HeJ or C57BL/6 mice, respectively) were digested using 1× tumor digest buffer (0.5 mg/mL Collagenase I, 0.5 mg/mL Collagenase IV, 0.25 mg/mL Hyalyronidase V, 0.1 mg/mL DNAse I in HBSS (Millipore Sigma (St. Louis, MO)). Tumors were homogenized using the Miltenyi MACs dissociator. Red blood cells were lysed using ACK lysis buffer (Life Technologies, (Grand Island, NY)). Tumor homogenate cells were counted using a standard hemocytometer.

### (13) Human Donor Peripheral CD4⁺T Cell Isolation Protocol

Human CD4⁺ T cells were isolated from fresh peripheral blood monocyte cells (PBMC) using a CD4⁺ T cell negative selection isolation kit (Stem Cell Technologies, (Vancouver CA)) according to manufacturer's protocols. CD4⁺T cells were cultured in the presence of vehicle or 250 μM L-fucose and were activated using anti-CD3/CD28 Dynabeads (ThermoFisher Scientific (Waltham, MA)) in a 1:1 bead:CD4⁺T cell ratio. After 48 h, cell pellets were collected and lysed for either lectin-based fucoproteomics or phosphoproteomics.

### (14) Flow Cytometry

(a) itIC and Splenic Profiling:

Total TILs were gated first to single cells (based on forward scatter height vs width, followed by side scatter height vs. width). Live cells were gated from the Zombie negative population from the population above. TILs were gated based on splenocyte size from a control spleen. Individual immune subpopulations were sub-gated from the total TIL population using the following staining criteria: CD3⁺ for CD3⁺ T cells; CD3⁺/CD4⁺/CD8⁻ for CD4⁺T cells; CD3⁺/CD4⁻/CD8⁺ for CD8⁺ T cells, CD11c⁺/CD11b⁺ for DCs; either NK1.1 (for C57/BL6 mice) or DX5 (for C3H/HeJ) for NK cells; CD11b⁺/GR1⁺ for MDSC-like cells; and F4/80⁺ for macrophages. Single-cell suspensions from tumor and spleen tissue were stained with Live/Dead Zombie NIR (Biolegend, (San Diego, CA)) at 1:1,000 in PBS for 20 min. Cell suspensions were spun down and stained with the following with antibodies at 0.5 μg/ml per antibody: APC anti-mouse CD3, Pacific Blue anti-mouse CD4, BV785 anti-mouse CD8, PerCP anti-mouse CD25, FITC anti-mouse F4/80, PeCy7 anti-mouse CD11c, PE anti-mouse NK1.1 or PE anti-mouse DX5, and PerCP-Cy5.5 anti-mouse CD11b. After staining, the cells were washed and fixed (2% formaldehyde), followed by another wash and flow cytometric analysis. The compensation controls were prepared using 0.5 μg/mL of each antibody with UltraComp eBeads, (ThermoFisher Scientific (Waltham, MA)). All samples were subject to flow cytometric profiling using a LSR Flow Cytometer (BD Biosciences (San Jose, CA)) and analysis as indicated using FlowJo software (BD Biosciences (San Jose, CA)).

### (b) Assessment of Cell Surface Fucosylation, HLA-DRB1, and PD-L1:

Indicated cells were treated for 72 h with DMSO, 250 μM fucosyltransferase inhibitor (FUTi) (Millipore Sigma (St. Louis, MO)), or 250 μM of L-fucose (Biosynth (Oak Terrace, IL)). After 72 h, cells were stained with 0.1 μM PKH26 (Millipore Sigma (St. Louis, MO)) prior to fixation in 4% formaldehyde solution. The cells were stained with anti-HLA-DRB1 and fluorescein AAL, or anti-human or anti-mouse PD-L1 overnight. The following day the cells were washed 3 times prior to adding AlexaFluor 594 donkey anti-rabbit. Cells were washed 3 times and then subject to flow cytometric analyses using a FACSCalibur (BD Biosciences (San Jose, CA)). Samples were analyzed using FlowJo analysis software (BD Biosciences (San Jose, CA)). Median values of DRB1 and AAL were normalized to PKH26 values and statistical analysis was performed using GraphPad Prism.

### (c) Assessment of Cell Surface Pan-MHC-I and Pan-MHC-H:

Surgically resected patient tumors were minced to less than 1-mm fragments. Minced tumor sample was enzymatically digested in enzyme media comprised of RPMI with collagenase type IV (1 mg/mL), DNase type IV (30 U/mL), and hyaluronidase type V (100 μg/mL) (Sigma). Single cell suspensions were strained through 40-micron nylon mesh and counted for viability via trypan blue exclusion, followed by cryopreservation for future analysis. Tumor homogenates were thawed and stained using Live/Dead Zombie NIR, PE anti-pan-MHC-I (HLA-A,B,C), FITC anti-pan-MHC-II, PerPCy5.5 anti-CD45, APC anti-CD90, and BV421 anti EpCAM. Flow cytometric data was analyzed using FlowJo analysis software (BD Biosciences (San Jose, CA)). MHC-I and MHC-II expression was dichotomized as positive or negative based on FMO samples for each marker. Statistical analysis was performed using GraphPad Prism.

### (15) Immunoprecipitation and Immunoblot Analyses

Cells were lysed on ice in RIPA lysis buffer (25 mM Tris-HCl pH 7.6, 150 mM NaCl, 5 mM EDTA, 1% NP-40 or 1% Triton X-100, 1% sodium deoxycholate, 0.1% SDS in diH₂O+protease and phosphatase inhibitors (ThermoFisher Scientific (Waltham, MA)), briefly sonicated, pelleted, and the resulting lysates were normalized by protein concentration using DC assay (BioRad Laboratories, (Hercules, CA)). The indicated samples were subjected to (12%) SDS-PAGE and immunoblot analysis using the indicated antibodies. Immunoblot imaging and analysis was performed using either an Odyssey FC scanner and ImageStudio (LiCor Biosciences, Lincoln, NE) or film.

(16) qRT-PCR

RNA from cells subjected to the indicated treatments was extracted using Gene Elute Mammalian Total RNA Extraction System (Millipore Sigma (St. Louis, MO)). RNA was reversed transcribed to cDNA using High-Capacity cDNA Reverse Transcription Kit (ThermoFisher Scientific (Waltham, MA)). qRT-PCR analysis was performed using FastStart Universal SYBR Green Master Mix (Rox) (Roche Diagnostics, (Indianapolis, IN)) using a BioRad CFX96 Real-time system (BioRad Laboratories, (Hercules, CA)). The qRT-PCR cycles used were as follows: 95° C. for 10 min, 35 cycles of 95° C. for 15 seconds, 55° C. for 60 seconds,

| | |
|---|---|
| Primer | Sequence (5′ −> 3′) |

Cloning Primers

| | |
|---|---|
| Fuk (mouse) | F: CGCGCGCGGGATCCATGGAGCAGTCAGAGGGAGTCAATTGGACTG (SEQ ID NO: 1) |
| | R: CGCGCGCGGCTAGCGGTGGTGCCCACTTCAGAGGGCC (SEQ ID NO: 2) |
| HLA-DRB1 | F: GTCTTTGAAGGATACACAGCCACCTTAGGATGGACTCG (SEQ ID NO: 3) |
| N48G | R: TGAGTGTCATTTCTTCGGTGGGACGGAGCGGG (SEQ ID NO: 4) |
| HLA-DRB1 | F: CGAGTCCATCCTAAGGTGGCTGTGTATCCTTCAAAGAC (SEQ ID NO: 5) |
| T129A | R: GTCTTTGAAGGATACACAGCCACCTTAGGATGGACTCG (SEQ ID NO: 6) |
| EB1 | F: CGCGCCCGGGCGCGCCATGGTGGTGTGGC (SEQ ID NO: 7) |
| | R: CGCGCCCGCTCGAGGCTCAGGAGTCC (SEQ ID NO: 8) |
| EB1 N46G | F: GTGTCATTTCTACGGCGGGACGCAGCGC (SEQ ID NO: 9) |
| | R: GCGCTGCGTCCCGCCGTAGAAATGACAC (SEQ ID NO: 10) |

qRT-PCR

| | |
|---|---|
| H3A (human | F: AAGCAGACTGCCGCAAAT (SEQ ID NO: 11) |
| and mouse) | R: GGCCTGTAACGATGAGGTTTC (SEQ ID NO: 12) |
| Fuk (mouse) | F: ACTTCCGCCGAGATCTGTTC (SEQ ID NO: 13) |
| | R: GGATCAGTGGACGTAGGCAG (SEQ ID NO: 14) |
| EB1 | F: GAACACGCTTCTTCCTTGGG (SEQ ID NO: 15) |
| | R: CAGGCTCCTTACCTTTCTGGT (SEQ ID NO: 16) |
| H2-K1 | F: CCGCGGACGCTGGATA (SEQ ID NO: 17) |
| | R: GGCGATTCGCGACTTCTG (SEQ ID NO: 18) |
| HLA-DRB1 | F: CCATAGTAGCTCAGCACCCG (SEQ ID NO: 19) |
| | R: GTCCTGTCCTGTTCTCCAGC (SEO ID NO: 20) |

and 72° C. for 30 seconds. Expression of the indicated genes was normalized to histone H3A expression. Primers for qRT-PCR were generated using NCBI primer blast software (National Center for Biotechnology Information (Washington, D.C.)) as detailed the table below.

### (17) Fluorescent Immunocytochemical and Immunohistological Staining and Analysis

General fluorescent immunocytochemical staining protocol: Melanoma cells were grown on German glass coverslips (Electron Microscopy Services (Hatfield, PA)) and fixed in fixation buffer (4% formaldehyde, 2% sucrose in phosphate buffered saline (PBS) for 20 min at room temperature (RT). The coverslip-grown cells were subject to two 5-min standing washes in PBS prior to permeabilization in permeabilization buffer (0.4% Triton-X-100 and 0.4% IgG-free bovine serum albumin (BSA, Jackson ImmunoResearch Laboratories (Westgrove, PA) in PBS) for 20 min at RT. The coverslip-grown cells were next subject to 2 PBS washes and incubated with the indicated primary antibodies.

### (a) General Fluorescent Immunohistochemical Tissue Staining Protocol:

In general, paraffin-embedded FFPE tumor tissue sections (or the TMA slide) were melted at 70° C. for 30 min. The slides were further de-paraffinizeded using xylene and rehydrated in serial alcohol washes. The slides were pressure cooked at 15 PSI for 15 min in a 1×DAKO antigen retrieval buffer (Agilent Technologies (Santa Clara, CA)). The tumor sections were subject to two 5-min standing washes in PBS prior to blocking in 1× Carb-Free Blocking Solution (Vector Labs (Burlingame, CA)) for 2-3h. The slides were next washed twice and incubated with indicated lectin and/or antibodies.

(b) General Fluorescent Analysis of Mouse Tumor Tissue fucosylation (FIGS. 8a, 8d, 8k):

For assessment of mouse tumor fucosylation, FFPE tumor sections were immunostained with FITC-conjugated AAL lectin (0.4 μg/mL, Vector Laboratories (Burlingame, CA)) and rabbit anti-Mart1+rabbit anti-S100 (melanoma marker cocktail). The slides were mounted with Vectashield+DAPI (Vector Laboratories (Burlingame, CA)). Four representative microscopy images per tumor were acquired using a Keyence BZ-X710, and images were process and analyzed using FUI (NIH) as follows: melanoma marker-positive regions were assigned as regions of interest (ROI) in which we measured Integrated density of AAL signal. Integrated densities of control tumors were assigned as 1, and Integrated AAL density values of experimental tumors were divided by control to produce relative fold changes and plotted as column charts.

### (c) Immunofluorescent Staining and Analysis of Melanoma Tissues and TMA (FIG. 1N):

### (i) Immunostaining and Image Acquisition:

Melanoma TMA (Serial #ME1002b; US BioMax, Inc. (Derwood, MD)) was immunostained with FITC-conjugated AAL lectin (0.4 μg/mL, Vector Laboratories (Burlingame, CA)), rabbit anti-Mart1, rabbit anti-S100, and anti-CD3 followed by AlexaFluor 568 (Cy3) donkey anti-rabbit and AlexaFluor 647 (Cy5) donkey anti-mouse secondary antibodies. The slides were mounted with Vectashield+DAPI (Vector Laboratories (Burlingame, CA)). An Aperio Scanscope FL (Leica Biosystems) was used to scan the TMA slide at 20× magnification and the digital slide saved into the Spectrum e-slide database.

### (ii) Analysis:

The multiplex fluorescence TMA image file was imported into Definiens Tissue Studio version 4.7 (Definiens AG, Munich, Germany), where individual cores were identified using the software's automated TMA segmentation tool. First, nucleus segmentation (DAPI channel) and cell growth algorithms were used to segment individual cells within each core. A minimum size threshold was used to refine the cell segmentation. Next, mean fluorescence intensity (MFI) values for the FITC (fucosylation), Cy3 (melanoma markers Mart1+S100) and Cy5 (CD3 marker) channels were extracted from each segmented cell and minimum thresholds for MFI was set to enumerate positive Cy3 and Cy5 cells. Identical thresholds were used for each core. Finally average MFI values for each core were reported for the FITC and Cy3 channels.

Melanoma-specific fucosylation (FITC in CY3-positive cells) MFI and CD3⁺ cell numbers were subject to statistical analyses and correlation with clinical parameters as follows: We used the nonparametric Wilcoxon rank sum test to compare melanoma-specific fucosylation levels between CD3⁺ T cells high vs low groups. The density values of CD3⁺ T cells were all log 2 transformed in the statistical analysis. Multivariable linear regression was used to assess the association between fucosylation and T cells while adjusting for confounding factors including sex, age and stage. The Spearman correlation coefficient was used to measure the correlation between melanoma-specific fucosylation and T cells in different sex groups.

### (18) Lectin-Mediated Proximity Ligation Assay (L-PLA)

Coverslip-grown cells subjected to L-PLA were processed upfront as described in the fluorescent immunocytochemistry protocol detailed above, whereas FFPE tumor tissue sections were processed according to the fluorescent immunohistochemistry protocol detailed above. Both approaches used mouse-anti-HLA-DRB1 (applied at 0.2 μg/mL, ab215835, Abcam, Cambridge, UK), biotinylated AAL lectin (applied at 0.2 μg/mL, Vector Laboratories (Burlingame, CA)), on coverslips overnight in 4° C. The coverslip-grown cells were again washed twice with PBS followed and then incubated with phalloidin Alexafluor 488 (applied at 0.05 μg/mL, ThermoFisher Scientific (Waltham, MA) with goat anti-biotin (applied at 0.1 μg/mL, Vector Laboratories (Burlingame, CA)) for 2h in 4° C. Subsequent steps of the protocol were adapted from the DUOlink In Situ Green PLA kit's manufacturer's protocol (Millipore Sigma (St. Louis, MO)). PLA anti-goat MINUS and PLA anti-mouse PLUS probes were applied at 1:5 for 1 h at 37° C. The coverslips were washed twice with Wash Buffer A prior to ligation with 1:5 ligation buffer and 1:40 ligase in ddH2O for 30 min at 37° C. The coverslips were washed twice with wash buffer A prior to incubation in amplification mix (1:5 amplification buffer and 1:80 polymerase in ddH2O for 1.5 h at 37° C.). Coverslips were washed twice with Wash Buffer B prior to mounting to slide with DAPI with VectaShield (Vector Labs, Burlingame, CA). Microscopy images were acquired using a Keyence BZ-X710, and images were process and analyzed using FIJI (NIH).

### (19) Immunofluorescent Staining, Image Acquisition, and Analysis of Anti-PD1-Treated Melanoma Patients (FIG. 5c):

The indicated FFPE sections were immunostained with anti-DRB1 antibody or L-PLA stained as detailed above with the addition of anti-CD4⁺ antibody. WTS imaging was performed using the Vectra3 Automated Quantitative Pathology Imaging System (PerkinElmer, Waltham, MA). 20×ROI tiles were sequentially scanned across the slide and spectrally unmixed using InForm (PerkinElmer, Waltham, MA) and the multilayer Tiff files were exported. HALO (indica labs, Albuquerque, NM) was used to fuse the tile images together prior to WTS image analysis. For each whole tumor image, (i) every individual melanoma marker (MART1+S100)-positive cell was segmented and quantitatively measured for total fucosylation, total HLA-DRB1, and fucosylated HLA-DRB1, and (ii) every CD4⁺T cell within the melanoma marker-positive tissue region was counted. Per patient (Pt.), these marker values were box plotted to visualize the staining distribution of individual tumor cells. The total numbers of melanoma cells per patient section measured and analyzed were as follows: Pt. 1: 557,146 cells; Pt. 2: 743,172 cells; Pt. 3: 95,628 cells; and Pt. 4: 13,423 cells.

### (20) Anti-PD1-Treated Patient Specimens (FIGS. 5d & 5e, and EXTENDED DATA FIGS. 4D & 4E)

Moffitt Cancer Center patient specimens: Patients with advanced stage melanoma being treated at Moffitt Cancer Center were identified, and specimens collected and analyzed following patient consent under Moffitt Cancer Center Institutional Review Board approved protocols.

For FIGS. 5d & 5e: De-identified Moffitt “Responder” patients exhibited greater than 20 months of progression-free survival, whereas “Non-Responder” patients progressed in less than 6 months after receiving anti-PD1.

For FIGS. 11D & 11E: Non-response status to PD1 checkpoint blockade therapy (nivolumab or pembrolizumab) was defined as progression of disease by RECIST 1.1 while on PD-1 checkpoint blockade therapy or within 3 months of last dose.

MD Anderson Cancer Center patient specimens: Biospecimens were retrieved, collected and analyzed after patient consent under UT MD Anderson Cancer Center Institutional Review Board-approved protocols. Patients with advanced (stage III/IV) melanoma treated at The University of Texas MD Anderson Cancer Center between 07/01/2015 and 05/01/2020 who received at least one dose of PD-1 checkpoint blockade agent (either nivolumab or pembrolizumab) were identified from detailed retrospective and prospective review of clinic records. Responder status was defined as a complete or partial response and non-responder was defined as stable or progressive disease by RECIST 1.1. Pathologic response was defined by the presence or absence of viable tumor on pathologic review when available.

Massachusetts General Hospital patient specimens: Patients initiating anti-PD1 (Pembrolizumab) as front-line treatment for metastatic melanoma at MGH provided written informed consent for the collection of tissue and blood samples for research and genomic profiling (DF/HCC IRB approved Protocol 11-181). Patients classified as responders (R) showed clear radiographic decrease in disease at initial staging through a minimum of 12 weeks. Patients classified as non-responders (NR) did not respond to treatment radiographically and/or had clear and rapid progression. Progression free survival (PFS) is given in days from treatment start to radiographic scan when progression was first noted (uncensored) or last progression free scan (censured). Overall survival (OS) is given in days from treatment start to date of death (uncensored) or last follow-up (censored).

### (21) Animal Models

All animals were housed at the Vincent A. Stabile Research building animal facility at H. Lee Moffitt Cancer Center & Research Institute, which is fully accredited by the Association for Assessment and Accreditation of Laboratory Animal Care International (AAALAC, #434), and are managed in accordance with the Guide for the Care and Use of Laboratory Animals (“The Guide”), the Animal Welfare Regulations Title 9 Code of Federal Regulations Subchapter A, “Animal Welfare”, Parts 1-3 (AWR), the Public Health Service Policy on Humane Care and Use of Laboratory Animals (PHS Policy), and by the USF Institutional Animal Care and Use Committee's Principles and Procedures of Animal Care and Use (IACUC Principles). The experiments and protocols detailed in this study received institutional approval by the Moffitt IACUC (RIS00001625). Four-to-six-week-old female C3H/HeN and male C57BL6 mice were purchased from Charles Rivers Laboratories for the indicated experiments. Four-to-six-week-old male NSG mice from the Lau laboratory breeding colony were used for the indicated experiments. Power calculations were used to determine mouse cohort sizes to detect significant changes in tumor sizes. In general, 10 mice per indicated cohort to accommodate for incidental loss of mice due to issues beyond our control (e.g., incidental tumor ulceration that required exclusion from the study). Mouse tumor volumes were measured using length, width and depth divided by 2. At each experimental endpoint, mice were humanely euthanized using CO₂ inhalation in accordance to the American Veterinary Medical Association guidelines. Mice were observed daily and humanely euthanized if the tumor reached 2,000 mm³ or mice showed signs of metastatic disease.

For all mouse models, 1×10⁶ melanoma cells were injected subcutaneously in the right hind flanks of each mouse. Between 7-14 days, when the tumor volumes reached −150 mm³, the mice were either supplemented with or without 100 mM L-fucose (Biosynth (Oak Terrace, IL)) via drinking water, which was provided ad libitum and which we demonstrated to increase tumor fucosylation and to suppress melanomas. This dosage is within ranges for dietary supplementation with L-fucose and other similar dietary sugars (e.g., D-mannose) in other rodent studies. When the tumors reached −2 cm³, the animals were sacrificed, and the tumors either processed for flow cytometric profiling or for histological analysis as indicated.

Control vs. mFuk±L-fucose models (FIG. 1 & FIG. 8): SW1 or SM1 mouse melanoma cells were injected into syngeneic C3H/HeN (or NSG) female or C57BL/6 male mice, respectively, as follows: parental SW1 cells for FIG. 1A; parental SM1 cells for FIG. 1E; SW1 cells stably expressing either empty vector (EV) or mouse fucose kinase (mFuk) for FIG. 1L; and parental SW1 cells for FIG. 1M.

Control vs. L-fucose±FTY720 models (FIG. 2): SW1 or SM1 mouse melanoma cells were injected into syngeneic C3H/HeN (or NSG) female or C57BL/6 male mice, respectively. Cells were injected as follows: parental SW1 cells for FIG. 1A; parental SM1 cells for FIG. 1E; SW1 cells stably expressing either empty vector (EV) or mouse fucose kinase (mFuk) for FIG. 1L; and parental SW1 cells for FIG. 1M. FTY720 was administered at 20 μg every 2 days starting on Day 12, just prior to the initiation of LF, until endpoint.

Immunodepletion mouse models (FIG. 1 & FIG. 8): Three days prior to tumor engraftment, PBS (control) or ˜300 μg α-CD4 (20 mg/kg, for immunodepletion, GK1.5, Bioxcell (West Lebanon, NH)) or 0-CD8 (20 mg/kg, for immunodepletion, 2.43, Bioxcell (West Lebanon, NH)) was administered by intraperitoneal injection into the indicated cohorts of mice. Injections of immunodepletion antibody or PBS were continued every 3-4 days until endpoint. Syngeneic recipient C3H/HeN female or C57BL/6 male mice were injected with SW1 or SM1 cells, respectively.

HLA-A/HLA-DRB1 knockdown and glyco-fucomutant H2-EB1 reconstitution mouse model (FIGS. 2 & 3): SW1 mouse melanoma cells expressing either shNT (non-targeting shRNA), shH2K1, shEB1, shNT+EV, shEB1+EV, shEB1+EB1 WT, or shEB1+EB1 N46G were injected into syngeneic C3H/HeN female mice.

anti-PD-1 mouse model (FIG. 4): SW1 or SM1 mouse melanoma cells were injected into syngeneic C3H/HeN female or C57BL/6 male mice, respectively. After approximately 7 days, when the mice tumors reached −150 mm³, the mice were either supplemented with or without 100 mM L-fucose (Biosynth (Oak Terrace, IL)) via drinking water, which was provided ad libitum. Simultaneously, PBS (control) or anti-PD1 (20 mg/kg, clone RMP1-14, Bioxcell (West Lebanon, NH)) were administered via intraperitoneal injection every 3-4 days until endpoint. Mice were sacrificed, and tumors and indicated organs were harvested for analysis at indicated timepoints.

NSG melanoma model (FIG. 1 model): SW1 murine mouse melanoma cells were subcutaneously injected into the right rear flanks of NSG mice.

### (22) Quantification and Statistical Analysis

GraphPad Prism was used for statistical calculations unless otherwise indicated. For all comparisons between 2 independent conditions, t tests were performed to obtain p values and standard error of the mean (SEM). For comparisons between ≥2 groups, one way or two-way ANOVAs were performed, and p values and SEMs were obtained. For the TMA data, Wilcoxon signed-rank test was used to determine significance.

### 2. Example 2: Androgen- and Fucosylation-Regulated Invasiveness: A Key Molecular Mechanism Underpinning Disparate Sex-Associated Invasiveness and Metastasis in Melanoma

The incidence and mortality rates of melanoma are historically higher for men than women, with an estimated ˜34% more new cases and twice the lethality in men in the US in 2022. Although emerging studies have highlighted roles for the male sex hormone androgen and its receptor (AR) in melanoma proliferation, motility, and therapeutic resistance, the underlying cellular and molecular mechanisms are not well-defined.

We discovered the sex-associated disparity in melanoma fucosylation, which is a type of post-translational modification of glycoproteins with the dietary sugar L-fucose (L-fuc). Fucosylation can promote or suppress tumors-divergent functions dictated by 13 tumor-promoting or tumorsuppressing fucosyltransferases (FUTs) that conjugate fucose moieties onto targeted proteins. However, there is currently a knowledge gap regarding the association between androgen/AR signaling and fucosylation in melanoma.

In this study, we delineated a novel relationship between androgen signaling and fucosylation network in driving melanoma malignancy. Herein we advanced the understanding of sex-associated discrepancies in melanoma and enhanced clinical stratification for personalized melanoma treatment.

In the cytosol, free L-Fuc is phosphorylated by fucokinase (FUK) and GDP-coupled by fucose-1-phosphate guanylyltransferase (FPGT) to yield GDP-fucose, which is the global substrate for cellular protein fucosylation. Synthesized GDP-fucose is transported into ER/Golgi via SLC35C1/2 transporters, where fucose moieties are conjugated onto proteins by 13 FUTs. FUK, FPGT, and SLC35C1/2 regulate the availability of global substrate (GDPfucose), whereas the FUTs are rate-limiting and determine the tumorpromoting vs. tumor-suppressive subtype of fucosylation.

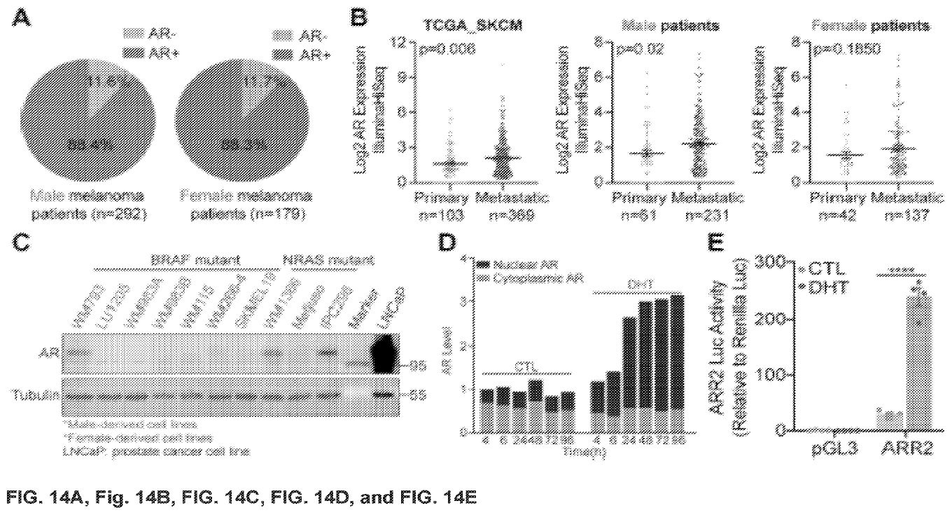

FIGS. 14A, 14B, 14C, 14D, and 14E show that melanoma cells express androgen-inducible and transcriptionally active AR. FIG. 14A shows AR expression in male and female melanoma tissues from TCGA skin cutaneous melanoma (SKCM) dataset. FIG. 14B shows AR expression in primary and metastatic melanomas from TCGA SKCM dataset. FIG. 14C shows immunoblotting analysis of baseline AR protein level across 10 melanoma cell lines. FIG. 14D shows nuclear fractionation followed by immunoblotting of AR protein in WM793 cells±100 nM dihydrotestosterone (DHT) over 96 hours. FIG. 14E shows AR binding motif-containing promoter (ARR2) luciferase assay on WM793 cells±100 nM DHT.

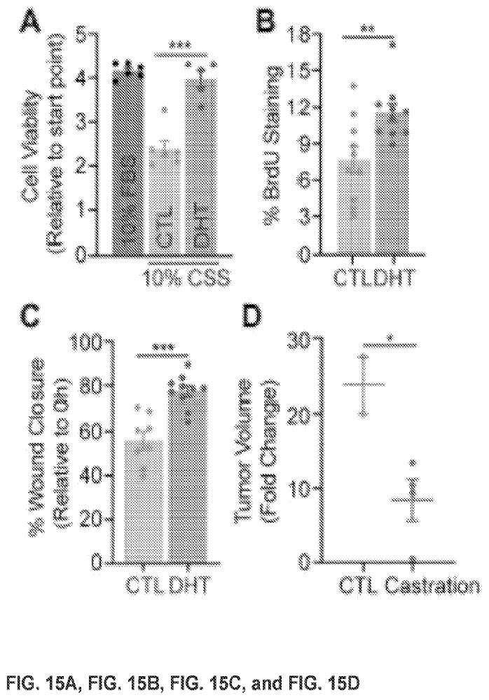

FIGS. 15A, 15B, 15C, and 15D show the biological functions of androgen in melanoma. FIGS. 15A-15C shows (FIG. 15A) MTT, (15B) BrdU, and (FIG. 15C) Wound healing assays for WM793 cells±100 nM DHT. FIG. 15D shows the fold change of tumor volume in C57BL/6-SM1 mice model at the end point (35d after implantation). Mice were castrated 1.5 weeks prior to injection.

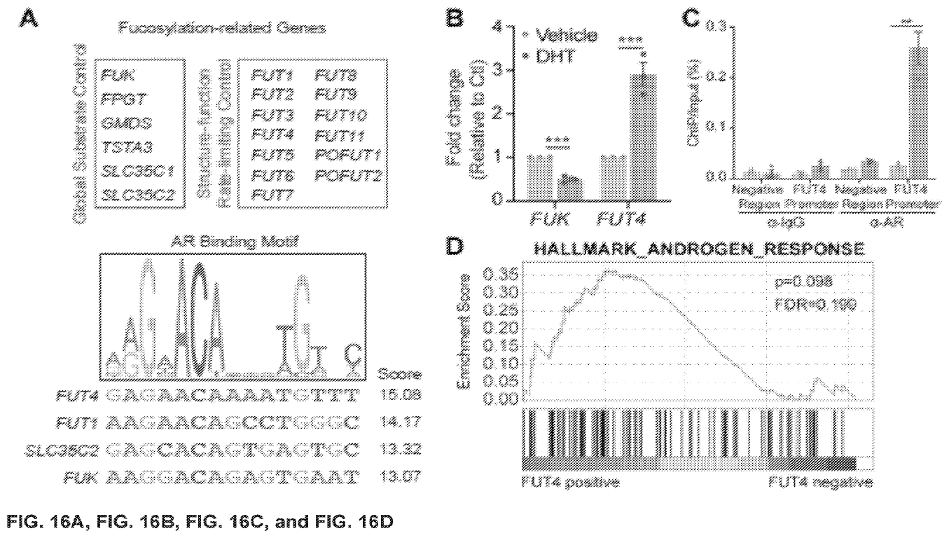

FIGS. 16A, 16B, 16C, and 16D show that AR transcriptionally upregulates FUT4 expression via binding to the ARE motif in FUT4 promoter. FIG. 16A shows the predicted AR-binding sites in the promoter of FUT4, FUT1, SLC35C2, and FUK genes. FIG. 16B shows qRT-PCR assessing mRNA levels of FUK and FUT4 altered by DHT treatment in WM793 cells. FIG. 16C shows ChIPqPCR analysis of the enrichment of AR protein at −515-502 bp promoter region of FUT4 gene upon DHT treatment. FIG. 16D shows hallmark GSEA associates FUT4 expression with androgen response gene signatures in TCGA SKCM samples.

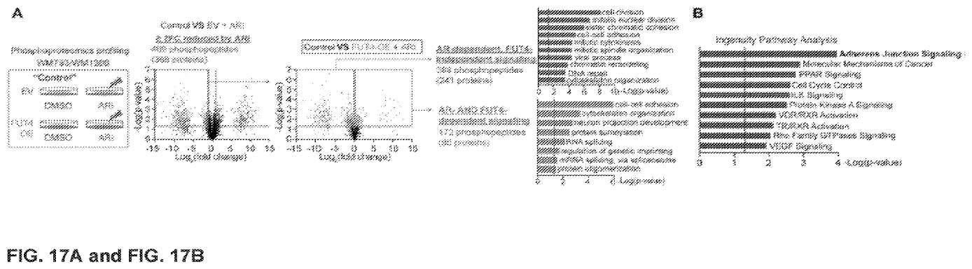

FIGS. 17A and 17B show AR-FUT4-dependent signaling regulates cell adhesion/motility, whereas AR-dependent/FUT4-independent signaling regulates cell division. FIG. 17A shows phosphoproteomics profiling of EV/FUT4-OE melanoma cells±AR inhibitor. Pathway enrichment analyses were performed on DAVID (Functional Annotation Tool). FIG. 17B shows ingenuity pathway analysis (IPA) listed adherens junctions (AJs) as the top 1 AR/FUT4-regulated signaling.

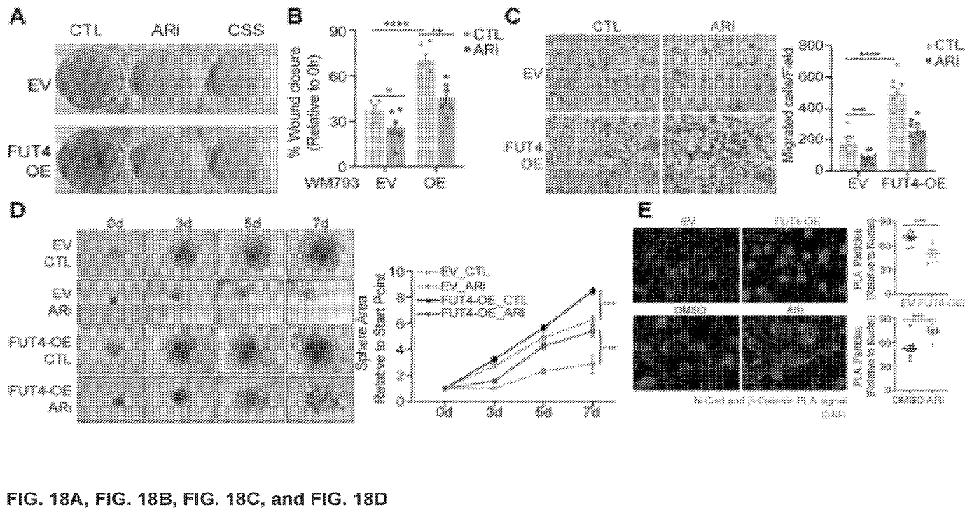

FIGS. 18A, 18B, 18C, 18D, and 18E show AR-FUT4 axis facilitates melanoma invasion via disrupting N-cadherin/catenin junction complexes. FIG. 18A shows clonogenic assay on WM793 cells±10 uM AR inhibitor or ±cultured in charcoal-stripped serum (CSS). FIG. 18B shows wound healing assay, (FIG. 18C) Matrigel invasion assay, and (FIG. 18D) 3D spheroid cell invasion assay on EV/FUT4-OE WM793 cells±10 uM AR inhibitor. FIG. 18E shows proximity ligation assay evaluating the interaction of N-cadherin and β-catenin proteins in EV/FUT4-OE WM793 cells and parental WM793 cells±10 uM AR inhibitor.

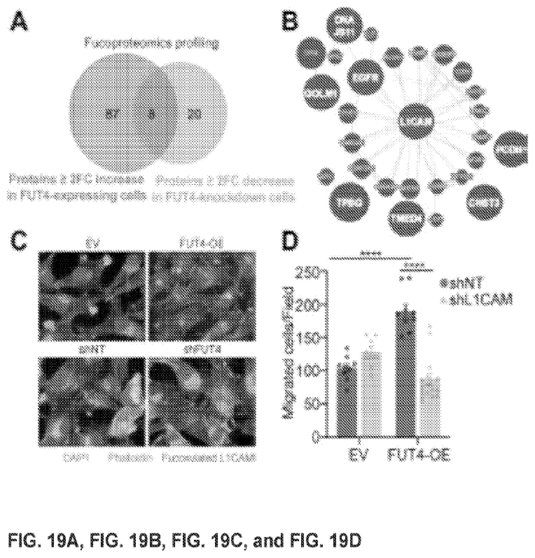

FIGS. 19A, 19B, 19C, and 19D show FUT4-fucosylated L1CAM is required for AR-FUT4-induced melanoma invasiveness. FIG. 19A shows fucoproteomics profiling of WM793 cells ectopically expressing FUT4. FIG. 19B shows GeneMania interactome mapping of eight protein hits. FIG. 19C shows lectin proximity ligation assay on EV/FUT4-OE and shNT/shFUT4 WM793 cells. FIG. 19D shows matrigel invasion assay on FUT4 and L1CAM double-modified WM793 cells.

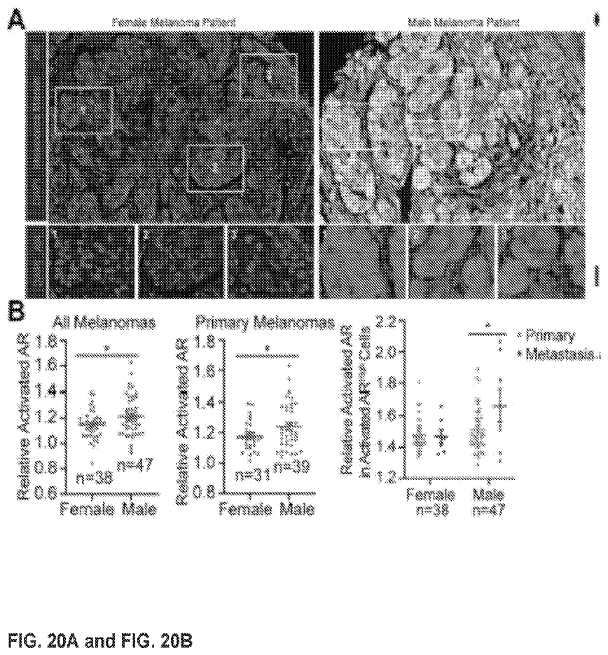

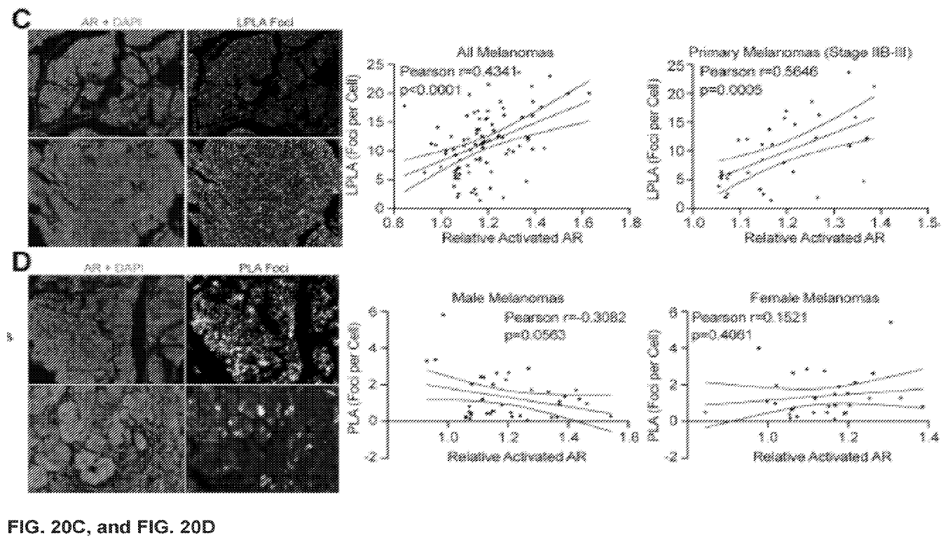

FIGS. 20A, 20B, 20C, and 20D show the activation of AR-FUT4-L1CAM-AJs signaling axis in male melanomas. FIG. 20A shows representative pictures of multiplexed immunofluorescence-stained melanoma TMA (#ME1004h). FIG. 20B shows (left) The level of relative activated AR (the ratio of nuclear AR/cytoplasmic AR) between female and male melanomas. (right) The level of activated AR in ARhigh melanoma cell population between primary and metastatic melanomas. FIG. 20C shows the Correlation analysis of activated AR & fucosylated-L1CAM (LPLA Foci) as well as (FIG. 20D) of activated AR & N-Cad/0-catenin junction complexes (PLA Foci).

Melanoma cells are variably sensitive to sex hormone androgen attributed to endogenous AR levels. Androgen-activated AR signaling plays a crucial role in driving melanoma metastasis by inducing FUT4 expression. AR-FUT4 axis enhances cell motility by disrupting Ncadherin-mediated cell-cell adhesion. Delineate the contributions of AR/FUT4/L1CAM axis to melanoma metastasis in vivo (mice models) Fucosylated L1CAM is an essential downstream effector of AR-FUT4 axis

### 3. Example 3: Leveraging L-Fucose-Mediated Signaling to Induce Monocyte-Derived Dendritic Cell Polarization

Dietary sugar L-fucose (L-fuc), when co-administered with the immune checkpoint blockade agent anti-PD1, significantly enhances anti-tumor immunity and tumor suppression compared to single agent anti-PD1 in mouse models of melanoma. Further, we found that concomitant with increased tumor suppression, dietary L-fuc supplementation also increases the number of intratumoral myeloid cells (MCs) in mouse models of melanoma and breast cancer. Intriguingly, we found that L-fuc treatment of bone marrow-derived MCs (BMMCs) (i) increases their T cell-stimulatory activity (ex vivo co-culture/IFNγ assays), and (ii), polarizes MCs towards the monocyte-derived dendritic cell (moDC) phenotype without affecting absolute DC numbers (flow cytometric profiling). We also observed that “L-fuc-induced moDCs” exhibit more abundant and longer dendrites than untreated DCs, suggesting a more dynamic cytoskeleton as well as reduction in immunosuppressive molecules. Consistent with this notion, L-fuc-induced moDCs uptake exogenous Fitc-dextran bait at a significantly increased rate compared with untreated DCs, suggesting that the increased and longer dendrites may enhance antigen uptake. Our preliminary studies have begun to dissect and highlight moDC signaling changes induced by L-fuc treatment. Here, we present and discuss our novel findings on L-fuc-induced DC/moDC biology, the key mechanistic signaling changes that mediate these effects, and the important translational implications of these findings for efforts aimed at increasing/enhancing intratumoral DC function and anti-tumor immune responses.

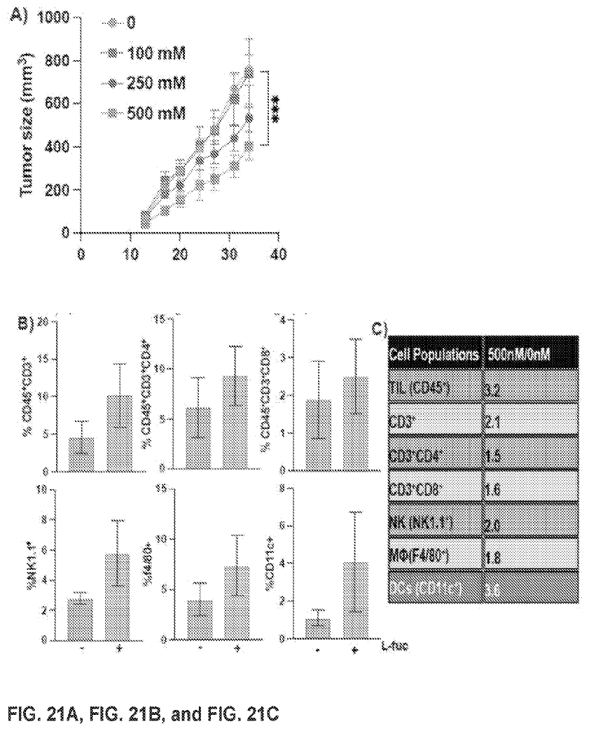

We observed that L-fucose reduces tumor growth in breast cancer and enriches for CD11c+ cells. To determine the effect of L-fucose treatment on a less immunologically active tumor we used a syngeneic breast tumor model and measured the dose-dependent tumor suppression (FIG. 21A). We found that L-fucose treatment of breast tumors leads to dose-dependent tumor suppression. Next, we wanted to determine which immune populations may be contributing to the reduced tumor growth we next harvested the tumors and performed flow cytometry to identify common subsets of immune cells (T cells, NK cells, DCs and macrophages (21B) we then compared the change between the 500 mM L-fucose treated group to the control group to determine which populations had the highest overall change (21C). It was found that L-fucose treated breast tumors show an enrichment of CD11c+ cells. We hypothesized that L-fucose bolsters the antitumor immune response by altering dendritic cell functionality.

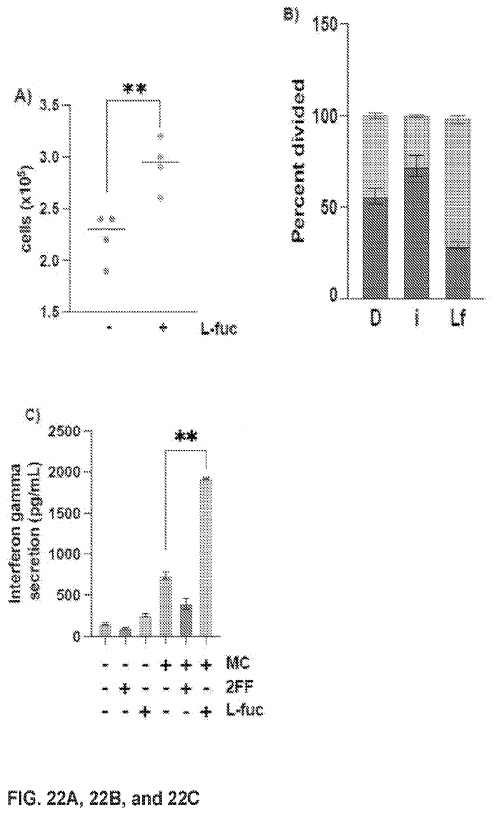

To assess whether the effect in DC enrichment after L-fucose resulted in altered DC biology we isolated immature myeloid cells from BM to (FIG. 22A) confirm that CD11c+ population was enriched. We next sought to see how the immunostimulatory capacity of these cells was altered after L-fucose treatment by culturing L-fucose modulated, either by decreased fucosylation via 2FF or increased fucosylation by L-fuc, MCs with immature T cells to asses (FIG. 22B) proliferation by loss of CTV staining and (FIG. 22C) T cell activation as evident by interferon-gamma release. We concluded that modulation of BMMC fucosylation alters DC immunostimulatory capacity ex vivo and L-fucose treated dendritic cells (DCs) show enhanced immunostimulatory capacity.

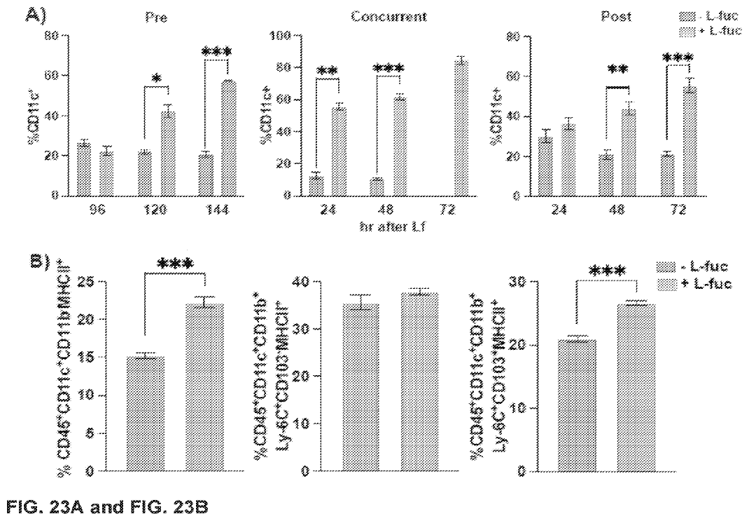

We next investigated the effect of L-fucose on dendritic cell differentiation. To test whether L-fucose affected immature or mature myeloid cells preferentially, BMMC were treated with L-fucose at various stages of development, pre=before maturation cocktail, concurrent=during maturation cocktail or post=after maturation cocktail (FIG. 23A). We found that L-fucose treatment leads to DC polarization of BMMC at any stage of myeloid development. We next sought to determine if L-fucose equally affected subpopulations of DCs. By flow cytometric analysis we were able to determine that moDC are affected most by the modulation of fucosylation in immature myeloid cell populations. As shown in FIG. 23B, L-fucose polarizes myeloid cells towards a moDC phenotype.

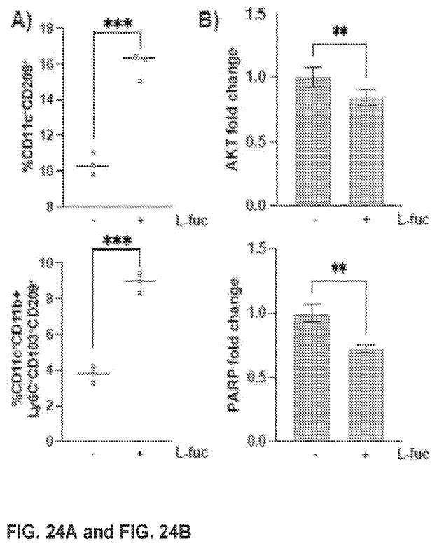

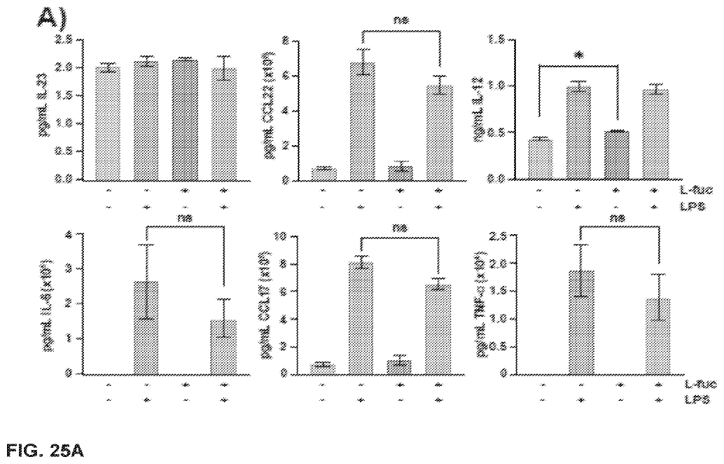

To better understand the phenotype of DC subsets after L-fucose treatment, we preformed broad panel analysis to highlight unique subsets of DCs, including markers of activation, cross presentation and known signaling mechanisms. Interestingly, we observed an increase in CD209 expression indicating the activation of this signaling pathway after L-fucose treatment (FIG. 24A). To determine if this increased expression was related to any other signaling changes we performed a signaling array on key phosphorylated proteins on activated DCs after L-fucose treatment. Among several changes we observed significant decreases in proteins that are known to produce immunosuppressive effects in myeloid cells, AKT and PARP, indicating a protective mechanism (FIG. 24B). From this, we concluded that L-fucose increases expression of CD209 and modulates downstream signaling paths

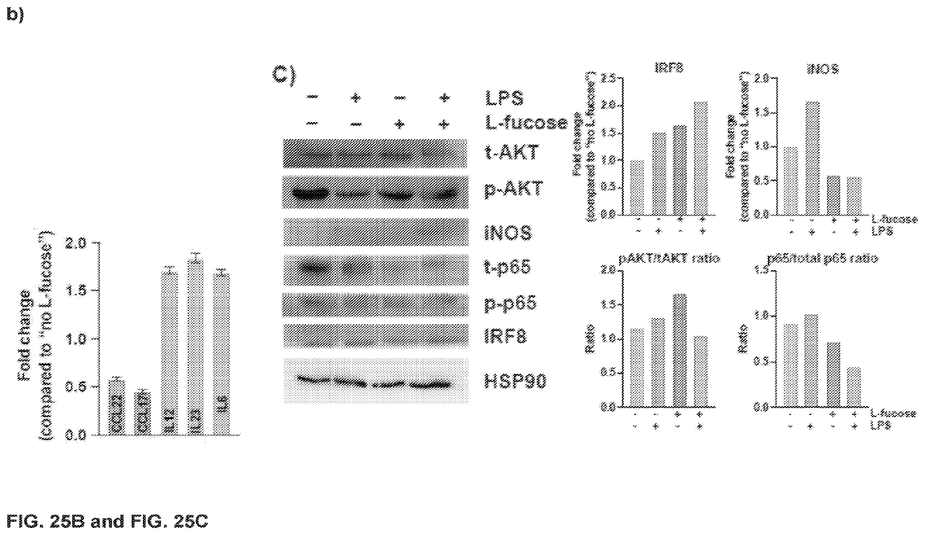

Next we investigated whether L-fucose treated DCs exhibit differential signaling pathways. DCs treated with L-fucose show decreased immunosuppressive signature. To determine if the changes in DC polarization and CD209 expression were associated with differential signaling changes in moDCs we analyzed known suppressive and stimulatory pathways by (FIG. 25A) cytokine production and (FIG. 25B) qPCR. Together these data suggest a loss of immunosuppressive activity and enhanced immunostimulation. As shown in FIG. 25C, changes in L-fucose treated moDC signaling is associated with decreased iNOS and p65 activation. (C) To elucidate the mechanism by which L-fucose was repressing immunosuppression in moDCs we preformed immunoblot analysis of several key signaling pathways in moDC activation of cells treated +/−L-fucose and +/−maturation via LPS. Additionally, we identified transcription factors that may play a role in altering the signaling and cytokine profiles of moDC to further validate the changes in immunomodulatory activity we observe.

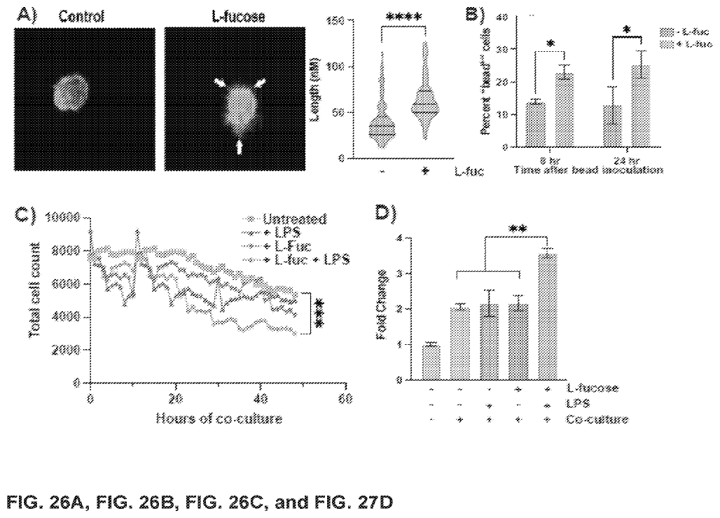

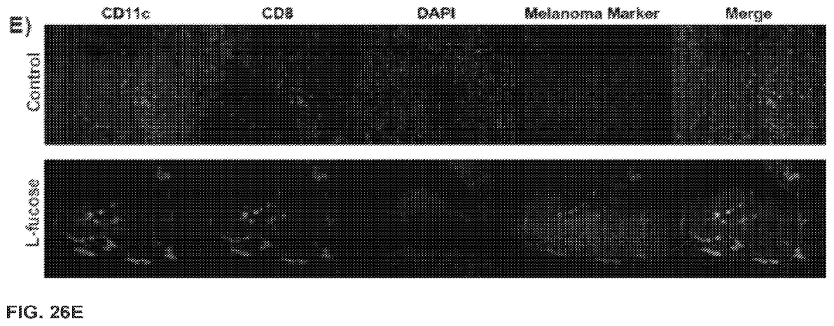

Next we wanted to determine if L-fucose enhances the function phenotype of moDCs. FIGS. 26A and 26B show that moDCs treated with L-fucose exhibit increased dendrite length and phagocytosis. Having determined that L-fucose alters the signaling of moDCs leading to an immunostimulatory phenotype, we next sought to identify other components of DC biology that are affected. To this end we examined L-fucose-treated mature dendritic cells for changes in (FIG. 26A) dendrite length and (FIG. 26B) phagocytosis by neutral bead uptake which may correlate with differential anti-tumor capacity in the TME.

Investigating this further we found that moDCs exhibits a cytotoxic-like phenotype after L-fucose treatment (FIGS. 26C and 26D). We next sought to determine if the change in phagocytosis we observe in the bead uptake assay was accompanied with increased cytotoxic tendencies in the L-fucose-treated moDCs. To this end we co-cultured pretreated moDCs with GFP-expressing SW1 tumor cells to observe (FIG. 26C) loss of GFP fluorescence correlating with SW1 and (FIG. 26D) release of LDH as markers of cell death. We also showed that L-fucose treatment is associated with increased clustering in vivo by observing SW1 tumors from mice treated +/−L-fucose had increased DC clustering by CD11c (in green).

Thus, we show that L-fucose reduces the tumor growth of an immunologically suppressed tumor and increases the abundance of myeloid cells in the tumor microenvironment.

We also show that L-fucose treatment promotes the polarization of moDC in the myeloid compartment moDCs treated with L-fucose show enhanced immunostimulatory activity in terms of antigen uptake, cytokine production, T cell stimulation and cell killing. Lastly, we show that the mechanism of L-fucose triggered enhanced moDC functionality is mediated by CD209 signaling activation and reduction in p65 activity.

### D. REFERENCES

Adhikari, E. et al. L-fucose, a sugary regulator of antitumor immunity and immunotherapies. Mol Carcinog (2022).
Alam, M.S. Proximity Ligation Assay (PLA). Curr Protoc Immunol 123, e58 (2018).
Alatrash, G. et al. Fucosylation Enhances the Efficacy of Adoptively Transferred Antigen-Specific Cytotoxic T Lymphocytes. Clin Cancer Res 25, 2610-2620 (2019).
Anderson, K.S. & Cresswell, P. A role for calnexin (IP90) in the assembly of class II MHC molecules. EMBO J 13, 675-682 (1994).
Bajana, S. et al. Differential CD4(+) T-cell memory responses induced by two subsets of human monocyte-derived dendritic cells. Immunology 122, 381-393 (2007).
Bastian, K., Scott, E., Elliott, D.J. & Munkley, J. FUT8 Alpha-(1,6)-Fucosyltransferase in Cancer. Int J Mol Sci 22 (2021).
Binnewies, M. et al. Unleashing Type-2 Dendritic Cells to Drive Protective Antitumor CD4(+) T Cell Immunity. Cell 177, 556-571 e516 (2019).
Chacon, J. A. et al. Manipulating the tumor microenvironment ex vivo for enhanced expansion of tumor-infiltrating lymphocytes for adoptive cell therapy. Clin Cancer Res 21, 611-621 (2015).
Chang, C. S., Brossay, L., Kronenberg, M. & Kane, K. P. The murine nonclassical class I major histocompatibility complex-like CD1.1 molecule protects target cells from lymphokine-activated killer cell cytolysis. J Exp Med 189, 483-491 (1999).
Cox, J. & Mann, M. MaxQuant enables high peptide identification rates, individualized p.p.b.-range mass accuracies and proteome-wide protein quantification. Nat Biotechnol 26, 1367-1372 (2008).
Das, M., Ithychanda, S., Qin, J. & Plow, E.F. Mechanisms of talin-dependent integrin signaling and crosstalk. Biochin Biophys Acta 1838, 579-588 (2014).
del Pozo, M. A., Sanchez-Mateos, P., Nieto, M. & Sanchez-Madrid, F. Chemokines regulate cellular polarization and adhesion receptor redistribution during lymphocyte interaction with endothelium and extracellular matrix. Involvement of cAMP signaling pathway. J Cell Biol 131, 495-508 (1995).
Etzioni, A. & Tonetti, M. Fucose supplementation in leukocyte adhesion deficiency type II. Blood 95, 3641-3643 (2000).
Fleury, S. et al. HLA-DR polymorphism affects the interaction with CD4. J Exp Med 182, 733-741 (1995).
Gay, D. et al. Functional interaction between human T-cell protein CD4 and the major histocompatibility complex HLA-DR antigen. Nature 328, 626-629 (1987).
Gellrich, F. F., Schmitz, M., Beissert, S. & Meier, F. Anti-PD-1 and Novel Combinations in the Treatment of Melanoma—An Update. J Clin Med 9 (2020).
Gentles, A. J. et al. The prognostic landscape of genes and infiltrating immune cells across human cancers. Nat Med 21, 938-945 (2015).
Grabinger, T. et al. Alleviation of Intestinal Inflammation by Oral Supplementation With 2-Fucosyllactose in Mice. Front Microbiol 10, 1385 (2019).
Hashim, O. H., Jayapalan, J. J. & Lee, C. S. Lectins: an effective tool for screening of potential cancer biomarkers. PeerJ 5, e3784 (2017).
Johnson, D. B. et al. Melanoma-specific MHC-II expression represents a tumour-autonomous phenotype and predicts response to anti-PD-1/PD-L1 therapy. Nat Conmun 7, 10582 (2016).
Kagamu, H. et al. CD4(+) T-cell Immunity in the Peripheral Blood Correlates with Response to Anti-PD-1 Therapy. Cancer Immunol Res 8, 334-344 (2020).
Keeley, T.S., Yang, S. & Lau, E. The Diverse Contributions of Fucose Linkages in Cancer. Cancers (Basel) 11 (2019).
Kennedy, R. & Celis, E. Multiple roles for CD4⁺T cells in anti-tumor immune responses. Immunol Rev 222, 129-144 (2008).
Keren, L. et al. A Structured Tumor-Immune Microenvironment in Triple Negative Breast Cancer Revealed by Multiplexed Ion Beam Imaging. Cell 174, 1373-1387 e1319 (2018).
Knight, D. A. et al. Host immunity contributes to the anti-melanoma activity of BRAF inhibitors. J Clin Invest 123, 1371-1381 (2013).
Kramer, A., Green, J., Pollard, J., Jr. & Tugendreich, S. Causal analysis approaches in Ingenuity Pathway Analysis. Bioinformatics 30, 523-530 (2014).
Kumari, S. et al. Cytoskeletal tension actively sustains the migratory T-cell synaptic contact. EMBO J 39, e102783 (2020).
Lau, E. et al. The transcription factor ATF2 promotes melanoma metastasis by suppressing protein fucosylation. Sci Signal 8, ra124 (2015).
Lenertz, L. Y. et al. Mutation of putative N-linked glycosylation sites on the human nucleotide receptor P2X7 reveals a key residue important for receptor function. Biochemistry 49, 4611-4619 (2010).
Liang, W. et al. Core Fucosylation of the T Cell Receptor Is Required for T Cell Activation. Front Immunol 9, 78 (2018).
Lim, C. J. et al. Integrin-mediated protein kinase A activation at the leading edge of migrating cells. Mol Biol Cell 19, 4930-4941 (2008).
Maletzki, C. et al. NSG mice as hosts for oncological precision medicine. Lab Invest 100, 27-37 (2020).
Marquardt, T. et al. Correction of leukocyte adhesion deficiency type II with oral fucose. Blood 94, 3976-3985 (1999).
Marth, J. D. & Grewal, P. K. Mammalian glycosylation in immunity. Nat Rev Immunol 8, 874-887 (2008).
Nestarenkaite, A. et al. Immuno-Interface Score to Predict Outcome in Colorectal Cancer Independent of Microsatellite Instability Status. Cancers (Basel) 12 (2020).
Nonaka, M. et al. Mannan-binding protein, a C-type serum lectin, recognizes primary colorectal carcinomas through tumor-associated Lewis glycans. J Immunol 192, 1294-1301 (2014).
Nordenfelt, P., Elliott, H.L. & Springer, T.A. Coordinated integrin activation by actin-dependent force during T-cell migration. Nat Commun 7, 13119 (2016).
Norton, P. et al. Development and application of a novel recombinant Aleuria aurantia lectin with enhanced core fucose binding for identification of glycoprotein biomarkers of hepatocellular carcinoma. Proteomics 16, 3126-3136 (2016).
Oberprieler, N. G. et al. High-resolution mapping of prostaglandin E2-dependent signaling networks identifies a constitutively active PKA signaling node in CD8⁺ CD45RO+ T cells. Blood 116, 2253-2265 (2010).
Oh, D. Y. et al. Intratumoral CD4(+) T Cells Mediate Anti-tumor Cytotoxicity in Human Bladder Cancer. Cell 181, 1612-1625 e1613 (2020).
Okada, M. et al. Blockage of Core Fucosylation Reduces Cell-Surface Expression of PD-1 and Promotes Anti-tumor Immune Responses of T Cells. Cell Rep 20, 1017-1028 (2017).
O'Malley, D. P. et al. Immunohistochemical detection of PD-L1 among diverse human neoplasms in a reference laboratory: observations based upon 62,896 cases. Mod Pathol 32, 929-942 (2019).
Orczyk-Pawilowicz, M., Augustyniak, D., Hirnle, L. & Katnik-Prastowska, I. Lectin-based analysis of fucose and sialic acid expressions on human amniotic IgA during normal pregnancy. Glycoconj J 30, 599-608 (2013).
Osuga, T. et al. Relationship Between Increased Fucosylation and Metastatic Potential in Colorectal Cancer. J Natl Cancer Inst 108 (2016).
Pandey, A. et al. Glycosylation of Specific Notch EGF Repeats by O-Fut1 and Fringe Regulates Notch Signaling in Drosophila. Cell Rep 29, 2054-2066 e2056 (2019).
Pirozyan, M. R. et al. Pretreatment Innate Cell Populations and CD4 T Cells in Blood Are Associated With Response to Immune Checkpoint Blockade in Melanoma Patients. Front Immunol 11, 372 (2020).
Raulet, D. H. et al. Specificity, tolerance and developmental regulation of natural killer cells defined by expression of class I-specific Ly49 receptors. Immunol Rev 155, 41-52 (1997).
Rillahan, C. D. et al. Global metabolic inhibitors of sialyl- and fucosyltransferases remodel the glycome. Nat Chem Biol 8, 661-668 (2012).
Rock, K. L., Reits, E. & Neefjes, J. Present Yourself! By MHC Class I and MHC Class II Molecules. Trends Immunol 37, 724-737 (2016).
Rodig, S. J. et al. MHC proteins confer differential sensitivity to CTLA-4 and PD-1 blockade in untreated metastatic melanoma. Sci Transl Med 10 (2018).
Rossjohn, J. et al. T cell antigen receptor recognition of antigen-presenting molecules. Annu Rev Immunol 33, 169-200 (2015).
Rubio, G. et al. Cross-linking of MHC class I molecules on human NK cells inhibits NK cell function, segregates MHC I from the NK cell synapse, and induces intracellular phosphotyrosines. J Leukoc Biol 76, 116-124 (2004).
Schneider, M., Al-Shareffi, E. & Haltiwanger, R.S. Biological functions of fucose in mammals. Glycobiology 27, 601-618 (2017).
Smith, P. L. et al. Conditional control of selectin ligand expression and global fucosylation events in mice with a targeted mutation at the FX locus. J Cell Biol 158, 801-815 (2002).
Spitzer, M. H. et al. Systemic Immunity Is Required for Effective Cancer Immunotherapy. Cell 168, 487-502 e415 (2017).
Steentoft, C. et al. Precision mapping of the human O-GalNAc glycoproteome through SimpleCell technology. EMBO J 32, 1478-1488 (2013).
Stem, L. J. et al. Crystal structure of the human class II MHC protein HLA-DR1 complexed with an influenza virus peptide. Nature 368, 215-221 (1994).
Tay, R. E., Richardson, E. K. & Toh, H. C. Revisiting the role of CD4(+) T cells in cancer immunotherapy-new insights into old paradigms. Cancer Gene Ther 28, 5-17 (2021).
Tay, R. E., Richardson, E. K. & Toh, H. C. Revisiting the role of CD4(+) T cells in cancer immunotherapy-new insights into old paradigms. Cancer Gene Ther (2020).
Tsiakas, K. et al. Mutation of the glycosylated asparagine residue 286 in human CLN2 protein results in loss of enzymatic activity. Glycobiology 14, 1C-5C (2004).
Walser-Kuntz, D. R., Weyand, C. M., Fulbright, J. W., Moore, S.B. & Goronzy, J.J. HLA-DRB1 molecules and antigenic experience shape the repertoire of CD4 T cells. Hum Immunol 44, 203-209 (1995).
Wang, H. et al. D-mannose ameliorates autoimmune phenotypes in mouse models of lupus. BMC Immunol 22, 1 (2021).
Wang, Y. et al. Loss of alpha1,6-fucosyltransferase suppressed liver regeneration: implication of core fucose in the regulation of growth factor receptor-mediated cellular signaling. Sci Rep 5, 8264 (2015).
Weber, J. S. et al. Nivolumab versus chemotherapy in patients with advanced melanoma who progressed after anti-CTLA-4 treatment (CheckMate 037): a randomised, controlled, open-label, phase 3 trial. Lancet Oncol 16, 375-384 (2015).
Wei, W. et al. Molecular mechanisms of missense mutations that generate ectopic N-glycosylation sites in coagulation factor VIII. Biochem J 475, 873-886 (2018).
Yang, S. et al. Melanoma cells transfected to express CD83 induce antitumor immunity that can be increased by also engaging CD137. Proc Natl Acad Sci USA 101, 4990-4995 (2004).
Yuhki, N. et al. Comparative genome organization of human, murine, and feline MHC class II region. Genome Res 13, 1169-1179 (2003).
Zhang, D. et al. D-mannose induces regulatory T cells and suppresses immunopathology. Nat Med 23, 1036-1045 (2017).
Zhi, L. et al. FTY720 blocks egress of T cells in part by abrogation of their adhesion on the lymph node sinus. J Immunol 187, 2244-2251 (2011).
Zou, X. et al. A standardized method for lectin microarray-based tissue glycome mapping. Sci Rep 7, 43560 (2017).
Zuazo, M. et al. Functional systemic CD4 immunity is required for clinical responses to PD-L1/PD-1 blockade therapy. EMBO Mol Med 11, e10293 (2019).

1. A method of increasing the number of monocyte-derived dendritic cells (moDCs) in a tumor microenvironment or site of infection of an infectious disease in a subject comprising administering to the subject an agent that increases the amount of fucosylation on myeloid cells.

2. The method of claim 1, wherein the agent that increases fucosylation comprises L-fucose, D-fucose, fucose-1-phosphate, or GDP-L-fucose.

3. The method of claim 1, wherein the agent that modulates fucosylation is administered orally.

4. The method of claim 1, further comprising administering to the subject an immune checkpoint inhibitor.

5. The method of claim 4, wherein the immune checkpoint inhibitor is selected from the group consisting of a PD-1 inhibitor, a PD-L1 inhibitor, and a CTLA 4 inhibitor;
wherein the PD-1 inhibitor is selected from the group consisting of lambrolizumab, OPDIVO® (Nivolumab), KEYTRUDA® (pembrolizumab), and pidilizumab;
wherein the PD-L1 inhibitor is selected from the group consisting of BMS-936559, TECENTRIQ® (Atezolizumab), IMFINZI® (Durvalumab), and BAVENCIO® (Avelumab); and
wherein the CTLA-4 inhibitor is YERVOY (ipilimumab).

6. The method of claim 4, wherein the agent that increases the amount of fucosylation is administered before or during administration of the immune checkpoint inhibitor.

7. The method of claim 1, further comprising administering to the subject an adoptive cell therapy.

8. The method of claim 7, wherein the adoptive cell therapy comprises the transfer of tumor infiltrating lymphocytes (TILs), tumor infiltrating NK cells (TINKs), marrow infiltrating lymphocytes (MILs), chimeric antigen receptor (CAR) T cells, and/of CAR NK cells, or a combination thereof.

9. The method of claim 1, wherein the infectious disease comprises an infection from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, MERS-CoV, Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

10. The method of claim 1, wherein the infectious disease comprises an infection from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, other Yersinia species, and Mycoplasma species.

11. The method of claim 1, wherein the infectious disease comprises an infection from a fungus selected from the group consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

12. The method of claim 1, wherein the infectious disease comprises a parasitic infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

13. The method of claim 1, wherein the cancer is a melanoma or breast cancer.

14. The method of claim 1, further comprising detecting whether the cancer is highly immunosuppressive prior to administration of the agent that increases the amount of fucosylation.

15. A method of increasing antigen presentation by moDCs in a tumor microenvironment, site of infection of an infectious disease, local site of an antigen from a vaccine, or the draining lymph node of a vaccine of a subject with a cancer or infection, said method comprising administering to the subject an agent that increases the amount of fucosylation on moDCs; wherein the increase in fucosylation causes an increase in the number and length of dendrites on moDCs thereby increasing antigen presentation.

16. The method of claim 15, wherein the antigen is a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, MERS-CoV, Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

17. The method of claim 15, wherein the antigen is a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, other Yersinia species, and Mycoplasma species.

18. The method of claim 15, wherein the antigen is a fungal antigen from a fungus selected from the group consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

19. The method of claim 15, wherein the antigen is from a parasitic infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

20. The method of claim 15, wherein the antigen is a cancer antigen from melanoma or breast cancer.

21. The method of claim 15, wherein the antigen is produced by a vaccine.

22. The method of claim 21, wherein the vaccine comprises an mRNA, peptide, protein, heat killed infectious agent, or live attenuated infectious agent.

23. The method of claim 21, wherein the agent that increases the amount of fucosylation and the vaccine are administered prior to any infection with an infectious agent.


SEQUENCE LISTING (24 sequences)

SEQ ID NO: 1 - DNA, 45 bp, synthetic construct
  source 1..45: mol_type = other DNA
cgcgcgcgggatccatggagcagtcagagggagtcaattggactg

SEQ ID NO: 2 - DNA, 37 bp, synthetic construct
  source 1..37: mol_type = other DNA
cgcgcgcggctagcggtggtgcccacttcagagggcc

SEQ ID NO: 3 - DNA, 38 bp, synthetic construct
  source 1..38: mol_type = other DNA
gtctttgaaggatacacagccaccttaggatggactcg

SEQ ID NO: 4 - DNA, 32 bp, synthetic construct
  source 1..32: mol_type = other DNA
tgagtgtcatttcttcggtgggacggagcggg

SEQ ID NO: 5 - DNA, 38 bp, synthetic construct
  source 1..38: mol_type = other DNA
cgagtccatcctaaggtggctgtgtatccttcaaagac

SEQ ID NO: 6 - DNA, 38 bp, synthetic construct; identical to SEQ ID NO: 3
  source 1..38: mol_type = other DNA

SEQ ID NO: 7 - DNA, 29 bp, synthetic construct
  source 1..29: mol_type = other DNA
cgcgcccgggcgcgccatggtggtgtggc

SEQ ID NO: 8 - DNA, 26 bp, synthetic construct
  source 1..26: mol_type = other DNA
cgcgcccgctcgaggctcaggagtcc

SEQ ID NO: 9 - DNA, 28 bp, synthetic construct
  source 1..28: mol_type = other DNA
gtgtcatttctacggcgggacgcagcgc

SEQ ID NO: 10 - DNA, 28 bp, synthetic construct
  source 1..28: mol_type = other DNA
gcgctgcgtcccgccgtagaaatgacac

SEQ ID NO: 11 - DNA, 18 bp, synthetic construct
  source 1..18: mol_type = other DNA
aagcagactgccgcaaat

SEQ ID NO: 12 - DNA, 21 bp, synthetic construct
  source 1..21: mol_type = other DNA
ggcctgtaacgatgaggtttc

SEQ ID NO: 13 - DNA, 20 bp, synthetic construct
  source 1..20: mol_type = other DNA
acttccgccgagatctgttc

SEQ ID NO: 14 - DNA, 20 bp, synthetic construct
  source 1..20: mol_type = other DNA
ggatcagtggacgtaggcag

SEQ ID NO: 15 - DNA, 20 bp, synthetic construct
  source 1..20: mol_type = other DNA
gaacacgcttcttccttggg

SEQ ID NO: 16 - DNA, 21 bp, synthetic construct
  source 1..21: mol_type = other DNA
caggctccttacctttctggt

SEQ ID NO: 17 - DNA, 16 bp, synthetic construct
  source 1..16: mol_type = other DNA
ccgcggacgctggata

SEQ ID NO: 18 - DNA, 18 bp, synthetic construct
  source 1..18: mol_type = other DNA
ggcgattcgcgacttctg

SEQ ID NO: 19 - DNA, 20 bp, synthetic construct
  source 1..20: mol_type = other DNA
ccatagtagctcagcacccg

SEQ ID NO: 20 - DNA, 20 bp, synthetic construct
  source 1..20: mol_type = other DNA
gtcctgtcctgttctccagc

SEQ ID NO: 21 - DNA, 15 bp, Homo sapiens
  source 1..15: mol_type = genomic DNA
gagaacaaaatgttt

SEQ ID NO: 22 - DNA, 15 bp, Homo sapiens
  source 1..15: mol_type = genomic DNA
aagaacagcctgggc

SEQ ID NO: 23 - DNA, 15 bp, Homo sapiens
  source 1..15: mol_type = genomic DNA
gagcacagtgagtgc

SEQ ID NO: 24 - DNA, 15 bp, Homo sapiens
  source 1..15: mol_type = genomic DNA
aaggacagagtgaat